(19) United States
(12) Patent Application Publication
(10) Pub. No.: US 2025/0312436 A1
(43) Pub. Date: Oct. 9, 2025

(54) STABILIZATION OF VIRUS-BASED THERAPEUTIC AGENT

(71) Applicant: ELAREX INC., Burlington (CA)

(72) Inventor: Jeremy Andrew IWASHKIW, Burlington (CA)

(21) Appl. No.: 19/170,865

(22) Filed: Apr. 4, 2025

Related U.S. Application Data
(60) Provisional application No. 63/575,126, filed on Apr. 5, 2024.

(51) Int. Cl. A61K 39/205; A61K 39/00; A61K 39/235; A61K 47/26; A61K 47/36; A61K 47/42

(52) U.S. Cl. CPC A61K 39/205; A61K 39/235; A61K 47/26; A61K 47/36; A61K 47/42; A61K 2039/5254

(57) ABSTRACT

A virus-based active agent is mixed with trehalose and water and dried. The mixture may also contain one or more of carbohydrate, such as pullulan and albumin. The mixture may be dried to a moisture content of 0.1-10%. The drying may be under vacuum but optionally without producing a foam. Some or all of the drying may be at a temperature in the range of 15-400C, or at a temperature in the range of 1-15° C., or both. The active agent may be based on a vesicular stomatitis virus (VSV) or an adenovirus (AdV). The dried mixture may be stored at a temperature in the range of 1-55° C. A composition includes a virus, which may be a derived or modified form of a virus such as VSV or AdV. The composition may be used for a virus-based vaccine.

### RELATED APPLICATION

This application claims the benefit of, and priority from, U.S. Provisional Application No. 63/575,126, filed on Apr. 5, 2024, which is incorporated by reference.

### FIELD

This specification relates to a method for stabilizing a virus-based active agent, for example a viral vector vaccine, and to a composition comprising a virus-based active agent.

### BACKGROUND

Vesicular stomatitis virus (VSV) is a negative stranded enveloped RNA virus of the Rhabdoviridae family. VSV has two known serotypes, New Jersey (VSNJV) and Indiana (VSIV), with various strains in each serotype. Recombinant VSV (rVSV) platforms have been proposed as vaccines for viral diseases in humans and have been studied, for example, for use as therapeutic cancer vaccines. rVSV platforms may have one or more genetic modifications, for example modifications to attenuate the virus or the addition of one or more antigenic inserts. rVSV platforms have mild pathogenicity in humans but can induce humoral and cellular immune responses. In one example, rVSV-EBOV, in which the glycoprotein of VSV is exchanged with Ebola glycoprotein, is effective to inhibit Ebola infection in humans at doses between 10⁶-10⁸ PFU/dose when administered by intra-muscular injection. Unfortunately, VSV is less thermally stable than many other viruses. The rVSV-EBOV vaccine is stored in a frozen liquid formulation at −70° C. and loses effectiveness rapidly when thawed.

Adenoviruses (AdV) are non-enveloped DNA viruses with many serotypes. AdV-vectored vaccines may be derived from chimpanzee serotypes or human serotypes, for example AdV serotype 5. Recombinant AdV vectors are typically more thermally stable than other viral vectors and accordingly may be useful for vaccines having longer, or higher temperature, storage requirements.

US Patent Application Publication No. US 2019/0111006 A1 describes a method of preserving one or more biological species in a polymer matrix comprising pullulan and trehalose. The method includes combining the one or more biological species, an aqueous pullulan solution and an aqueous trehalose solution and drying the resultant mixture to provide a solid polymeric matrix. In some examples, the biological species is a live-attenuated viral vaccine and an inactivated viral vaccine.

Toniolo et al., Spray dried VSV-vectored vaccine is thermally stable and immunologically active in vivo, Scientific Reports 10, Article number: 13349 (2020), describes stabilizing a VSV-based vaccine in compositions comprising one or more of trehalose, dextran and mannitol. The compositions were spray dried. A composition comprising trehalose and another composition comprising trehalose and dextran mixed at a 3:1 ratio produced about a 4 log PFU loss after 7 days of storage of the spray dried product at 37° C.

Berg et al., Stability of Chimpanzee Adenovirus Vectored Vaccines (ChAd0×1 and ChAd0×2) in Liquid and Lyophilised Formulations, Vaccines, 2021: 9(11):1249, describes stabilizing an AdV-based vaccine in compositions including, among other things, inulin and mannitol. A freeze-dried example had an infectivity loss of 2 log after storage at 45° C. and an infectivity loss of about 1.5 log after 60 days of storage at 30° C.

### SUMMARY

This specification describes a method of preserving and/or stabilizing a virus. The virus may be an active agent of a virus-based vaccine such as a viral vector vaccine. In some examples, the virus is a recombinant virus and/or a derived from a VSV or an AdV. The virus is mixed with trehalose, a buffer and water. Optionally, the mixture may also contain carbohydrate (for example pullulan), albumin or both. In some embodiments, the mixture is dried under a pressure and a temperature such that the virus is present in solution above 10° C. for 90 minutes or less and/or such that the mixture is a liquid solution for 120 minutes or less. In some embodiments, the mixture is maintained above freezing and/or does not foam. In some embodiments, the mixture is dried by foam drying and/or has a temporary excursion below a freezing temperature. The mixture may be dried to a moisture content of 1 to 10%. In some examples, the drying is at two or more temperatures. The dried mixture may be stored, for example at a temperature in the range of 1-55° C. Optionally, the dried mixture may be dissolved in water, optionally in the form of an aqueous buffer, to form an injectable liquid vaccine.

This specification also describes a composition. The composition includes a virus. The virus may be an active agent of a virus-based vaccine such as a live-attenuated viral vaccine or a viral vector vaccine. In some examples, the virus is a recombinant virus and/or a derived from a VSV or an AdV. The composition also includes trehalose and a buffer. The composition may also include carbohydrate (for example pullulan), albumin or both. The composition may have a moisture content of 1 to 10%. The composition may include a glass, optionally a foamed glass, incapsulating the virus. The composition may be used, for example, in a virus-based vaccine such as a viral vector vaccine.

Experimental examples are provided using adenovirus and VSV based constructs. Adenovirus is inherently more thermally stable than VSV. Some aspects of the invention are applicable to both adenovirus and VSV and are therefore expected to be applicable to the thermal stability of viral-vectored vaccines or other therapeutic agents generally. Other aspects of the invention may be applicable in particular to adenovirus or VSV based agents.

VSV is believed to degrade rapidly under some conditions, for example when in solution above about 10° C. In experimental examples described herein, VSV is stored on ice while being formulated. During the formulation step, the samples are at about 25° C. for about 30 minutes before being placed on a shelf in a freeze dryer (which is not necessarily used to provide freeze drying).

In a foam VSV drying method, the freeze dryer may be set to a shelf temperature below 10° C., for example 4° C., and allowed to equilibrate for about 30 minutes. The vacuum is then turned on to a low pressure setting, for example 20 ubar or less. Without a change in the shelf temperature, the sample temperature (i.e. the temperature measured by a probe in the sample vial) drops within minutes, for example within 5 minutes, of turning on the vacuum. The sample temperature may drop to below freezing, for example −14° C., for a period of time. Vapor bubbles are produced in the sample and a continuous solid phase is not produced despite the sub-freezing temperature, although it is possible that discontinuous solid volumes may exist. After a short period of time, for example about 8 minutes, with the vacuum on the sample temperature begins to rise indicating that the rate of evaporation is decreasing. After a further period of time, for example around 60 minutes, the sample temperature reaches the shelf temperature indicating that the rate of evaporation has declined significantly. It is hypothesized that at some point during these time periods, the moisture content of the sample may have been reduced to such an extent that the sample is no longer a solution, but rather a moist gel or solid (i.e. a moist glass). The set point temperature of the dryer may later be increased above 10° C. to reduce the residual moisture content of the sample without the rapid degradation of VSV observed in solution at such temperatures. The dried sample is optionally a foamed solid.

In a gentle VSV drying process, the freeze dryer may be set to a higher shelf temperature that is still below 10° C., for example 8° C. An equilibration period is typically not provided. The vacuum is turned on, but to a higher set point, for example 1-20 mbar. The sample temperature drops due to evaporative cooling, for example to around 5-6° C., within a short time, for example around 15 minutes. There is no excursion below the freezing temperature of the sample. Bubbling is not observed in the sample in at least some examples. The sample temperature later starts to increase towards the shelf temperature set point, for example after about 90 minutes, and quickly reaches the shelf temperature set point, for example after about another 5 minutes. It is hypothesized that at some point during these time periods, the moisture content of the sample may have been reduced to such an extent that the sample is no longer a solution, but rather a moist solid (i.e. a moist glass). The set point temperature of the dryer may then be increased above 100° C. to reduce the residual moisture content of the sample without the rapid degradation of VSV observed in solution at such temperatures. The dried sample is optionally a solid film (non-foamed).

In some discussion herein an intermediate composition is described existing before the temperature set point is increased. The words “intermediate composition” are not meant to necessarily require any chemical change, there may be only a phase, state or water content change from a liquid solution to, for example, a moist gel, glass or solid.

By way of either method described above, the VSV or other sample is present in solution above 100° C. for only a short period of time, for example 90 minutes or less. Most of this time is for sample preparation and could be reduced or avoided by working under cold conditions, optionally with robotic handing equipment. Further, the sample is no longer a liquid solution within 120 minutes or less of the start of the drying process, i.e. within 120 minutes or less of entering the freeze dryer. The drying process continues after this time to reduce the residual moisture content but a material physical change has already occurred and the sample is less susceptible to degradation at temperatures above 10° C. Without intending to be limited by theory, the short time period during which the sample is in solution above 10° C. and/or the short time period during which the sample is a liquid solution (at any temperature), may assist in reducing process loss and/pr producing a dried sample that is thermally stable over time. Adenovirus might not require limiting the time period during which the sample is in solution above 10° C. but may still benefit from a short time period during which the sample is a liquid solution (at any temperature).

At least with VSV (though not necessarily with adenovirus or other vectors), foam drying may produce slightly better long term stability than gentle drying. However, gentle drying can produce a film form product, which can be advantages for some forms of vaccine delivery such as a strip applied to a patient's cheek or under the tongue.

In some examples herein, trehalose is present at 3-10 times the amount of a carbohydrate such as pullulan, and/or at least 12.5% trehalose. For example, a formulation may have 2.5% pullulan and 15% trehalose. Formulations with at least 3 times as much trehalose as carbohydrate/pullulan and/or with at least 12.5% trehalose, may be particularly useful in combination with adenovirus.

In some examples, pullulan is replaced by another carbohydrate such as CMC or Dextran. In the presence of albumin and after a short period of storage (1 week, the only data available at the time of filing this application) alternate polymers produce no statistically significant reduction in performance relative to pullulan, at least while stabilizing VSV. However, it is possible that differences in stability may emerge after longer periods of storage. Additional short term data indicates that in the presence of albumin, removing pullulan produces no statistically significant reduction in performance relative to formulations with pullulan, at least while stabilizing VSV. However, differences in stability, wherein the addition of pullulan was beneficial, emerged after longer periods of storage in formulations without albumin and may emerge after longer periods of storage with albumin.

In other examples, the addition of a surfactant, such as PMAL-16, caused an increase in process loss, at least with adenovirus. Preferred formulations may be essentially without added surfactant. A formulation may consist essentially (i.e. 95% or more or 98% or more on a dry basis) of a polymer (for example pullulan), a saccharide (such as trehalose), a buffer (such as Tris), albumin, and the virus being stabilized.

Other features and advantages of the present application will become apparent from the following detailed description. It should be understood, however, that the detailed description and the specific examples, while indicating embodiments of the application, are given by way of illustration only and the scope of the claims should not be limited by these embodiments but should be given the broadest interpretation consistent with the description as a whole.

### DRAWINGS

Other features and the embodiments of the application will now be described in greater detail with reference to the attached drawings in which:

FIGS. 1A, 1B and 1C show the results of the analysis of an exemplary pullulan/trehalose (PT)/CM/BSA/NaCl composition and a comparative sorbitol and gelatin (control) composition, both dried at 20° C. including Residual Moisture (RM) (FIG. 1A), b) titer measured after storage at 37° C. for 189 days (FIG. 1B), and c) total log loss measured for 22 weeks (FIG. 1C).

FIGS. 2A to 2G show the results of the analysis of exemplary PT/CM/BSA/NaCl and Tris/Trehalose compositions dried at 4° C./25° C. including FIG. 2A: RM, FIG. 2B: total log loss measured for 32 weeks at 4° C., FIG. 2C: total log loss measured for 32 weeks at 25° C., FIG. 2D: total log loss measured for 32 weeks at 37° C., FIG. 2E: titer time course measured for 32 weeks at 4° C., FIG. 2F: titer time course measured for 32 weeks at 25° C., and FIG. 2G: titer time course measured for 32 weeks at 4° C.

FIGS. 3A-3E show the results of the analysis of exemplary PT/CM/BSA/NaCl and Tris/Trehalose compositions in FBS free media dried at 4° C./25° C. including FIG. 3A: total protein, FIG. 3B: RM, FIG. 3C: titer time course measured for 14 days at 37° C., FIG. 3D: total log loss measured for 14 days, and FIG. 3E: total log loss measured for 14 days at 37° C.

FIG. 4 shows the results of the RM analysis of exemplary PT/CM/BSA/NaCl composition in FBS free media with various amounts of BSA, pullulan and trehalose, dried at 4° C./25° C..

FIGS. 5A and 5B show the results of analysis of the exemplary PT/CM/BSA/NaCl compositions in FIG. 4 including FIG. 5A: titer time course measured for 14 days, and FIG. 5B: stability time course measured for 14 days at 37° C.

FIGS. 6A-6G show the results of the analysis of exemplary PT/CM/BSA/NaCl and Tris/Trehalose compositions dried at 4° C./25° C. including FIG. 6A: RM, FIG. 6B: titer time course measured for 20 weeks at 4° C., FIG. 6C: titer time course measured for 20 weeks at 25° C., FIG. 6D: titer time course measured for 4 weeks at 37° C., FIG. 6E: total log loss measured for 20 weeks at 4° C.,

FIG. 6F: total log loss measured for 20 weeks at 25° C., FIG. 6G: total log loss measured for 4 weeks at 37° C.,

FIG. 7 is a bar chart of results of the analysis of exemplary PT/CM/BSA/NaCl composition dried at 4° C./25° C. with varying P/T concentrations showing titer loss measured for 14 days.

FIG. 8 is a line graph corresponding to FIG. 7.

FIG. 9 shows the RM of exemplary PT/CM/BSA/NaCl compositions with varying treatment of viral stock and sucrose addition dried at 15° C./25° C.

FIG. 10 shows the total log loss measured for 14 days at 37° C. for the formulations in FIG. 9.

FIG. 11 shows the RM of various exemplary compositions.

FIG. 12 shows the titer of the compositions of FIG. 11 after storage at 37° C.

FIG. 13 shows the loss of titer of the compositions of FIG. 11 after storage at 37° C.

FIG. 14 shows the RM of various exemplary compositions.

FIG. 15 shows the titer of the compositions of FIG. 14 after storage at 37° C.

FIG. 16 shows the loss of titer of the compositions of FIG. 14 after storage at 37° C.

FIG. 17 shows infectivity unit (IU) loss of two AdV formulations after foam drying and storage at 37° C.

FIG. 18 shows infectivity unit (IU) loss of the two AdV formulations of FIG. 17 after foam or freeze drying and storage at 37° C.

FIG. 19 shows infectivity unit (IU) loss of three AdV formulations stored at 37° C.

FIG. 20 shows infectivity unit (IU) loss of an AdV formulation stored at three temperatures.

FIG. 21 shows infectivity unit (IU) loss of two AdV formulations stored at 55° C.

FIG. 22 shows the RM of exemplary compositions with 5% and 15% trehalose.

FIG. 23 shows gentle drying cycle of exemplary compositions with 5% and 15% trehalose.

FIG. 24 shows the loss of titer of compositions dried at low and high vacuum drying after storage at 37° C.

FIG. 25 shows the loss of titer of the compositions with 5% and 15% trehalose after storage at 4° C.

FIG. 26 shows the loss of titer of the compositions with 5% and 15% trehalose after storage at 25° C.

FIG. 27 shows the loss of titer of the compositions with 5% and 15% trehalose after storage at 37° C.

FIG. 28 shows the RM of exemplary compositions with pullulan or alternative polymers.

FIG. 29 shows the loss of titer of exemplary compositions with pullulan, CMC or dextran after storage at 4° C.

FIG. 30 shows the loss of titer of exemplary compositions with pullulan, CMC or dextran after storage at 25° C.

FIG. 31 shows the loss of titer of exemplary compositions with pullulan/trehalose, pullulan or trehalose after storage at 4° C.

FIG. 32 shows the loss of titer of exemplary compositions with pullulan/trehalose, pullulan or trehalose after storage at 25° C.

FIG. 33 shows the RM of exemplary compositions with CM PT BSA NaCl and Inulin/Mannitol after foam drying.

FIG. 34 shows the RM of exemplary compositions with CM PT BSA NaCl and Inulin/Mannitol after freeze drying.

FIG. 35 shows the loss of titer of exemplary compositions with CM PT BSA NaCl and Inulin/Mannitol after foam or freeze drying and after storage at 37° C.

FIG. 36 shows the loss of titer of exemplary compositions with CM PT BSA NaCl after foam drying and after storage at 4, 30, 37 and 45° C.

FIG. 37 shows the SP dryer probe temperatures for exemplary formulations of the application.

FIG. 38 shows the RM of exemplary compositions of the application.

FIG. 39 shows the loss of titer of exemplary compositions of the application after storage at 55° C.

FIG. 40 shows the RM of exemplary compositions of the application.

FIG. 41 shows the loss of titer of exemplary compositions of the application after storage at 55° C.

FIG. 42 shows the RM of exemplary compositions of the application.

FIG. 43 shows the loss of titer GFP of exemplary compositions of the application after storage at 55° C.

FIG. 44 shows the loss of titer Luc of exemplary compositions of the application after storage at 55° C.

FIG. 45 shows the RM of exemplary compositions of the application.

FIG. 46 shows the loss of titer of exemplary compositions of the application after storage at 55° C. (Adv Vectored Vaccine).

FIG. 47 shows the loss of titer Luc of exemplary compositions of the application after storage at 55° C. (Adv-GFP Stability).

FIG. 48 shows VSV stability with air drying methodology. A) VSV stability for process loss and thermal challenge at 37° C. over 14 days. B) Calculated means and standard deviation for each data point of accumulated Log PFU loss for each condition.

FIG. 49 shows stability of VSV dried by freeze dry methodology. A) Cake morphology after formulation and drying. Cake-like structures were observed for both F2 and F5, whereas collapsed cakes were observed for F3 and F4. B) VSV stability for process loss and thermal challenge at 37° C. over 7 days. C) Calculated means and standard deviation (SD) for each data point of accumulated Log PFU loss for each condition. Each data point is collected from duplicate serial dilution plating of biological duplicate vials.

FIG. 50 shows stability of VSV dried by Vacuum foam dry methodology. A) Cake morphology after formulation and drying. No film was observed for F6, whereas bubbly foam was observed for F7/F8. B) VSV stability for process loss and thermal challenge over 14 days. Calculated means and standard deviation (SD) for each data point of accumulated Log PFU loss for each condition. Each data point is collected from duplicate serial dilution plating of biological duplicate vials.

FIG. 51 shows the role of BSA in stabilizing VSV dried by Vacuum foam dry methodology. A) Protein quantification of VSV lots before and after dialysis into formulation buffer. B) VSV stability for process loss and thermal challenge over 14 days at 37° C.. C) Calculated means and standard deviation (SD) for each data point of accumulated Log PFU loss for each condition. Each data point is collected from duplicate serial dilution plating of biological duplicate vials.

FIG. 52 shows long term stability of VSV dried by Vacuum foam dry methodology. A) Film morphology. B) VSV stability for process loss and thermal stability over 27 weeks at 37° C. Solid line represents one phase decay regression model with dotted lines for 95% confidence interval. C) Calculated means and standard deviation (SD) for each data point of accumulated Log PFU loss for each condition. Each data point is collected from duplicate serial dilution plating of biological duplicate vials.

FIG. 53 shows comparison of different dry schedules for VSV process and thermal stability loss over 14 days. A) VSV stability for process loss and thermal challenge over 14 days. B) Calculated means and standard deviation for each data point of accumulated Log PFU loss for each condition.

FIG. 54 shows long term stability of VSV formulated with F2 or F12 and dried with 4/25° C. dry schedule at 3 different thermal challenge temperatures. A) Dried film morphology. B) VSV stability at 4° C. thermal challenge. C) VSV stability at 25° C. thermal challenge. D) VSV stability at 37° C. thermal challenge. For panels B-D, Solid line represents one phase decay regression model with dotted lines for 95% confidence interval. E) Calculated means and standard deviation (SD) for each data point of accumulated Log PFU loss for each condition. Each data point is collected from duplicate serial dilution plating of biological duplicate vials.

FIG. 55 shows XRD traces of formulations over 14-day time course at 37° C.. A) Formulation F2 in vials backfilled with dried nitrogen. B) Formulation F2 in vials backfilled with atmosphere. C) Formulation F2 with inactivated VSV backfilled with dried nitrogen. D) Formulation F13 in vials backfilled with dried nitrogen. B) Formulation F13 in vials backfilled with atmosphere. C) Formulation F13 with inactivated VSV backfilled with dried nitrogen.

FIG. 56 shows XRD analysis of dried films over 14 days incubated at 37° C.. Percent crystallinity was calculated by determining the area under the curve (AuC) of amorphous and crystalline material and dividing values.

FIG. 57 shows stability time course at 3 different incubation temperatures of VSV dried with 4/25° C. dry schedule and backfilled with dried N₂ gas. A) Dried film morphology. B) VSV stability at 4° C. thermal challenge. C) VSV stability at 25° C. thermal challenge. D) VSV stability at 37° C. thermal challenge. For panels B-D, Solid line represents one phase decay regression model with dotted lines for 95% confidence interval. E) Calculated means and standard deviation (SD) for each data point of accumulated Log PFU loss for each condition. Each data point is collected from duplicate serial dilution plating of biological duplicate vials.

FIG. 58 shows dryer probe temperatures of standard drying method for exemplary formulations of the application.

### DETAILED DESCRIPTION

### 1. Definitions

Unless otherwise indicated, the definitions described in this and other sections are intended to be applicable to all embodiments and aspects of the present application herein described for which they are suitable as would be understood by a person skilled in the art.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least +5% of the modified term if this deviation would not negate the meaning of the word it modifies.

As used in this application, the singular forms “a”, “an” and “the” include plural references unless the content clearly dictates otherwise. For example, an embodiment including “a biomolecule” should be understood to present certain aspects with one biomolecule or two or more additional biomolecules.

In embodiments comprising an “additional” or “second” component, such as an additional or second biomolecule, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The term “process loss” as used herein refers to a loss in titer during the process of composition formulation (i.e. buffer exchange and addition of further excipient solution) and drying. Without intended to be limited by theory, process loss appears to be primarily related to the drying procedure. The term “storage stability” as used herein refers to titer loss, if any, during storage of the dried product at one or more temperatures. The term “stability” as used herein may refer to process loss or storage stability or both. Concentrations given in % herein are w/v unless stated otherwise.

The term “method of the application” or “method of the present application” and the like as used herein refers to a method of preserving and/or stabilizing a virus.

The term “composition of the application” or “composition of the present application” and the like as used herein refers to a composition comprising a virus.

The term “preserving” or “preservation” as used herein with respect to the virus means to maintain at least a measurable or detectable level of function or activity for the virus for a desired period of time under specified conditions.

The term “stabilizing” or “stabilization” as used herein with respect to a virus refers to any reduction in the degradation or loss of activity of the virus compared to a control.

The term “pullulan” as used herein refers to a polysaccharide polymer comprising maltotriose units. Optionally, pullulan may be a natural polysaccharide which is produced by Aurebasidium pullulans. The pullulan used in examples herein has a molecular mass of 200 kDa.

The term “trehalose” as used herein refers to a disaccharide commonly used as a cryoprotectant. Trehalose may be (D)-(+)-trehalose which is a disaccharide composed of two glucose molecules bound together via the α,α-1,1-glucosidic linkage.

The term “vaccine” as used herein may mean, where appropriate given the context, an antigen of a vaccine, but does not necessarily exclude the presence of other parts of a vaccine, such as an adjuvant or diluent.

The term “essentially free from” as used herein means that the presence of the stated features, elements, or components, is in an amount that does not materially affect the characteristics of the composition or material being referenced.

As used herein, the term “effective amount” or “therapeutically effective amount” means an amount that is effective, at dosages and for periods of time necessary, to achieve a desired result.

As used herein, the term “high molecular weight surfactants” means a surface active, amphiphilic molecule greater than 1500 molecular weight.

### II. Methods of the Application

The present application includes a method of preparing a dry preserving and/or stabilizing composition comprising a virus, the method includes combining the virus with trehalose, water and a buffer, optionally with carbohydrate (for example pullulan) and/or albumin, to produce a composition; and drying the composition to produce a dried composition, wherein drying the composition comprises removing solvent from the composition under a pressure and a temperature sufficient to provide a short time period during which the sample is in solution above 100° C. and/or a short time period during which the sample is a liquid solution (at any temperature), thereby producing the dry preserving and/or stabilizing composition comprising the virus.

In some embodiments, the solvent is removed by evaporation. In some embodiments, the solvent is removed by boiling. In some embodiments, the solvent is removed by simultaneous evaporation and boiling. In some embodiments, boiling (foaming) generates bubbles and the solvent is removed during the bubbling of the composition to form a moist solid and then when the bubbling is stopped moisture is removed from the moist solid to produce the dried composition.

In some embodiments, the dried composition may include a glass encapsulating particles of the virus. In some embodiments the glass is a foamed glass. In some embodiments the glass is a non-foamed solid.

In some embodiments, the virus is a virus in any recombinant, derived or modified form. In some embodiments, the virus is a live-attenuated virus, an inactivated virus, a viral vector or a recombinant virus. In some embodiments, the virus is an RNA virus, optionally an enveloped RNA virus. In some embodiments, the virus is a vesicular stomatitis virus (VSV). In some embodiments, the VSV is a recombinant VSV. In some embodiments, the virus is an adenovirus or derived from an adenovirus. In some embodiments, the virus is formulated for administration in a biological preparation. In some embodiments, the virus is formulated for administration as a vaccine.

In some embodiments, samples of virus may be provided from suppliers in a buffer and may contain remnants of the virus manufacturing process. In some embodiments, the samples of virus may be purified to remove manufacturing process remnants according to any purification method known in the art. In some embodiments, a buffer exchange may be performed to substantially replace a buffer originally supplied with the virus samples with a new buffer. In some embodiments, when a virus sample or originally supplied sample buffer is used that contains any component of a composition described in the present application, the amount of the component may be adjusted to account for the amount carried over from the virus sample or the originally supplied buffer.

In some embodiments, the buffer is any buffer that maintains the pH of the composition of the application within the range of 6.8 to 8.2. In some embodiments, the buffer is a CM buffer or a tris(hydroxymethyl)aminomethane (Tris) buffer. In some embodiments, the buffer is a CM buffer. The CM buffer is prepared by mixing 2.5 g MgSO₄*7H₂0 (10 mM), 0.735 g CaCl₂) (10 mM), 0.05 g gelatin (0.005 mM) and 6 mL 1 M Tris-HCl (50 mM), with water for a final volume of 1 L. In some embodiments, the buffer is a Tris buffer or a Tris-HCl buffer. The Tris buffer includes 10-50 mM of Tris. In some embodiments, the buffer maintains the pH of the composition in the range of 6.8 to 8.2, in the range of 6.9 to 8,1, or in the range of 7.2-7.5. No differences in stability have been detected for compositions having pH in a range of 6.9 to 8.1. Tris and Tris-based buffers are suitable for compositions having a pH of at least 7. In some embodiments, the buffer is present in a liquid composition at a concentration of about 5 mM to about 20 mM. In some embodiments, the buffer is present in the dry composition at a concentration of about 0.5 wt % to about 10 wt %. In some embodiments, the buffer is present in the dry composition at a concentration of about 1 wt %, about 1.5 wt %, about 2 wt %, about 4 wt %, or about 8 wt % and values therebetween.

In some embodiments, other buffers, for example a Histidine buffer, may be used. In some embodiments, the buffer does not contain substantial amounts of crystal forming components. For example, phosphate-buffered saline (PBS) might reduce the performance of the composition of the application. In some embodiments, CM buffer may produce sulfate crystals and accordingly a Tris-HCl buffer or other buffer may be preferred over a CM buffer in some examples.

In some embodiments, the trehalose is present in the liquid composition at a concentration of about 1.25% (w/v) to about 20% (w/v). In some embodiments, the trehalose is present in the composition at a concentration of about 1.25% (w/v), about 2.5% (w/v), about 5% (w/v), about 10% (w/v), or about 15% (w/v). Trehalose is available from a variety of commercial sources. In some embodiments, the trehalose is present in the dry composition in a concentration of about 30 wt % to about 70 wt %, or about 70 wt % to 99 wt %. In some embodiments, the trehalose is present in the dry composition in a concentration of about 35% wt %, about 35 wt %, about 40 wt %, about 45 wt %, about 50 wt %, about 55 wt %, about 60 wt %, about 65 wt %, or about 80 wt %, about 90 wt %, or about 95 wt % and values therebetween.

In some embodiments, the carbohydrate is selected from carboxymethylcellulose (CMC), dextran or pullulan. In some embodiments the carbohydrate is pullulan.

In some embodiments, the ratio of trehalose to carbohydrate is in the range of 4:1 to 0.5:1 by weight, for example about 2:1. In some embodiments, the ratio of trehalose to carbohydrate is about 1:1. In some embodiments, the ratio of trehalose to carbohydrate is about 3:1.

In some embodiments, the carbohydrate is present in the composition in a concentration of about 0.5% to about 15%. In some embodiments, the carbohydrate is present in the composition at a concentration of about 0.625%, about 1.25%, about 2.5%, about 5%, or about 10%. In some embodiments, the carbohydrate is present in the dry composition in a concentration of about 15% wt % to about 50 wt %. In some embodiments, the carbohydrate is present in the dry composition in a concentration of about 20 wt %, about 25 wt %, about 25 wt %, about 30 wt %, about 35 wt %, about 40 wt %, or about 45 wt % and values therebetween.

In some embodiments, the carbohydrate is pullulan. In some embodiments the pullulan has a molecular weight in the range of about 100,000 to about 200,000. Pullulan having such molecular weights is commercially available.

In some embodiments, unless stated otherwise and when the composition comprises trehalose and pullulan, “PT” refers to a composition having pullulan and trehalose. Optionally, different ratios of pullulan to trehalose may be used. PT solutions may be viscous and can be prepared independently of a virus containing buffer and then added to the virus containing buffer, for example at a ratio of PT solution to virus containing buffer in the range of 1:1 to 9:1 or 4:1 to 9:1 volume. In some embodiments, the ratio of trehalose to pullulan is in the range of 4:1 to 0.5:1 by weight, for example about 2:1. In some embodiments, the ratio of trehalose to pullulan is about 1:1. In some embodiments, the ratio of trehalose to pullulan is about 3:1. The PT solution is optionally made by dissolving the pullulan and trehalose into an additional volume of the same buffer used in the virus containing buffer.

In some embodiments, the virus is grown in a fetal bovine serum free media. In some embodiments, when the virus stock has protein, buffer exchange removes substantial amounts of the protein. As such, in some embodiments, the dialysed (i.e. buffer exchanged) solutions of the virus have minimal protein, typically less than 50 μg/mL and are considered albumin free, unless albumin is added to the composition of the application.

In some embodiments, the composition comprises albumin. In some embodiments, the albumin can be added, for example as human serum albumin (HAS), bovine serum albumin (BSA) or a recombinant albumin, for example recombinant human albumin. The pH of a composition may need to be adjusted to avoid a decrease in pH after adding albumin. In some embodiments, the concentration of the albumin in the composition is in the range of 0.125%-2.5%. In some embodiments, the concentration of the albumin in the composition is about 0.5% or about 2%. In some embodiments, albumin improves stability of the virus in the composition and/or reduces process loss in the method of preparing the dry composition. Lower concentrations of albumin, for example in the order of 0.05%, may be provided in some samples by way of carryover from the virus manufacturing process. While low concentrations of albumin from the carryover protein can improve stability and reduce process loss, these lower concentrations are not as effective as higher concentrations. In a commercial vaccine manufacturing process there should be no protein carryover and albumin can be added to the liquid compositions, for example in the range of 0.125%-2.5% described above, without adjusting for albumin carryover. BSA is used in examples described herein for convenience. However, in a human vaccine HSA or a recombinant human albumin may be used. In an animal vaccine, a form of albumin acceptable to the animal may be used. In some embodiments, the ratio of trehalose to albumin is from 3:1 to 25:1. In some embodiments, the ratio of trehalose to albumin is 10:1. In some embodiments, the albumin is present in the dry composition in a concentration of about 2 wt % to about 20 wt %. In some embodiments, the albumin is present in the dry composition in a concentration of about 2.5 wt %, about 4 wt %, about 7 wt %, or about 15 wt % and values therebetween.

In some embodiments, the composition comprises NaCl. NaCl may be present in the composition at a concentration of 25-150 mM or 50-100 mM. NaCl may be present in the composition at a concentration of 50 mM. Compositions with higher concentrations of NaCl (for example 250 mM) may have increased residual moisture, process loss or reduced storage stability. In some embodiments, NaCl was found to be beneficial in combination with a CM buffer but might not be beneficial in combination with a Tris-HCl buffer. In some embodiments, the NaCl is present in the dry composition in a concentration of about 1 wt % to about 12 wt %. In some embodiments, the NaCl is present in the dry composition in a concentration of about 1.5 wt %, about 2 wt %, about 3 wt %, about 4 wt %, about 6 wt %, or about 10 wt % and values therebetween.

An exemplary composition and an optional range of parts by mass (excluding water) for other compositions are described in some of the tables below. In some embodiments, the composition comprises about 23 to about 300 parts of the trehalose. In some embodiments, the composition comprises about 1 to about 10 parts of the buffer. In some embodiments, the composition comprises about 12 to about 150 parts carbohydrate. In some embodiments, the composition comprises about 2 to about 35 parts of the albumin. In some embodiments, the composition comprises about 2 to about 12 parts of the NaCl. In some embodiments, the composition comprises about 0.0001 to about 0.01 parts of the virus.

In some embodiments, in the method of the application the drying of the composition comprises a step of foam drying (boiling). In some embodiments, foam drying includes a process that is carried out under vacuum sufficient to cause the composition to foam. In some embodiments, the foam drying is vacuum foam drying or vacuum foam freeze drying.

In some embodiments, the method of the application includes vacuum drying but is free from foam drying (boiling).

In some embodiments, the method of the application includes maintaining the temperature of the composition during the drying step below about 10° C. and above a freezing point. The composition (or the slurry) containing the virus is optionally kept below 10° C. for all but a short period of time during the step of removing the solvent from the composition of the application for substantial part of the process.

In some embodiments, the composition is maintained above about 100° C. in solution during the method of the present application. In some embodiments, the composition is maintained above about 100° C. in solution during the step of producing the composition (the step of formulation). In some embodiments, the method of the application comprises an additional step of preincubation and the composition is maintained above about 100° C. in solution during said preincubation. In some embodiments, during the step of drying, after the vacuum is turned on, the composition of the application is maintained above about 100° C. in solution for about 1 to about 20 min, or for about 15 min, or for about 5 min.

In some embodiments, during the method of the application, the composition is maintained above about 100° C. in solution for about 30 min to about 80 minutes, or for about 40 min to about 75 minutes or for about 45 min to about 70 minutes.

In some embodiments, the dry composition of the application essentially free from solvent is obtained after about 50 to about 100 minutes, or after about 60 to about 90 minutes, or after about 70 minutes, or about 80 minutes.

Drying temperature may have a material effect on at least one of residual moisture, process loss and storage stability. Drying at or near ambient temperature, for example about 20° C., produces low residual moisture, even in samples containing pullulan. As such, in some embodiments, at least part of the drying is carried out at a temperature of about 15° C. to about 40° C. In some embodiments, at least part of the drying is carried out at a temperature of about 1° C. to about 10° C. In some embodiments, the drying at a lower temperate at least initially, results in less process loss. In some embodiments, at least part of the drying is carried out at a temperature of about 20° C. In some embodiments, at least part of the drying is carried out at a temperature of about 4° C.

In some embodiments, the drying is carried out at two or more temperatures. In some embodiments, the drying is carried out at a temperature in the range of about −50° C. to about 15° C., or about 1° C. to about 100° C., optionally for 5-15 hours, followed directly or indirectly by drying at a higher temperature, for example about 15° C. to about 400° C., optionally for 5-15 hours.

The method of the application may result in low residual moisture (RM), for example in the range of 1-10% or 1-7%, even in compositions with pullulan. As such, the method of the application achieves RM of about 1% to about 10%. As such, in some embodiments, the dry composition has a water content of less than 10 wt %. In some embodiments, the dry composition has a water content of less than 9 wt %. In some embodiments, the dry composition has a water content of less than 8 wt %. In some embodiments, the dry composition has a water content of less than 7 wt %. In some embodiments, the dry composition has a water content of less than 6 wt %. In some embodiments, the dry composition has a water content of less than 5 wt %. In some embodiments, the dry composition has a water content of about 1 wt % to about 10 wt %. In some embodiment, the dry composition a water content of about 1 wt % to about 9 wt %. In some embodiment, the dry composition has a water content of about 1 wt % to about 8 wt %. In some embodiment, the dry composition has a water content of about 1 wt % to about 7 wt %.

It has been shown that further excipients such as polymers and amino acids such as PEG 200, PEG 4000, PEG 6000, Histidine, Glutamic acid, and polyvinylpyrrolidone such as polyvinylpyrrolidone K-15 (PVP K15) do not improve the stability of the composition of the application. As such, in some embodiments, the composition of the application is free from polymeric excipients. In some embodiments, the composition of the application is free from amino acids. The examples also showed that compositions with hydroxyectoine, ectoine, β-cyclodextran, ethylenediaminetetraacetic acid (EDTA) and sorbitol do not improve the stability.

The examples further showed that compositions with surfactants do not improve the stability of the composition of the application. In fact, it has been shown that composition with poly (Maleic Anhydride-Alt-1-Octadecene) substituted with 3-(Dimethylamino) Propylamine (PMAL-C16) lost titer after exchange of buffer into PMAL. As such, in some embodiments, the composition of the application is free from PMAL-C16.

In some embodiments, the composition of the application is free from high molecular weight surfactants such as Pluronic F127, Pluronic F68, Pluronic P123, or other EO-PO block copolymers of greater than 3,000-4,000 MW. In some embodiments, the composition of the application is free from any surfactants. Exemplary surfactants include, but are not limited to nonionic surfactants such as alkyl poly(ethylene oxide), copolymers of poly(ethylene oxide) and poly(propylene oxide) (EO-PO block copolymers), poly(vinyl pyrroloidone), alkyl polyglucosides (such as sucrose monostearate, lauryl diglucoside, or sorbitan monolaureate, octyl glucoside and decyl maltoside), hydroxypropyl methylcellulose (HPMC), poly(ethylene)glycol 3000, dodecyl-p-D-maltopyranoside, disodium PEG-4 cocamido MIPA-sulfosuccinate (“DMPS”), polysorbate 80 (PS-80) etc., fatty alcohols (cetyl alcohol or olelyl alcohol), or zwitterionic surfactants 3-(N,N-Dimethyltetradecylammonio)propanesulfonate (SB3-14), 3-(4-Heptyl)phenyl-3-hydroxypropyl)dimethylammoniopropanesulfonate (C7BzO), 3-(decyldimethylammonio) propanesulfonate inner salt (SB3-10), 3-(dodecyldimethylammonio) propanesulfonate inner salt (SB3-12), 3-(N,N-dimethyloctadecylammonio) propanesulfonate (SB3-18), 3-(N,N-dimethyl-octylammonio) propanesulfonate inner salt (SB3-8), 3-(N,N-dimethylpalmitylammonio) propanesulfonate (SB3-16), 3-[N,N-dimethyl(3-myristoylaminopropyl)ammonio]propane-sulfonate (ASB-14), CHAPS, CHAPSO, acetylated lecithin, alkyl(C12-30)dialkylamine-N-oxide apricotamidopropyl betaine, babassuamidopropyl betaine, behenyl betaine, bis 2-hydroxyethyl tallow glycinate, C12-14 alkyl dimethyl betaine, canolamidopropyl betaine, capric/caprylic amidopropyl betaine, capryloamidopropyl betaine, cetyl betaine, 3-[(Cocamidoethyl)dimethylammonio]-2-hydroxypropanesulfonate, 3-[(Cocamidoethyl)dimethyl-ammonio]propanesulfonate, cocamidopropyl betaine, cocamidopropyl dimethylamino-hydroxypropyl hydrolyzed collagen, N-[3-cocamido)-propyl]-N,N-dimethyl betaine, potassium salt, cocamidopropyl hydroxysultaine, cocamidopropyl sulfobetaine, cocaminobutyric acid, cocaminopropionic acid, cocoamphodipropionic acid, coco-betaine, cocodimethylammonium-3-sulfopropylbetaine, cocoiminodiglycinate, cocoiminodipropionate, coco/oleamidopropyl betaine, cocoyl sarcosinamide DEA, DEA-cocoamphodipropionate, dihydroxyethyl tallow glycinate, dimethicone propyl PG-betaine, N,N-dimethyl-N-lauric acid-amidopropyl-N-(3-sulfopropyl)-ammonium betaine, N,N-dimethyl-N-myristyl-N-(3-sulfopropyl)-ammonium betaine, N,N-dimethyl-N-palmityl-N-(3-sulfopropyl)-ammonium betaine, N,N-dimethyl-N-stearamidopropyl-N-(3-sulfopropyl)-ammonium betaine, N,N-dimethyl-N-stearyl-N-(3-sulfopropyl)-ammonium betaine, N,N-dimethyl-N-tallow-N-(3-sulfopropyl)-ammonium betaine, disodium caproamphodiacetate, disodium caproamphodipropionate, disodium capryloamphodiacetate, disodium capryloamphodipropionate, disodium cocoamphodiacetate, disodium cocoamphodipropionate, di sodium isostearoamphodipropionate, disodium laureth-5 carboxyamphodiacetate, disodium lauriminodipropionate, disodium lauroamphodiacetate, disodium lauroamphodipropionate, disodium octyl b-iminodipropionate, disodium oleoamphodiacetate, disodium oleoamphodipropionate, disodium PPG-2-isodeceth-7 carboxyamphodiacetate, disodium soyamphodiacetate, disodium stearoamphodiacetate, disodium tallamphodipropionate, disodium tallowamphodiacetate, disodium tallowiminodipropionate, disodium wheatgermamphodiacetate, N,N-distearyl-N-methyl-N-(3-sulfopropyl)-ammonium betaine, erucamidopropyl hydroxysultaine, ethylhexyl dipropionate, ethyl hydroxymethyl oleyl oxazoline, ethyl PEG-15 cocamine sulfate, hydrogenated lecithin, hydrolyzed protein, isostearamidopropyl betaine, 3-[(Lauramidoethyl)dimethylammonio]-2-hydroxypropanesulfonate, 3-[(Lauramidoethyl)dimethylammonio]propanesulfonate, lauramido-propyl betaine, lauramidopropyl dimethyl betaine, lauraminopropionic acid, lauroamphodipropionic acid, lauroyl lysine, lauryl betaine, lauryl hydroxysultaine, lauryl sultaine, linoleamidopropyl betaine, lysolecithin, milk lipid amidopropyl betaine, myristamidopropyl betaine, octyl dipropionate, octyliminodipropionate, n-octyl-N,N-dimethyl-3-ammonio-1-propanesulfonate, n-decyl-N,N-dimethyl-3-ammonio-1-propanesulfonate, n-dodecyl-N,N-dimethyl-3-ammonio-1-propane-sulfonate, n-tetradecyl-N,N-dimethyl-3-ammonio-1-propanesulfonate, n-hexadecyl-N,N-dimethyl-3-ammonio-1-propanesulfonate, n-octadecyl-N,N-dimethyl-3-ammonio-1-propane-sulfonate, oleamidopropyl betaine, oleyl betaine, 4,4(5H)-oxazoledimethanol, 2-(heptadecenyl) betaine, palmitamidopropyl betaine, palmitamine oxide, PMAL-C6, PMAL-C12, PMAL-C16, ricinoleamidopropyl betaine, ricinoleamidopropyl betaine/IPDI copolymer, sesamidopropyl betaine, sodium C12-15 alkoxypropyl iminodipropionate, sodium caproamphoacetate, sodium capryloamphoacetate, sodium capryloamphohydroxypropyl sulfonate, sodium capryloamphopropionate, sodium carboxymethyl tallow polypropylamine, sodium cocaminopropionate, sodium cocoamphoacetate, sodium cocoamphohydroxypropyl sulfonate, sodium cocoamphopropionate, sodium dicarboxyethyl cocophosphoethyl imidazoline, sodium hydrogenated tallow dimethyl glycinate, sodium isostearoamphopropionate, sodium lauriminodipropionate, sodium lauroamphoacetate, sodium oleoamphohydroxypropylsulfonate, sodium oleoamphopropionate, sodium stearoamphoacetate, sodium tallamphopropionate, soyamidopropyl betaine, stearyl betaine, 3-[(Stearamidoethyl)dimethylammonio]-2-hydroxypropanesulfonate, 3-[(Stearamidoethyl)-dimethylammonio]propanesulfonate, tallowamidopropyl hydroxysultaine, tallowamphopoly-carboxypropionic acid, trisodium lauroampho PG-acetate phosphate chloride, undecylenamidopropyl betaine, and wheat germamidopropyl betaine.

In some embodiments, the composition of the application consists essentially of virus, trehalose, carbohydrate, Tris and optionally albumin. In some embodiments, the composition of the application consists of over 95%, over 98%, over 99% or over 99.9% wt % virus, trehalose, carbohydrate, Tris and optionally albumin. As such, the composition of the application may comprise less than 0.1 wt % of surfactant and non-biobased polymeric excipients.

In some embodiments, the drying is carried out at two temperatures, first drying at about −50° C. to about 150° C. followed by second drying at a temperature of about 1° C. to about 25° C. In this drying method, compositions of the application may demonstrated increased titer loss at the first 6-8 weeks and then plateau in the titer loss for the remaining time of the 32 weeks at temperatures of 25 and 37° C. Thus, the method of the application may provide a composition that stabilizes the virus at 25 and 37° C. for at least 32 weeks.

In some embodiments, the drying is carried out at a temperature in the range of about 1° C. to about 150° C., optionally for 5-15 hours, followed directly or indirectly by drying at a higher temperature of about 150° C. to about 400° C., optionally for 5-15 hours. In some embodiments, the drying further comprises precooling of the sample before the drying, optionally for 15 min to about 1 hour.

In some embodiments, all drying stages are carried out at a temperature above 0° C. Temperatures used herein are temperature settings of a drying cabinet, for example the temperature of a shelf in a freeze dryer. During the drying step, temperature of the composition itself may drop because of evaporative cooling of the composition, and when a partially dry composition is obtained the temperature rises close to the set point temperature of the drying cabinet. Thus, the composition of the application is not frozen from a liquid prior to the application of a vacuum as in freeze drying. Storage stability of the dried product decreases with freeze drying in at least some compositions. Freeze drying may also result in dry product that is less stable when re-frozen during storage after drying. Optionally, temperature excursions of the composition itself below 0° C. (or other freezing temperature considering the applied pressure) caused by evaporative cooling of the composition are avoided, for example by maintaining suitable temperature and pressure during the drying process.

In some embodiments, the drying is carried out in a lyophilizer or freeze dryer, either of which may be used without freezing, or a foam dryer. In some embodiments, the drying is carried out under a pressure (in coordination with an appropriate temperature) sufficient to maintain the temperature of the composition below about 10° C. and above freezing point, for example at a vacuum with a pressure less than 100 mBar or less than 10 mBar. In some examples, the vacuum pressure is less than 200 pBar. In some embodiments, the drying is carried out at a constant pressure. In some embodiments, the drying is carried out at two different pressures. For example, first the drying is carried out under pressure of for example 20mBar and then under pressure of for example 125 uBar.

In some examples, the composition is placed in the vacuum as a bulk liquid rather than, for example, as a spray or thin film or as a frozen aqueous solid. In some embodiments, when the solvent is removed by evaporation, the method of the application produces a solid. A solid may have residual moisture. In some embodiments, when the composition is dried using foam drying (boiling), this method produces a dry product in the form of a foam. In these embodiments, the composition may be transformed from a solution into a dried foam structure in one step involving simultaneous boiling or foaming, and evaporation. In this embodiment, vacuum sufficient to foam the composition is used. For aliquots of about 100 uL, the drying time may be in the range of 15-25 hours including a stage of secondary drying after a dried or glass foam is produced. Longer drying times, for example up to 48 hours, may also be used but do not appear to materially improve stability. Other drying times may be used for aliquots of different volumes.

In some embodiments, the drying is carried out in a freeze dryer or other vacuum dryer operated at a temperature setpoint above 0° C.. In some examples, despite the temperature setting of the dryer, as the cabinet is evacuated the temperature in the vials may drop below 0° C. or other freezing point considering the applied pressure. However, by maintaining a balance between the temperature and the pressure of the cabinet, the temperature of the vial is optionally kept above the freezing point of the composition. Unless stated otherwise, temperatures used in examples refer to the temperature setting of the dryer and not necessarily the temperature in the vials or the temperature of the composition.

In some embodiments the composition is stored in a nitrogen-enhanced atmosphere. It has been shown that storage in nitrogen-enhanced atmosphere caused crystallization of the dried formulation of the application at a temperature of 37° C. with some buffers. The stability of the compositions stored in nitrogen-enhanced atmosphere can be improved by removing the free ions from the composition of the application. Thus, absence of CM buffer and NaCl may be preferable in some examples.

In some embodiments, the dried composition may be stored, for example at a temperature in the range of 1-55° C. or 1-40° C. or 4-25° C. In some embodiments, the virus is more stable in compositions stored at lower temperatures, for example 1-10° C. In some embodiments, compositions of the application, for example compositions comprising carbohydrate and trehalose, may preserve virus stability though temporary storage at a temperature below 0° C. In some embodiments, the composition of the application, for example the composition comprising trehalose and albumin and/or carbohydrate, preserves virus stability though temporary storage at a temperature above 0° C. In some embodiments, a composition of the application preserves virus stability at a temperature from about 2° C. to about 40° C., about 10° C. to about 30° C., or about 20° C. to about 25° C., or about 25° C. to about 40° C.. In some embodiments, composition of the application, preserves virus stability for at least 2 weeks, at least 3 months or at least 8 months at the above temperatures. In some embodiments, composition of the application, preserves virus stability for at least 4 days, or from 4 to 10 days at temperatures below freezing.

### III. Composition of the Application

The present application includes a composition comprising: a virus, trehalose, less than 10% water, a buffer, and optionally carbohydrate and/or albumin, for example at least 1% albumin by weight.

In some embodiments, the virus is a virus in any recombinant, derived or modified form. In some embodiments, the virus is a live-attenuated virus, an inactivated virus, a viral vector or a recombinant virus. In some embodiments, the virus is an RNA virus, for example an enveloped RNA virus. In some embodiments, the virus is a vesicular stomatitis virus (VSV). In some embodiments, the VSV is a recombinant VSV. In some embodiments, the virus is derived from an adenovirus. In some embodiments, the virus is formulated for administration in a biological preparation. In some embodiments, the virus is formulated for administration as a vaccine.

In some embodiments, the composition may provide a foamed glass (e.g. a sugar glass) encapsulating the virus. In some embodiments the composition may provide a non-foamed solid (e.g. a film) encapsulating the virus. In some embodiments, the composition contains at least 90% by weight of trehalose, carbohydrate and albumin.

In some embodiments, the buffer is any buffer that maintains the pH of the composition of the application within the range of 6.8 to 8.2. In some embodiments, the buffer is a CM buffer or a Tris buffer, for example a Tris-HCl buffer. The buffer may include 10-50 mM of Tris. In some embodiments, the buffer maintains the pH of the composition in the range of 6.9 to 8.1 or 7.2-7.5. In some embodiments, the buffer is present in the dry composition at a concentration of about 0.5 wt % to about 10 wt %. In some embodiments, the buffer is present in the dry composition at a concentration of about 1 wt %, about 1.5 wt %, about 2 wt %, about 4 wt %, or about 8 wt % and values therebetween.

In some embodiments, other buffers, for example a Histidine buffer, may be used. In some embodiments, the buffer does not contain substantial amounts of crystal forming components. For example, phosphate-buffered saline (PBS) might reduce the performance of the composition of the application. In some embodiments, CM buffer may produce sulfate crystals and accordingly a Tris-HCl buffer may be preferred over a CM buffer in some examples. In some embodiments, the composition has less than 1 wt % on a fully dried basis of sulphate and phosphate salts.

In some embodiments, the trehalose is present in the composition at a concentration of about 1.25% (w/v) to about 20% (w/v). In some embodiments, the trehalose is present in the composition at a concentration of about 1.25% (w/v), about 2.5% (w/v), about 5% (w/v), about 10% (w/v), or about 15% (w/v). Trehalose is available from a variety of commercial sources. In some embodiments, the trehalose is present in the dry composition in a concentration of about 30 wt % to about 70 wt %, or about 70 wt % to 99 wt %. In some embodiments, the trehalose is present in the dry composition in a concentration of about 35% wt %, about 35 wt %, about 40 wt %, about 45 wt %, about 50 wt %, about 55 wt %, about 60 wt %, about 65 wt %, or about 80 wt %, about 90 wt %, or about 95 wt % and values therebetween.

In some embodiments, the carbohydrate is selected from CMC, dextran or pullulan.

In some embodiments, the ratio of trehalose to carbohydrate is in the range of 4:1 to 0.5:1 by weight, for example about 2:1. In some embodiments, the ratio of trehalose to carbohydrate is about 1:1. In some embodiments, the ratio of trehalose to carbohydrate is about 3:1.

In some embodiments, the carbohydrate is present in the composition in a concentration of about 0.5% to about 15%. In some embodiments, the carbohydrate is present in the composition at a concentration of about 0.625%, about 1.25%, about 2.5%, about 5%, or about 10%. In some embodiments, the carbohydrate is present in the dry composition in a concentration of about 15% wt % to about 50 wt %. In some embodiments, the carbohydrate is present in the dry composition in a concentration of about 20 wt %, about 25 wt %, about 25 wt %, about 30 wt %, about 35 wt %, about 40 wt %, or about 45 wt % and values therebetween.

In some embodiments, the carbohydrate is pullulan. In some embodiments the pullulan has a molecular weight in the range of about 100,000 to about 200,000. ullulan having such molecular weights is commercially available.

Optionally, different ratios of pullulan to trehalose may be used. PT solutions may be viscous and can be prepared independently of a virus containing buffer and then added to the virus containing buffer. A ratio of trehalose to pullulan may be in the range of 0.5:1 to 9:1. In some embodiments, the ratio of trehalose to pullulan is about 2:1. In some embodiments, the ratio of trehalose to pullulan is about 1:1. In some embodiments, the ratio of trehalose to pullulan is about 3:1. The PT solution is optionally made by dissolving the pullulan and trehalose into an additional volume of the same buffer that is used in the virus containing buffer.

In some embodiments, the virus is grown in a fetal bovine serum free media. In some embodiments, when the virus stock has protein, buffer exchange removes substantial amounts of the protein. As such, in some embodiments, the virus stock after buffer exchange has minimal protein, typically less than 50 μg/mL, and is considered albumin free. However, albumin may be added to the composition of the application.

In some embodiments, the composition of the application comprises albumin. In some embodiments, the albumin can be added, for example as human serum albumin (HSA), bovine serum albumin (BSA) or a recombinant albumin such as recombinant human albumin. The pH of a composition may need to be adjusted to avoid a decrease in pH after adding albumin. In some embodiments, the concentration of the albumin in the composition before drying is in the range of 0.125%-2.5%. In some embodiments, the concentration of the albumin in the composition before drying is about 0.5% or about 2%. BSA is used in examples described herein for convenience. However, in a human vaccine HSA or a recombinant human albumin may be used. In an animal vaccine, a form of albumin acceptable to the animal may be used. In some embodiments, the ratio of trehalose to albumin is from 3:1 to 25:1. In some embodiments, the ratio of trehalose to albumin is 10:1. In some embodiments, the albumin is present in the dry composition in a concentration of at least 1 wt %, for example about 2 wt % to about 20 wt %. In some embodiments, the albumin is present in the dry composition in a concentration of about 2.5 wt %, about 4 wt %, about 7 wt %, or about 15 wt % and values therebetween.

In some embodiments, the composition of the application comprises NaCl. NaCl may be present in the composition at a concentration of 25-150 mM or 50-100 mM. NaCl may be present in the composition at a concentration of 50 mM. Compositions with higher concentrations of NaCl (for example 250 mM) may have increased residual moisture, process loss or reduced storage stability. In some embodiments, NaCl was found to be beneficial in combination with a CM buffer but might not be beneficial in combination with a Tris-HCl buffer. In some embodiments, the NaCl is present in the dry composition in a concentration of about 1 wt % to about 12 wt %. In some embodiments, the NaCl is present in the dry composition in a concentration of about 1.5 wt %, about 2 wt %, about 3 wt %, about 4 wt %, about 6 wt %, or about 10 wt % and values therebetween.

In some embodiments, the composition is dried from a liquid composition or reconstituted into a liquid composition.

In some embodiments, the composition is a dry composition. In some embodiments, the dry composition is prepared by the method of the application. In some embodiments, the dry composition is a foam. In some embodiments, the dry composition is a (non-foamed) solid.

In some embodiments, the dry composition of the application has a water content of about 1% to about 10%. As such, in some embodiments, the dry composition has a water content of less than 10 wt %. In some embodiments, the dry composition has a water content of less than 9 wt %. In some embodiments, the dry composition has a water content of less than 8 wt %. In some embodiments, the dry composition has a water content of less than 7 wt %. In some embodiments, the dry composition has a water content of less than 6 wt %. In some embodiments, the dry composition has a water content of less than 5 wt %. In some embodiments, the dry composition has a water content of about 1 wt % to about 10 wt %. In some embodiment, the dry composition a water content of about 1 wt % to about 9 wt %. In some embodiment, the dry composition has a water content of about 1 wt % to about 8 wt %. In some embodiment, the dry composition has a water content of about 1 wt % to about 7 wt %.

An exemplary composition and an optional range of parts by mass (excluding water) for other compositions are described in Table 1 or other tables below. In some embodiments, the dry composition comprises about 23 to about 300 parts of the trehalose. In some embodiments, the dry composition comprises about 1 to about 10 parts of the buffer. In some embodiments, the dry composition comprises about 12 to about 150 parts carbohydrate. In some embodiments, the dry composition comprises about 2 to about 35 parts of the albumin. In some embodiments, the dry composition comprises about 2 to about 12 parts of the NaCl. In some embodiments, the dry composition comprises about 0.0001 to about 0.01 parts of the virus.

In some embodiments, the dried composition may be stored, for example at a temperature in the range of 1-55° C. or 1-40° C. or 4-25° C. In some embodiments, the virus is more stable in compositions stored at lower temperatures, for example 1-10° C. In some embodiments, compositions of the application, for example compositions comprising pullulan and trehalose, may preserve virus stability though temporary storage at a temperature below 0° C. In some embodiments, the composition of the application, for example the composition comprising pullulan, trehalose and albumin, preserves virus stability though temporary storage at a temperature above 0° C. In some embodiments, the composition of the application preserves virus stability at a temperature from about 2° C. to about 40° C., about 10° C. to about 30° C., or about 20° C. to about 25° C., or about 25° C. to about 40° C.. In some embodiments, composition of the application, preserves virus stability for at least 2 weeks, at least 3 months or at least 8 months at the above temperatures. In some embodiments, composition of the application, preserves virus stability for at least 4 days, or from 4 to 10 days at temperatures below freezing.

In some embodiments, the composition of the application is used as, or as part of, or as a precursor of, a vaccine.

In some embodiments, the vaccine is for intramuscular, subcutaneous, intradermal, transdermal, oral, peroral, nasal, and/or inhalative application.

In some embodiments, the vaccine can be prepared by blending the composition of the application with one or more pharmaceutically acceptable excipients to generate a vaccine formulation. Exemplary pharmaceutically acceptable excipients for the purposes of pharmaceutical compositions disclosed herein include, but are not limited to, binders, disintegrants, superdisintegrants, lubricants, diluents, fillers, flavors, glidants, sorbents, solubilizers, chelating agents, emulsifiers, thickening agents, dispersants, suspending agents, adsorbents, granulating agents, buffers, coloring agents and sweeteners or combinations thereof.

It has been shown that further excipients such as polymers and amino acids such as PEG 200, PEG 4000, PEG 6000, Histidine, Glutamic acid, and polyvinylpyrrolidone such as polyvinylpyrrolidone K-15 (PVP K15) do not affect the stability of the composition of the application. As such, in some embodiments, the composition of the application is free from polymeric excipients. In some embodiments, the composition of the application is free from amino acids. The examples also showed that compositions with hydroxyectoine, ectoine, β-cyclodextran, ethylenediaminetetraacetic acid (EDTA) and sorbitol do not improve the stability.

The examples further showed that compositions with surfactants do not affect the stability of the composition of the application. In fact, it has been shown that composition with poly (Maleic Anhydride-Alt-1-Octadecene) substituted with 3-(Dimethylamino) Propylamine (PMAL-C16) lost titer after exchange of buffer into PMAL. As such, in some embodiments, the composition of the application is free from PMAL-C16.

In some embodiments, the composition of the application is free from high molecular weight surfactants such as Pluronic F127, Pluronic F68, Pluronic P123, or other EO-PO block copolymers of greater than 3,000-4,000 MW. In some embodiments, the composition of the application is free from any surfactants. Exemplary surfactants include, but are not limited to nonionic surfactants such as alkyl poly(ethylene oxide), copolymers of poly(ethylene oxide) and poly(propylene oxide) (EO-PO block copolymers), poly(vinyl pyrroloidone), alkyl polyglucosides (such as sucrose monostearate, lauryl diglucoside, or sorbitan monolaureate, octyl glucoside and decyl maltoside), hydroxypropyl methylcellulose (HPMC), poly(ethylene)glycol 3000, dodecyl-p-D-maltopyranoside, disodium PEG-4 cocamido MIPA-sulfosuccinate (“DMPS”), polysorbate 80 (PS-80) etc., fatty alcohols (cetyl alcohol or olelyl alcohol), or zwitterionic surfactants 3-(N,N-Dimethyltetradecylammonio)propanesulfonate (SB3-14), 3-(4-Heptyl)phenyl-3-hydroxypropyl)dimethylammoniopropanesulfonate (C7BzO), 3-(decyldimethylammonio) propanesulfonate inner salt (SB3-10), 3-(dodecyldimethylammonio) propanesulfonate inner salt (SB3-12), 3-(N,N-dimethyloctadecylammonio) propanesulfonate (SB3-18), 3-(N,N-dimethyl-octylammonio) propanesulfonate inner salt (SB3-8), 3-(N,N-dimethylpalmitylammonio) propanesulfonate (SB3-16), 3-[N,N-dimethyl(3-myristoylaminopropyl)ammonio]propane-sulfonate (ASB-14), CHAPS, CHAPSO, acetylated lecithin, alkyl(C12-30)dialkylamine-N-oxide apricotamidopropyl betaine, babassuamidopropyl betaine, behenyl betaine, bis 2-hydroxyethyl tallow glycinate, C12-14 alkyl dimethyl betaine, canolamidopropyl betaine, capric/caprylic amidopropyl betaine, capryloamidopropyl betaine, cetyl betaine, 3-[(Cocamidoethyl)dimethylammonio]-2-hydroxypropanesulfonate, 3-[(Cocamidoethyl)dimethyl-ammonio]propanesulfonate, cocamidopropyl betaine, cocamidopropyl dimethylamino-hydroxypropyl hydrolyzed collagen, N-[3-cocamido)-propyl]-N,N-dimethyl betaine, potassium salt, cocamidopropyl hydroxysultaine, cocamidopropyl sulfobetaine, cocaminobutyric acid, cocaminopropionic acid, cocoamphodipropionic acid, coco-betaine, cocodimethylammonium-3-sulfopropylbetaine, cocoiminodiglycinate, cocoiminodipropionate, coco/oleamidopropyl betaine, cocoyl sarcosinamide DEA, DEA-cocoamphodipropionate, dihydroxyethyl tallow glycinate, dimethicone propyl PG-betaine, N,N-dimethyl-N-lauric acid-amidopropyl-N-(3-sulfopropyl)-ammonium betaine, N,N-dimethyl-N-myristyl-N-(3-sulfopropyl)-ammonium betaine, N,N-dimethyl-N-palmityl-N-(3-sulfopropyl)-ammonium betaine, N,N-dimethyl-N-stearamidopropyl-N-(3-sulfopropyl)-ammonium betaine, N,N-dimethyl-N-stearyl-N-(3-sulfopropyl)-ammonium betaine, N,N-dimethyl-N-tallow-N-(3-sulfopropyl)-ammonium betaine, disodium caproamphodiacetate, disodium caproamphodipropionate, disodium capryloamphodiacetate, disodium capryloamphodipropionate, disodium cocoamphodiacetate, disodium cocoamphodipropionate, di sodium isostearoamphodipropionate, disodium laureth-5 carboxyamphodiacetate, disodium lauriminodipropionate, disodium lauroamphodiacetate, disodium lauroamphodipropionate, disodium octyl b-iminodipropionate, disodium oleoamphodiacetate, disodium oleoamphodipropionate, disodium PPG-2-isodeceth-7 carboxyamphodiacetate, disodium soyamphodiacetate, disodium stearoamphodiacetate, disodium tallamphodipropionate, disodium tallowamphodiacetate, disodium tallowiminodipropionate, disodium wheatgermamphodiacetate, N,N-distearyl-N-methyl-N-(3-sulfopropyl)-ammonium betaine, erucamidopropyl hydroxysultaine, ethylhexyl dipropionate, ethyl hydroxymethyl oleyl oxazoline, ethyl PEG-15 cocamine sulfate, hydrogenated lecithin, hydrolyzed protein, isostearamidopropyl betaine, 3-[(Lauramidoethyl)dimethylammonio]-2-hydroxypropanesulfonate, 3-[(Lauramidoethyl)dimethylammonio]propanesulfonate, lauramido-propyl betaine, lauramidopropyl dimethyl betaine, lauraminopropionic acid, lauroamphodipropionic acid, lauroyl lysine, lauryl betaine, lauryl hydroxysultaine, lauryl sultaine, linoleamidopropyl betaine, lysolecithin, milk lipid amidopropyl betaine, myristamidopropyl betaine, octyl dipropionate, octyliminodipropionate, n-octyl-N,N-dimethyl-3-ammonio-1-propanesulfonate, n-decyl-N,N-dimethyl-3-ammonio-1-propanesulfonate, n-dodecyl-N,N-dimethyl-3-ammonio-1-propane-sulfonate, n-tetradecyl-N,N-dimethyl-3-ammonio-1-propanesulfonate, n-hexadecyl-N,N-dimethyl-3-ammonio-1-propanesulfonate, n-octadecyl-N,N-dimethyl-3-ammonio-1-propane-sulfonate, oleamidopropyl betaine, oleyl betaine, 4,4(5H)-oxazoledimethanol, 2-(heptadecenyl) betaine, palmitamidopropyl betaine, palmitamine oxide, PMAL-C6, PMAL-C12, PMAL-C16, ricinoleamidopropyl betaine, ricinoleamidopropyl betaine/IPDI copolymer, sesamidopropyl betaine, sodium C12-15 alkoxypropyl iminodipropionate, sodium caproamphoacetate, sodium capryloamphoacetate, sodium capryloamphohydroxypropyl sulfonate, sodium capryloamphopropionate, sodium carboxymethyl tallow polypropylamine, sodium cocaminopropionate, sodium cocoamphoacetate, sodium cocoamphohydroxypropyl sulfonate, sodium cocoamphopropionate, sodium dicarboxyethyl cocophosphoethyl imidazoline, sodium hydrogenated tallow dimethyl glycinate, sodium isostearoamphopropionate, sodium lauriminodipropionate, sodium lauroamphoacetate, sodium oleoamphohydroxypropylsulfonate, sodium oleoamphopropionate, sodium stearoamphoacetate, sodium tallamphopropionate, soyamidopropyl betaine, stearyl betaine, 3-[(Stearamidoethyl)dimethylammonio]-2-hydroxypropanesulfonate, 3-[(Stearamidoethyl)-dimethylammonio]propanesulfonate, tallowamidopropyl hydroxysultaine, tallowamphopoly-carboxypropionic acid, trisodium lauroampho PG-acetate phosphate chloride, undecylenamidopropyl betaine, and wheat germamidopropyl betaine.

In some embodiments, the composition of the application consists essentially of virus, trehalose, carbohydrate, Tris and optionally albumin. In some embodiments, the composition of the application consists of over 99.9% wt % virus, trehalose, carbohydrate, Tris and optionally albumin. As such, the composition of the application comprises less than 0.1 wt % of surfactant and non-biobased polymeric excipients.

TABLE 1

| | | | | | Total | | Range |
|---|---|---|---|---|---|---|---|
| | | | | | mass | Percent | in Parts |
| Component | Concentration | Vol | MW | ug/100 uL | (ug) | Film | by Mass |
| Tris-HCl | 10 mM | 100 | 121.14 | | 121.14 | 1.533 | 1-10 |
| MgS04 | 10 mM | 100 | 120.336 | | 120.336 | 1.523 | 0-5 |
| CaCl2 | 10 mM | 100 | 110.98 | | 110.98 | 1.405 | 0-5 |
| Gelatin | 0.005% | 100 | | 5 | 5 | 0.063 | 0-2 |
| Albumin | 0.50% | 100 | | 500 | 500 | 6.329 | 2-35 |
| NaCl | 50 mM | 100 | 58.44 | | 292.2 | 3.699 | 2-12 |
| Pullulan | 2.50% | 90 | | 2500 | 2250 | 28.482 | 0-150 |
| Trehalose | 5% | 90 | | 5000 | 4500 | 56.964 | 25-300 |
| Virus | 1.00E+08 | | 265 mDa | | 0.04 | 0.001 | 0001-.01 |
| Total | | | | | 7899.7 | 100 | |

US Patent Application Publication No. US 2019/0111006 A1, Method of Long-Term Preservation of Chemical and Biological Species Using Sugar Glasses, is incorporated herein in its entirety by this reference.

In the examples presented below, modified reporter viruses are used as a model to represent recombinant viruses that would be used in viral vector vaccines, in particular VSV or AdV vectored vaccines. The VSV are Indiana serotype (e.g. VSV-XN GFP), replicating and have modification to produce green fluorescent protein (GFP) (e.g. VSV-XN GFP) or a red fluorescent protein (mCherry). The AdV are non-replicating human adenovirus serotype 5 with modifications to delete the E1/E3 genes and to produce GFP (huAd5-GFP). Considering the dilutions in the assays, results are typically considered to be +/−0.25 log unless indicated otherwise.

### EXAMPLES

### Example 1: Formulation Preparation

### Example 1a: Tris/Gelatin/Sorbitol Formulation with Vesicular Stomatitis Virus—green fluorescent protein (VSV-GFP)

VSV-GFP was transferred from an initial commercial sample to a buffer solution by way of buffer exchange using a spin column to create a stock solution. The buffer was a tris(hydroxymethyl)aminomethane (Tris) buffer (10 mM) with a pH of 7.2. A storage solution of 0.5% gelatin and 2% sorbitol mixed into the same Tris buffer was prepared. 90 μL of the storage solution was mixed with 10 μL of the stock solution to create 100 μL aliquots having an initial titer of about 10¹ PFU.

### Example 1b: Pullulan/Trehalose/CM Formulation with VSV-mCherry

VSV-mCherry was transferred from an initial commercial sample to a buffer solution by way of buffer exchange using a spin column to create a stock solution. The buffer was a CM buffer with a pH of 7.2. A storage solution of 5% pullulan and 10% trehalose mixed into the same CM buffer was prepared. 90 μL of the storage solution was mixed with 10 μL of the stock solution to create 100 μL aliquots having an initial titer of about 10¹ PFU. VSV-mCherry had minimal albumin carryover of about 10 pg, or about 0.01% of the storage solution.

### Example 1c: Pullulan/Trehalose/CM Formulation with VSV-GFP

VSV-GFP was transferred from an initial commercial sample to a buffer solution by way of buffer exchange using a spin column to create a stock solution. The buffer was a CM buffer with a pH of 7.2. A storage solution of 5% pullulan and 10% trehalose mixed into the same CM buffer was prepared. 90 μL of the storage solution where mixed with 10 μL of the stock solution to create 100 μL aliquots having an initial titer of about 10¹ PFU. VSV-GFP had albumin carryover of about 60 pg, or about 0.05% of the storage solution.

### Example 1d: Pullulan/Trehalose/CM/Albumin Formulation with VSV-mCherry and albumin

VSV-mCherry was transferred from an initial commercial sample to a buffer solution by way of buffer exchange using a spin column to create a stock solution. The buffer was a CM buffer with a pH of 7.2. A storage solution of 2.5% pullulan, 5% trehalose and 0.5% albumin mixed into the same CM buffer was prepared. 90 μL of the storage solution was mixed with 10 μL of the stock solution to create 100 μL aliquots having an initial titer of about 10¹ PFU.

### Example 1e:Pullulan/Trehalose/CM/BSA/NaCl Formulation with VSV-GFP

VSV-GFP was transferred from an initial commercial sample to a buffer solution by way of buffer exchange using a spin column to create a stock solution. The buffer was a CM buffer modified by adding 0.5% bovine serum albumin (BSA) and 50 mM NaCl with a pH of 7.2. A storage solution of 2.5% pullulan and 5% trehalose mixed into the same modified CM buffer was prepared. 90 μL of the storage solution was mixed with 10 μL of the stock solution to create 100 μL aliquots having an initial titer of about 10¹ PFU.

### Example If: Tris/Trehalose Formulation with VSV-GFP

VSV-GFP was transferred from an initial commercial sample to a buffer solution by way of buffer exchange using a spin column to create a stock solution. The buffer was a Tris buffer (10 mM) with a pH of 7.2. A storage solution of 5% trehalose mixed into the same Tris buffer was prepared. 90 μL of the storage solution was mixed with 10 μL of the stock solution to create 100 μL aliquots having an initial titer of about 10¹ PFU.

### Example 2:Tris/Gelatin/Sorbitol Composition with VSV-GFP dried at 20° C.

Aliquots of the composition of Example 1a were dried at 20° C. for 21 hours. Aliquots dried at 200 pBar (A) had an average residual moisture (RM) of 2.3%. Aliquots dried at 16 pBar (B) had an average residual moisture (RM) of 1.1%.

Aliquots A and B both experienced a process loss in titer of about 1 log, as determined by a plaque assay performed directly after drying. There were no detectable PFU after storing the dried composition for 7 days at 37° C.

As indicated by these results, the combination of Tris, gelatin and sorbitol was not effective at stabilizing the composition containing VSV.

### Example 3: Compositions of Examples 1b, 1c and 1d dried at 20° C.

Aliquots of the composition of Examples 1b, 1c and 1d were dried at 20° C. under vacuum for 24 hours and stored at 37° C. Process loss and titer loss (including process loss) at 7 and 18 days is described in Table 1 below.

TABLE 2
Process and Titer Loss

| Composition | Example 1b | Example 1c | Example 1d |
|---|---|---|---|
| Process loss (log) | 2.0 | 1.5 | 1.0 |
| Titer Loss Day 7 | 2.5 | 2.1 | 1.7 |
| (log) | | | |
| Titer Loss Day 18 | 4 | 2.5 | 2.1 |
| (log) | | | |

### Example 4: PT/CM/BSA/NaCl Composition with VSV-GFP Dried at 20° C.

Aliquots of the composition in Example 1e were dried at 20° C. for 21 hours. Aliquots dried at 200 pBar (A) had an average residual moisture (RM) of 3.9%. Aliquots dried at 16 pBar (B) had an average residual moisture (RM) of 3.5%.

Aliquots A experienced an average process loss in titer of 1.3 log. Aliquots B experienced an average process loss in titer of about 1 log.

Aliquots B were stored at 37° C. Plaque assays performed at 7 and 14 days indicated a loss in titer (relative to the initial 10¹ PFU sample titer) of about 2 log at both times. Plaque assays performed at 28 days indicated a loss in titer (relative to the initial 10¹ PFU sample titer) of about 3 log.

### Example 5: Tris/Gelatin/Sorbitol Composition with VSV-GFP Dried at 4° C.)

Aliquots of the composition of Example 1a were dried for 21 hours at 4° C. and about 12-15 pBar. The average residual moisture (RM) of the dried compositions was 0.7%. Drying produced a process loss in titer of about 0.3 log, as determined by a plaque assay.

Aliquots of the composition were stored at 37° C. Plaque assays performed at 7 and 14 days indicated losses in titer (relative to the initial 10¹ PFU sample titer) of about 5.4 log and 8 log respectively.

### Example 6: Pullulan/Trehalose(PT)/CM/BSA/NaCl Composition with VSV-GFP Dried at 4° C.

Aliquots of the composition of Example 1e were dried for 21 hours at 4° C. and about 12-15 pBar. The average residual moisture (RM) of the dried compositions was 8.6%. Drying produced a process loss in titer of about 0.4 log, as determined by a plaque assay.

Aliquots of the composition were stored at 37° C. Plaque assays performed at 7 and 14 days indicated losses in titer (relative to the initial 10¹ PFU sample titer) of about 2.5 log and 3 log respectively.

### Example 7: PT/CM/BSA/NaCl Composition with VSV-GFP Dried at 4° C./25° C.

Aliquots of the composition of Example 1e were dried for 21 hours at about 15 pBar. The 21 hours of drying included 10 hours at 4° C., followed by a temperature ramping up from 4° C. to 25° C. over 5 hours, followed by drying for 6 hours at 25° C.. The average residual moisture (RM) of the dried compositions was 6.1%. Drying produced no detectable process loss, as determined by a plaque assay.

Aliquots of the composition were stored at 37° C. Plaque assays performed at 7 and 14 days indicated losses in titer (relative to the initial 10¹ PFU sample titer) of about 1.2 log and 1.8 log respectively.

### Example 8: Tris/Trehalose Composition with VSV-GFP Dried at 4° C./25° C.

Aliquots of the composition of Example 1f were dried for 21 hours at about 15 pBar. The 21 hours of drying included 10 hours at 4° C., followed by a temperature ramping up from 4° C. to 25° C. over 5 hours, followed by drying for 6 hours at 25° C. The average residual moisture (RM) of the dried compositions was 3.5%. Drying produced a process loss in titer of about 0.5 log, as determined by a plaque assay.

Aliquots of the composition were stored at 37° C. Plaque assays performed at 7 and 14 days indicated losses in titer (relative to the initial 10¹ PFU sample titer) of about 1.1 log and 1.2 log respectively.

### Example 9: PT/CM/BSA/NaCl Composition with VSV-GFP Dried at −50° C./25° C.)

Aliquots of the composition of Example 1e were dried for 21 hours at about 15 pBar. The 21 hours of drying included 10 hours at −50° C., followed by a temperature ramping up from −50° C. to 25° C. over 6 hours, followed by drying for 5 hours at 25° C. The average residual moisture (RM) of the dried compositions was 1.7%. Drying produced about 0.5 log process loss, as determined by a plaque assay.

Aliquots of the composition were stored at 37° C. A plaque assays performed at 7 days indicated a loss in titer (relative to the initial 10¹ PFU sample titer) of about 3 log.

### Example 10: Tris/Trehalose Composition with VSV-GFP Dried at −50° C./25° C.)

Aliquots of the composition of Example 1f were dried for 21 hours at about 15 pBar. The 21 hours of drying included 10 hours at −50° C., followed by a temperature ramping up from −50° C. to 25° C. over 6 hours, followed by drying for 5 hours at 25° C. The average residual moisture (RM) of the dried compositions was 1.0%. Drying produced a process loss in titer of about 1 log, as determined by a plaque assay.

Aliquots of the composition were stored at 37° C. A plaque assay performed at 7 days indicated a loss in titer (relative to the initial 10¹ PFU sample titer) of about 8 log.

### Example 11: PT/CM/BSA/NaCl Composition with VSV-GFP Dried at 4° C./37° C.)

Aliquots of the composition of Example 1e were dried for 21 hours at about 15 pBar. The 21 hours of drying included 10 hours at 4° C., followed by a temperature ramping up from 4° C. to 37° C. over 6 hours, followed by drying for 6 hours at 37° C.. The average residual moisture (RM) of the dried compositions was about 5.7%. Drying produced a process loss of about 0.5 log.

Aliquots of the composition were stored at 37° C. Plaque assay performed at 7 days indicated loss in titer (relative to the initial 10¹ PFU sample titer) of about 1.5 log. Plaque assay performed at 14 days indicated loss in titer (relative to the initial 10¹ PFU sample titer) of about 1.9 log.

### Example 12—PT/CM/BSA/NaCl Composition Dried at 20° C.

VSV-GFP was transferred from an initial solution to a dialysis buffer solution by way of buffer exchange using Zeba spin column to create two stock solutions.

Two formulations were prepared using 10% by volume of stock solution and 90% by volume of a solution of either a) pullulan and trehalose or b) gelatin and sorbitol, each dissolved in one of the dialysis buffers, as indicated in Table 3 below.

TABLE 3
Formulations for the Study

| | E-a | Control |
|---|---|---|
| VSV Stock | GFP (Lot 354) | |
| Dialysis Buffer (pH 7.2) | CM + 0.5% BSA + 50 mM | Tris |
| | NaCl | 0.5% Gelatin + |
| Formulation (Dialysis | 2.5% pullulan and | 2% Sorbitol |
| Buffer Base) | 0.125M (5%) trehalose | |

| | |
|---|---|
| Total Volume | 100 |

100 μL aliquots of the samples were foam dried under vacuum in 2 mL glass vials for 21 hours at 20° C. Some of the samples were dried in a dessicator at a pressure setpoint of 200 pBar. Other samples were dried with freeze dryer at a pressure setpoint of 16 pBar. No statistically significant difference in stability was detected between samples dried at the different pressures. After drying, samples were stoppered and crimped.

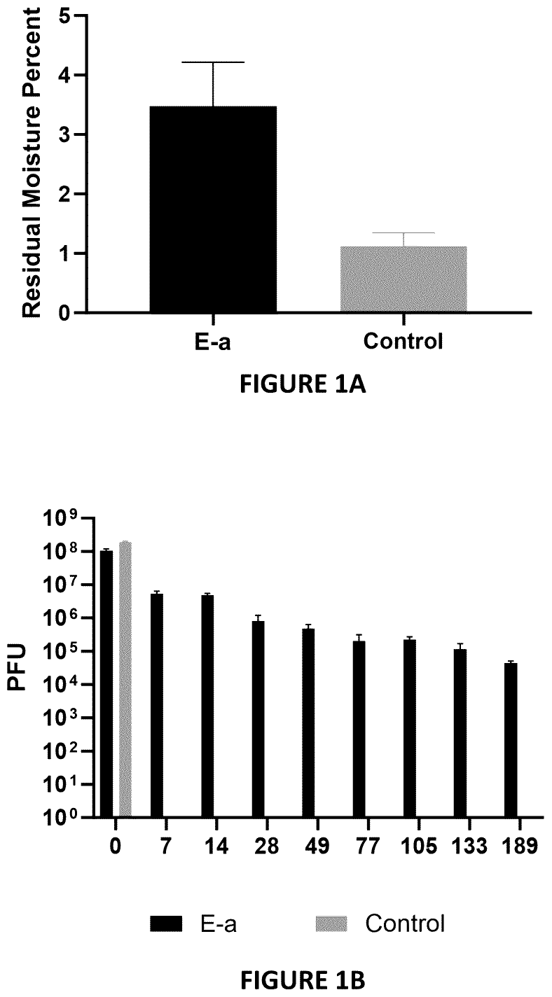

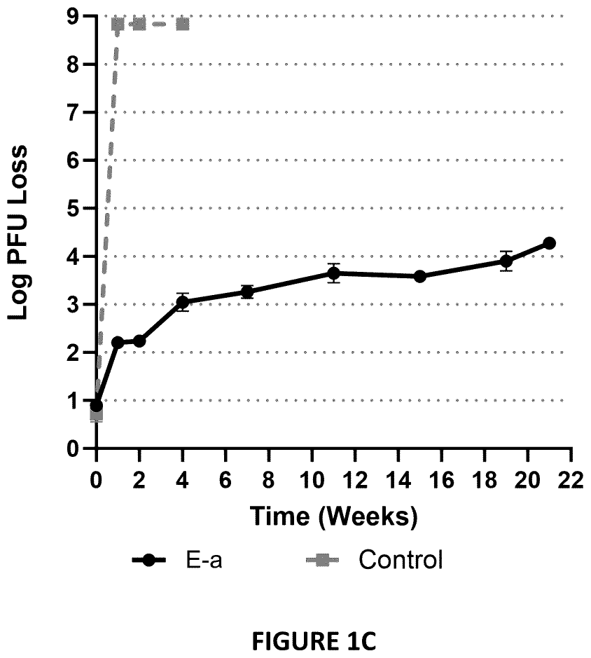

The residual moisture (RM) of the E-a formulation was about 3.5%. The residual moisture of the control (comparative) formulation was about 1.1% (FIG. 1A). There was less than 1 log of process loss for both formulation samples

The samples were incubated at 37° C. for 22 and 27 weeks (FIGS. 1B and 1C). The control formulation had no detectable PFU after 7 days of incubation at 37° C. E-a formulation showed about 2.5 log total loss (2×10⁵ PFU) at week 15 and about 4.3 log total loss at week 27. Table 4 below shows the wt % of the components in the dry formulation.

TABLE 4

| | |
|---|---|
| E-a | E-a |
| (As made | Wt % Dried |
| Formulation) | Formulation |

| | | | |
|---|---|---|---|
| Pullulan | 2.5 | wt % | 28.5 |
| Trehalose | 5 | wt % | 57.0 |
| BSA | 0.5 | wt % | 6.3 |
| NaCl | 50 | mM | 3.7 |
| Tris | 10 | mM | 1.5 |
| Gelatin | 0.01 | wt % | 0.1 |
| MgSO4 | 10 | mM | 1.5 |
| CaCl2 | 10 | mM | 1.4 |
| Virus | 1e8 | particles | 0.001 |

### Example 13: PT/CM/BSA/NaCl Composition Dried at 4° C./25° C.

VSV-GFP was transferred from an initial solution to a dialysis buffer solution by way of buffer exchange using Zeba spin column (0.5 mL columns) to create two stock solutions.

Two formulations were prepared using 10% by volume of stock solution and 90% by volume of a solution of either a) pullulan and trehalose or b) trehalose, each dissolved in one of the dialysis buffers, as indicated in Table 4 below.

TABLE 4
Formulations for The Study

| | E-b | Trehalose |
|---|---|---|
| VSV Stock | GFP (Lot 356) | |
| Dialysis Buffer (pH 7.2) | CM + 0.5% BSA + | Tris |
| | 50 mM NaCl | |
| Formulation (+Dialysis Buffer) | 2.5% Pullulan | 5% Trehalose |
| | 0.125M (5%) | |
| | Trehalose | |

| | |
|---|---|
| Total Volume | 100 |

100 μL aliquots of the samples were foam dried in freeze dryer in 2 mL glass vials for 21 hours using a two-stage drying protocol. The first (primary) drying stage was 10 hours at 4° C. The second stage was 4.5 hours at 25° C.. Between the first and second stage, the temperature ramped from 4° C. −25° C. over 6.5 hours. The pressure setpoint during the entire 21 hours was about 16 pBar. After drying, samples were stoppered and crimped.

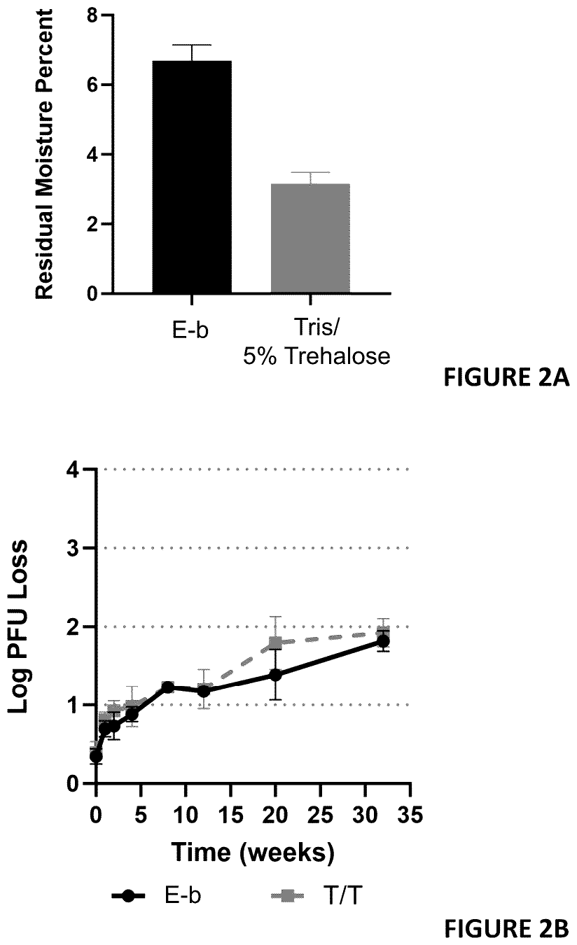

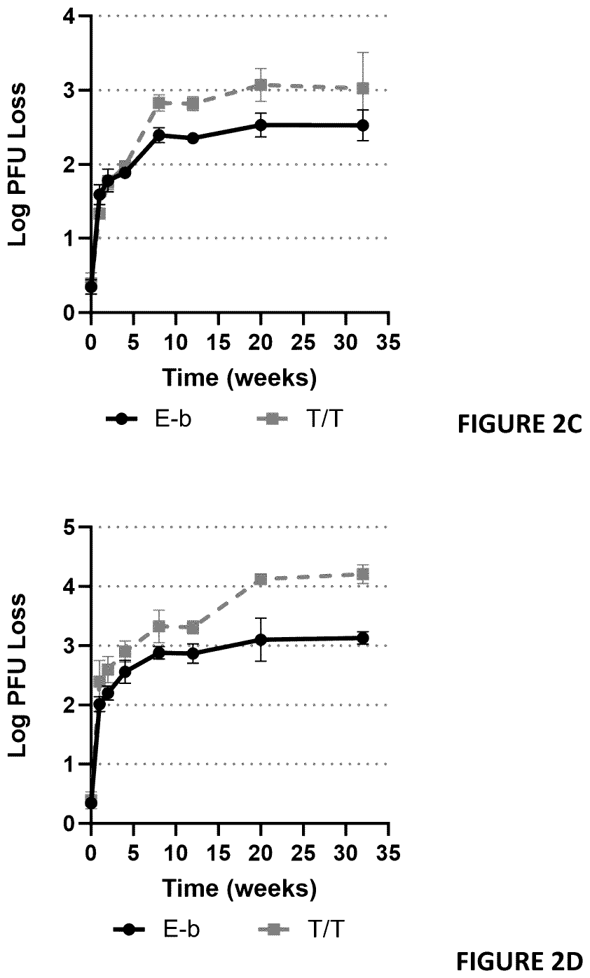

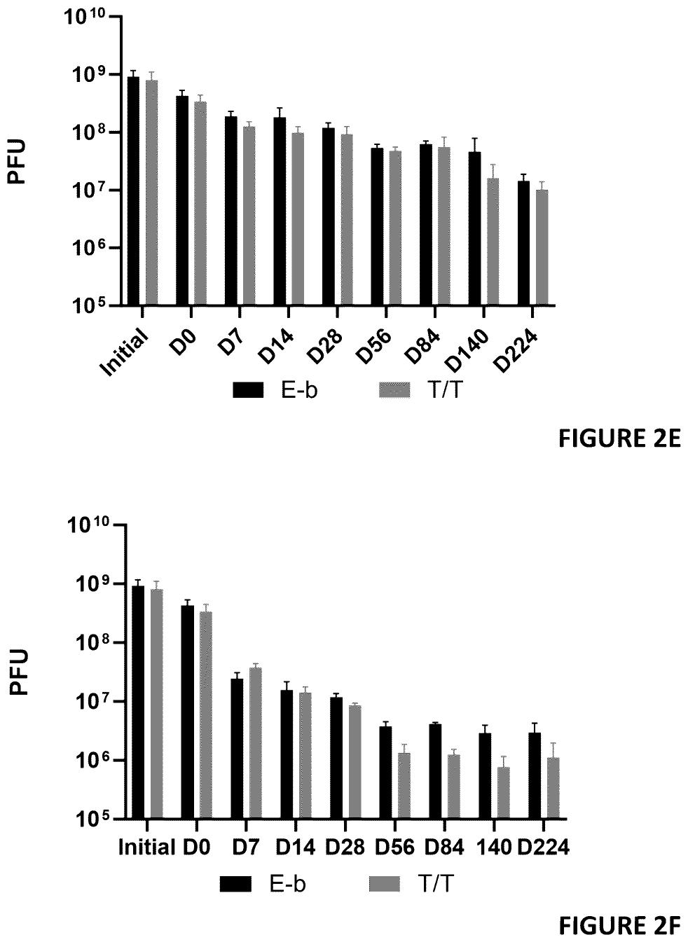

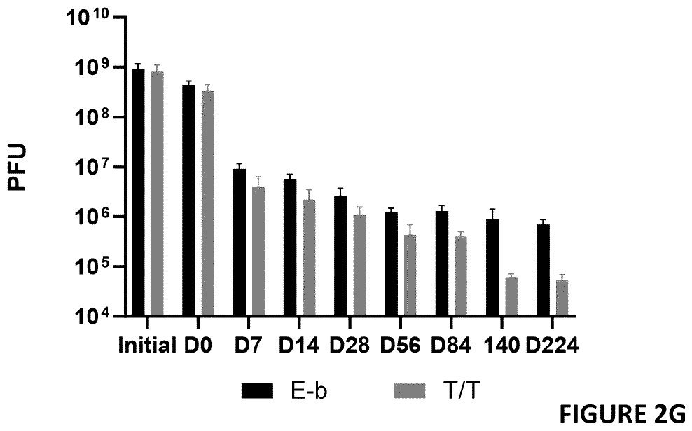

The residual moisture (RM) of the E-b formulation was about 6.7%. The residual moisture of the Trehalose formulation was about 3.2% (FIG. 2A). There was less than 1 log of process loss for both samples.

The samples were incubated at 4° C., 25° C. and 37° C. for 32 weeks. FIGS. 2B-G present the results of this study. The results show similar results in E-b and Trehalose formulations in the 4° C. incubated samples. E-b formulation showed less total loss at week 32 at higher temperatures. Table 5 below shows the wt % of the components in the dry formulation.

TABLE 5

| | E-b | | E-b | Tris/Trehalose | | Tris/Trehalose |
|---|---|---|---|---|---|---|
| | (As made | | Wt % Dried | (As Made | | (Wt % Dried |
| | Formulation) | | Formulation | Formulation) | | Formulation) |
| Pullulan | 2.5 | wt % | 28.5 | — | | — |
| Trehalose | 5 | wt % | 57.0 | 5 | wt % | 97.4 |
| BSA | 0.5 | wt % | 6.3 | — | | — |
| NaCl | 50 | mM | 3.7 | — | | — |
| Tris | 10 | mM | 1.5 | 10 | mM | 2.5 |

| | | | | | |
|---|---|---|---|---|---|
| Gelatin | 0.01 | wt % | 0.1 | — | — |
| MgSO4 | 10 | mM | 1.5 | — | — |
| CaCl2 | 10 | mM | 1.4 | — | — |

| | | | | |
|---|---|---|---|---|
| Virus | 1e8 | 0.001 | 1e8 | 0.001 |
| | particles | | particles | |

### Example 14: PT/CM/BSA/NaCl Composition in FBS free Media Dried at 4° C./25° C. (Sprint 19)

In the examples described above, VSV-GFP stock was grown in fetal bovine serum (FBS). After buffer exchange some albumin carried over into the VSV stock. In this example and the examples described below, VSV-GFP is grown under GMP conditions in FBS free media. While there is still some protein in the VSV stock, after buffer exchange the stock solutions have minimal protein, typically less than 50 μg/mL. Formulations made from these stock solutions are considered albumin free unless albumin is added.

FBS free VSV-GFP was transferred from an initial solution to a dialysis buffer solution by way of buffer exchange using a Zeba spin column (0.5 mL columns) to create two stock solutions.

Four formulations were prepared using 10% by volume of stock solution and 90% by volume of a formulation solution, each dissolved in one of the dialysis buffers, as indicated in Table 6 below.

TABLE 6
Formulations for The Study

| | E-c1 | E-c1/−BSA | Trehalose + BSA | Trehalose − BSA |
|---|---|---|---|---|
| VSV Stock | GFP (Lot 357) | | | |
| Dialysis Buffer (pH | CM + 50 mM NaCl + | CM + 50 mM NaCl | Tris + 0.5% BSA | Tris |
| 7.2) 10u: | 0.5% BSA | | | |
| Formulation | 2.5% Pullulan | 2.5% Pullulan | 5% Trehalose | 5% Trehalose |
| (+Dialysis Buffer) | 5% (0.125M) | 5% (0.125M) | | |
| 90 uL | Trehalose | Trehalose | | |

| | |
|---|---|
| Total Volume | 100 uL |

100 μL aliquots of the samples were foam dried in freeze dryer in 2 mL glass vials for 21 hours using a two-stage drying protocol. The first (primary) drying stage was 10 hours at 4° C. The second stage was 4.5 hours at 25° C.. Between the first and second stage, the temperature ramped from 4-25° C. over 6.5 hours. The samples were pre-cooled to 4° C. for 30 minutes before starting the drying. The pressure setpoint during the entire 21 hours was about 16 pBar. After drying, samples were stoppered and crimped.

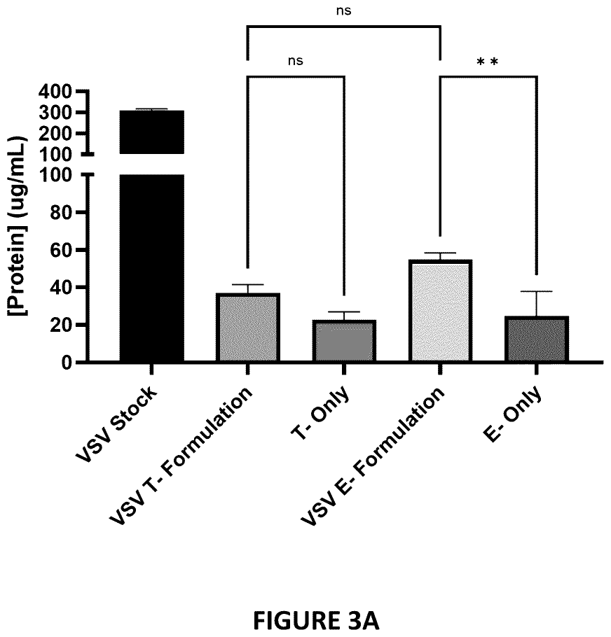

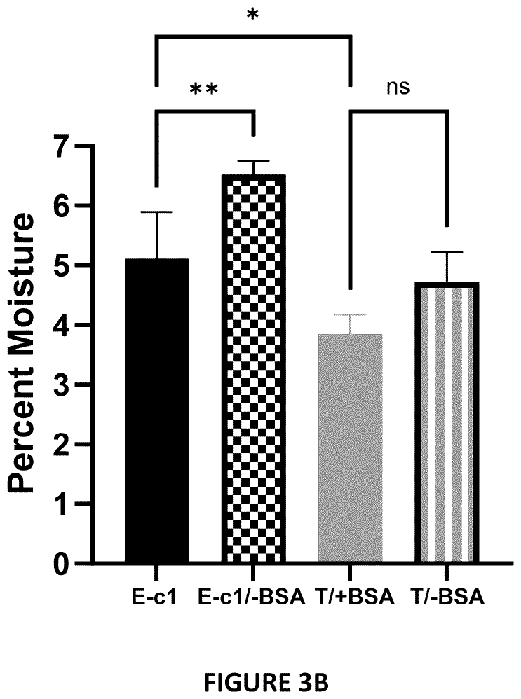

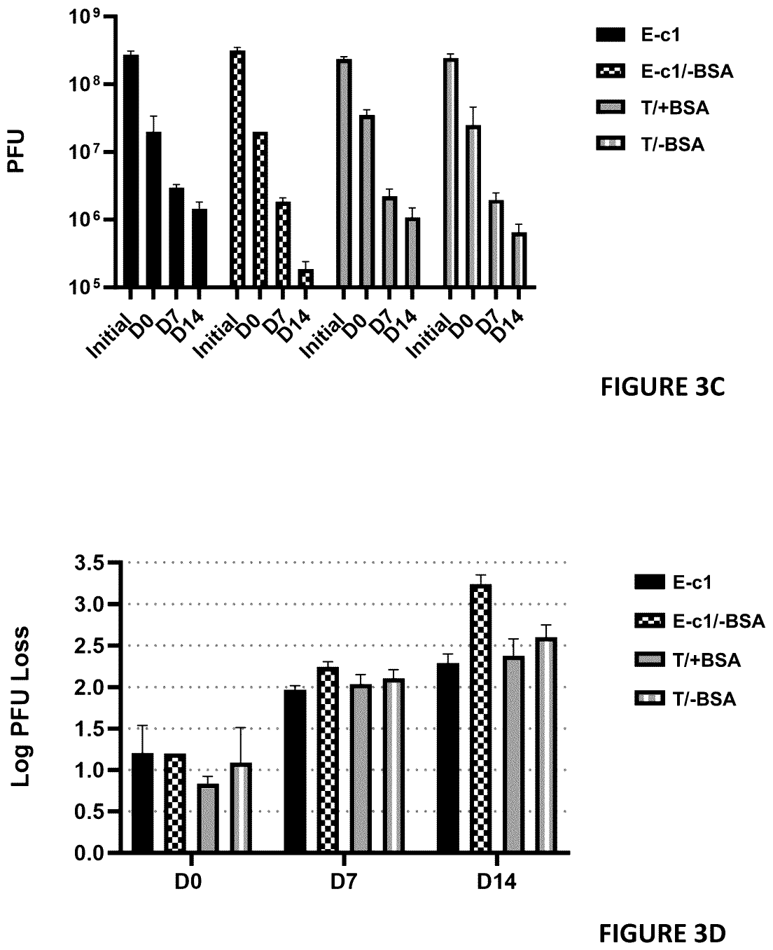

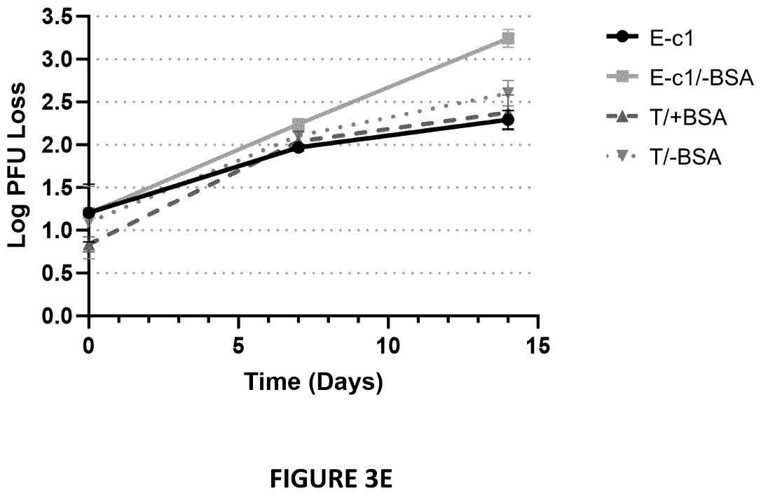

The samples were analyzed to quantify total protein using Bradford analysis and the results are shown in FIG. 3A. Background protein was detected in both buffers alone (T-Only and E-Only). Protein levels in dialyzed (buffer-exchanged) formulations (VSV T-Formulation and VSV E-Formulation) had insignificant protein levels.

The residual moisture (RM) of the E-c formulations was about 5% with BSA added and 6% without BSA. The residual moisture of the Trehalose formulation was about 4% with BSA and 5% without BSA. FIG. 3B presents the results.

The samples were incubated at 37° C. for 14 days. FIGS. 3C-3E presents the results of this study. The results showed no difference in titer between formulations post dialysis. Pre-dialysis calculated titer was 2.3e10 PFU/mL. Post dialysis for all samples was −2.5e10 PFU/mL. The results show improvement in stability in formulations with albumin. Table 7 below shows the wt % of the components in the dry formulation.

TABLE 7

| | | | | Elarex Lead − | | Elarex Lead − | Tris/Trehalose + | | Tris/Trehalose + | | | |
|---|---|---|---|---|---|---|---|---|---|---|---|---|
| | Elarex Lead | | Elarex Lead | BSA | | BSA | BSA | | BSA | Tris/Trehalose | | Tris/Trehalose |
| | (As made | | Wt % Dried | (As made | | Wt % Dried | (As made | | Wt % Dried | (As made | | (Wt % Dried |
| | Formulation) | | Formulation | Formulation) | | Formulation | Formulation) | | Formulation | Formulation) | | Formulation) |
| Pullulan | 2.5 | wt % | 28.5 | 2.5 | wt % | 30.4 | — | | — | — | | — |
| Trehalose | 5 | wt % | 57.0 | 5 | wt % | 60.8 | 5 | wt % | 87.9 | 5 | wt % | 97.4 |
| BSA | 0.5 | wt % | 6.3 | — | | — | 0.5 | wt % | 9.8 | — | | — |
| NaCl | 50 | mM | 3.7 | 50 | mM | 3.9 | — | | — | — | | — |
| Tris | 10 | mM | 1.5 | 10 | mM | 1.6 | 10 | mM | 2.4 | 10 | mM | 2.5 |

| | | | | | | | | | | |
|---|---|---|---|---|---|---|---|---|---|---|
| Gelatin | 0.01 | wt % | 0.1 | 0.01 | wt % | 0.1 | — | — | — | — |
| MgSO4 | 10 | mM | 1.5 | 10 | mM | 1.6 | — | — | — | — |
| CACl2 | 10 | mM | 1.4 | 10 | mM | 1.5 | — | — | — | — |

| | | | | | | | | |
|---|---|---|---|---|---|---|---|---|
| Virus | 1e8 particles | 0.001 | 1e8 particles | 0.001 | 1e8 particles | 0.001 | 1e8 particles | 0.001 |

### Example 15: PT/CM/BSA/NaCl Composition in FBS free Media Dried at 4° C./25° C. With 0.5%/2% BSA

FBS free VSV-GFP was transferred from an initial solution to a dialysis buffer solution by way of buffer exchange using a Zeba spin column (0.5 mL columns) to create stock solutions.

Eight formulations were prepared using 10% by volume of stock solution and 90% by volume of a formulation solution, each dissolved in one of the dialysis buffers, as indicated in Table 6 below.

TABLE 6
Formulations for The Study

| | E-c1 | E-c2 | E-c3 | E-c4 | E-c5 | E-c6 | E-c7 | E-c8 |
|---|---|---|---|---|---|---|---|---|
| VSV Stock | GFP (Lot 357) | | | | | | | |
| Dialysis Buffer | CM + 0.5%BSA + 50 mM NaCl | | | | CM + 2%BSA + 50 mM NaCl | | | |
| Formulation | 2.5% P | 5% P | 5% P | 10% P | 2.5% P | 5% P | 5% P | 10% P |
| | 5% T | 10% T | 5% T | 10% T | 5% T | 10% T | 5% T | 10% T |

| | |
|---|---|
| Total Volume | 100 |

“P” indicates pullulan and “T” indicates trehalose

100 μL aliquots of the samples were foam dried in freeze dryer 2 mL glass vials for 21 hours using a two-stage drying protocol. The first (primary) drying stage was 10 hours at 4° C. The second stage was 4.5 hours at 25° C.. Between the first and second stage, the temperature ramped from 4-25° C. over 6.5 hours. The samples were pre-cooled to 4° C. for 30 minutes before starting the drying. The pressure setpoint during the entire 21 hours was about 16 pBar. After drying, samples were stoppered and crimped.

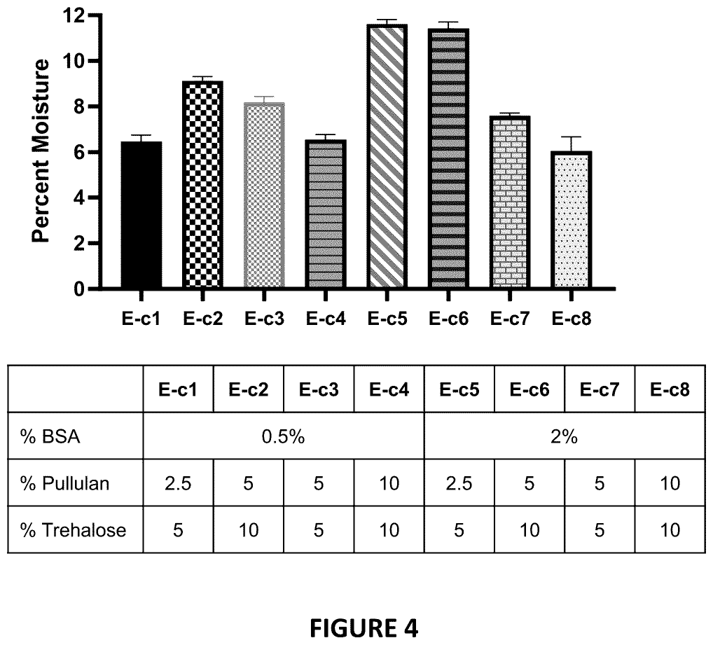

FIG. 4 presents the RM of the samples.

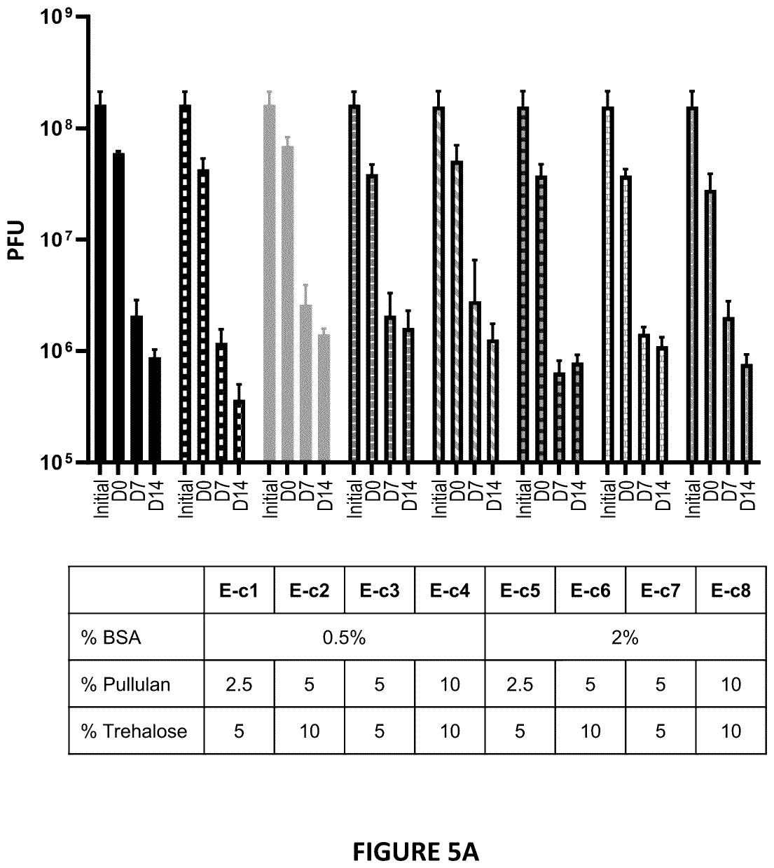

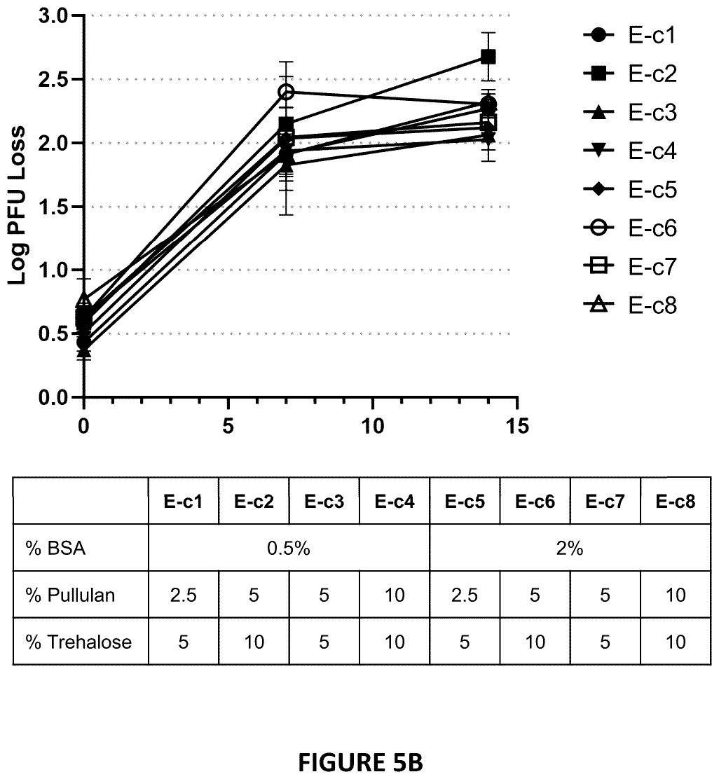

The samples were incubated at 37° C. for 7 days. FIGS. 5A-5B present the results of this study. Table 8 below shows the wt % of the components in the dry formulation.

TABLE 8

| | E-c1 | | E-c1 | E-c2 | | E-c2 | E-c3 | | E-c3 | E-c4 | | E-c4 |
|---|---|---|---|---|---|---|---|---|---|---|---|---|
| | (As made | | Wt % Dried | (As made | | Wt % Dried | (As made | | Wt % Dried | (As made | | (Wt % Dried |
| | Formulation) | | Formulation | Formulation) | | Formulation | Formulation) | | Formulation | Formulation) | | Formulation) |
| Pullulan | 2.5 | wt % | 28.5 | 5 | wt % | 30.7 | 5 | wt % | 44.3 | 10 | wt % | 47.0 |
| Trehalose | 5 | wt % | 57.0 | 10 | wt % | 61.4 | 5 | wt % | 44.3 | 10 | wt % | 47.0 |
| BSA | 0.5 | wt % | 6.3 | 0.5 | wt % | 43.4 | 0.5 | wt % | 4.9 | 0.5 | wt % | 2.6 |
| NaCl | 50 | mM | 3.7 | 50 | mM | 2.0 | 50 | mM | 2.9 | 50 | mM | 1.5 |
| Tris | 10 | mM | 1.5 | 10 | mM | 0.8 | 10 | mM | 1.2 | 10 | mM | 0.6 |
| Gelatin | 0.01 | wt % | 0.1 | 0.01 | wt % | 0.03 | 0.01 | wt % | 0.05 | 0.01 | wt % | 0.03 |
| MgSO4 | 10 | mM | 1.5 | 10 | mM | 0.8 | 10 | mM | 1.2 | 10 | mM | 0.6 |
| CaCl2 | 10 | mM | 1.4 | 10 | mM | 0.8 | 10 | mM | 1.1 | 10 | mM | 0.6 |

| | | | | | | | | |
|---|---|---|---|---|---|---|---|---|
| Virus | 1e8 particles | 0.001 | 1e8 particles | 0.0003 | 1e8 particles | 0.0004 | 1e8 particles | 0.0002 |

### Example 16: PT/CM/BSA/NaCl Composition Dried at 4° C./25° C. with Nitrogen Backfill

Serum free VSV-GFP was transferred from an initial solution to a dialysis buffer solution by way of buffer exchange using a spin column to create two stock solutions.

Two formulations were prepared using 10% by volume of stock solution and 90% by volume of a solution of either a) pullulan and trehalose or b) trehalose, each dissolved in one of the dialysis buffers, as indicated in Table 9 below.

TABLE 9
Formulations for The Study

| | E-c1 | Trehalose |
|---|---|---|
| VSV Stock | GFP (Lot 357) | |
| Dialysis Buffer (pH 7.2) | CM + 0.5% BSA + | Tris |
| | 50 mM NaCl | |
| Formulation | 2.5% Pullulan | 5% Trehalose |
| (+Dialysis Buffer) | 5% (0.125M) Trehalose | |

| | |
|---|---|
| Total Volume | 100 |

100 μL aliquots of the samples were foam dried in freeze dryer 2 mL glass vials for 19.5 hours using a two-stage drying protocol. The first (primary) drying stage was 8.5 hours at 4° C. The second stage was 6.5 hours at 25° C. Between the first and second stage, the temperature ramped from 4-25° C. over 4.5 hours. The pressure setpoint during the entire 21 hours was about 12 pBar. After drying, samples were backfilled with nitrogen gas, then stoppered and crimped.

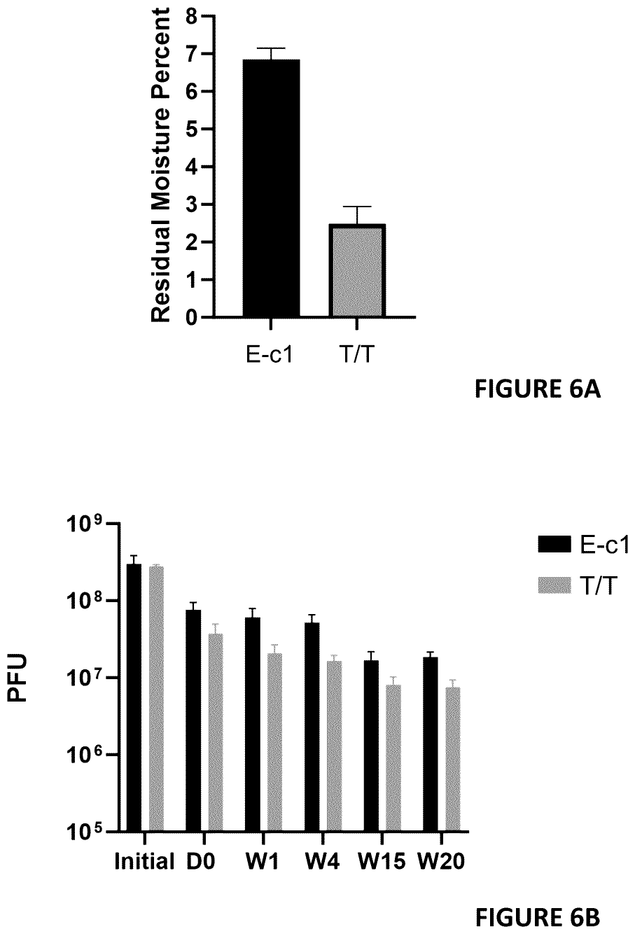

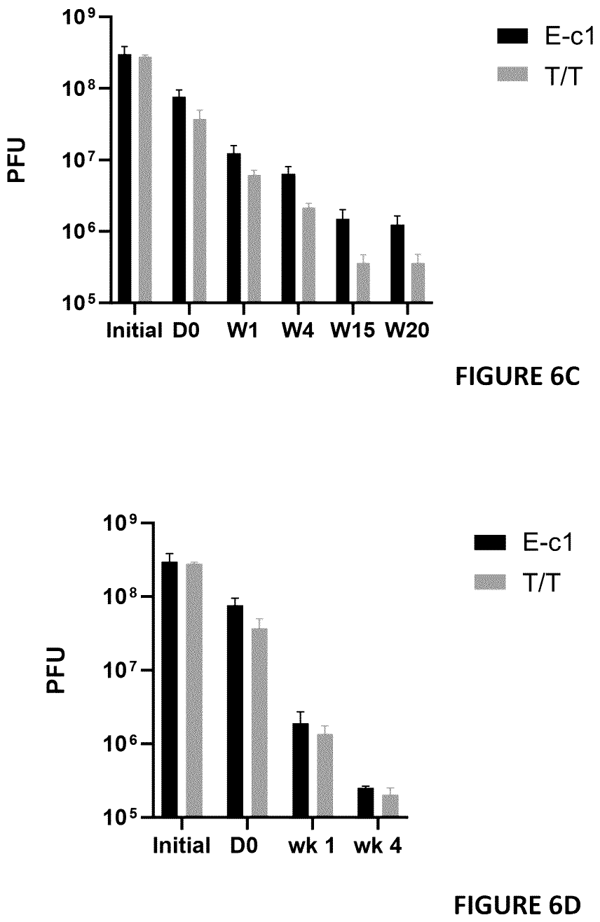

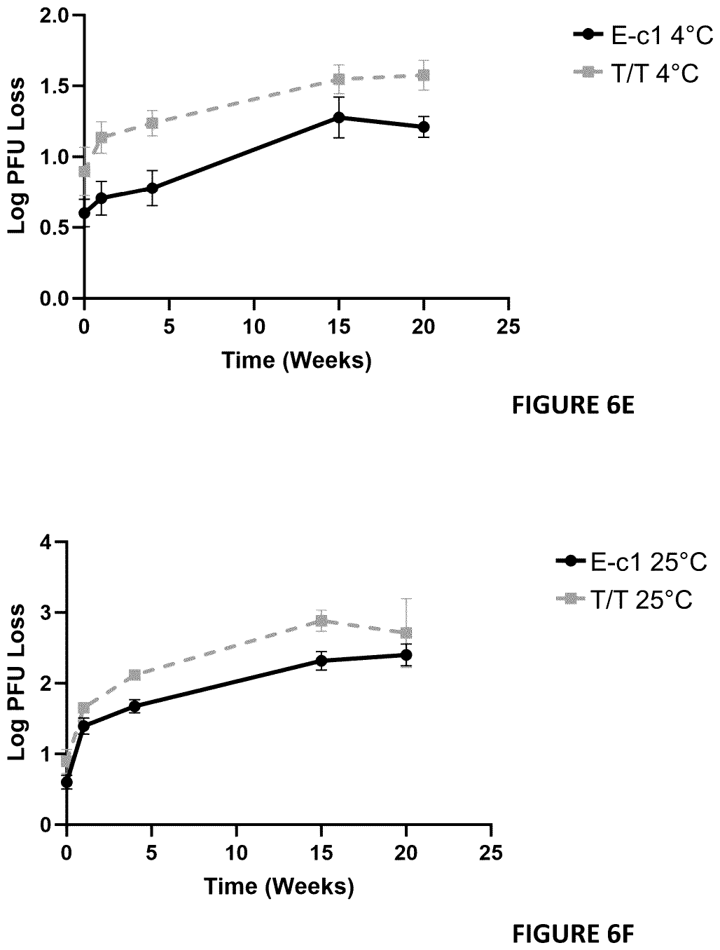

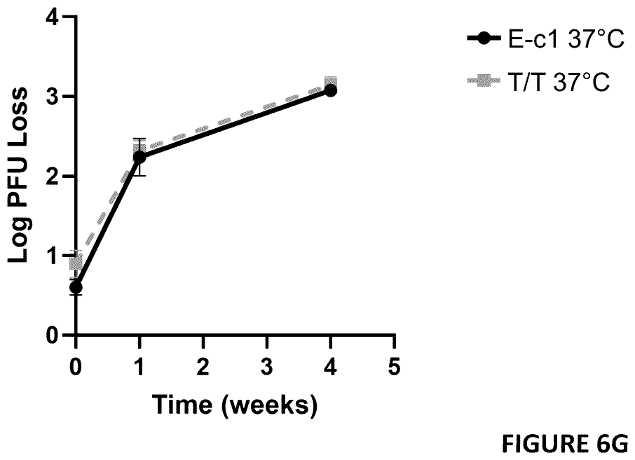

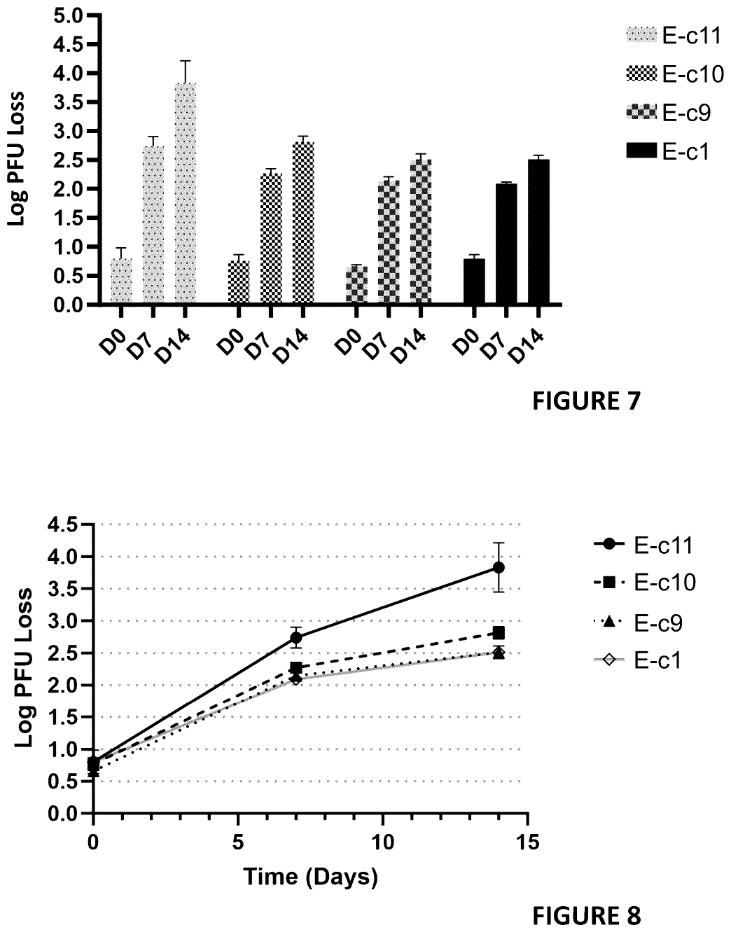

The residual moisture (RM) of the E-c1 formulation was about 6.8%. The residual moisture of the Trehalose formulation was about 2.5% (FIG. 6A). There was less than 1 log of process loss for both samples.

The samples were incubated at 4° C., 25° C. for 20 weeks and at 37° C. for 4 weeks. FIGS. 6B-6G present the results of this study.

### Example 17: PT/CM/BSA/NaCl Composition Dried at 4° C./25° C. with varying PT concentrations

Serum free VSV-GFP was transferred from an initial solution to a dialysis buffer solution by way of buffer exchange using a spin column to create two stock solutions.

Four formulations were prepared using 10% by volume of stock solution and 90% by volume of a solution of pullulan and trehalose, each dissolved in the dialysis buffers, with varying concentrations of pullulan and trehalose as indicated in Table 10 below.

TABLE 10
Formulations for The Study

| | E-c1 | E-c9 | E-c10 | E-c11 |
|---|---|---|---|---|
| VSV Stock | GFP (Lot 357) | | | |
| Dialysis Buffer (pH 7.2) | CM + 0.5% BSA + 50 mM NaCl | | | |
| Formulation (+Dialysis | 2.5% Pullulan | 1.25% Pullulan | 0.625% Pullulan | 0.25% Pullulan |
| Buffer) | 5% Trehalose | 2.5% Trehalose | 1.25% Trehalose | 0.625% Trehalose |

| | |
|---|---|
| Total Volume | 10 ul Virus + 90 ul formulation = 100 ul |

100 μL aliquots of the samples were foam dried in freeze dryer 2 mL glass vials for 23 hours using a two-stage drying protocol. The first (primary) drying stage was 10 hours at 4° C. The second stage was 6.5 hours at 25° C.. Between the first and second stage, the temperature ramped from 4-25° C. over 6.5 hours. The pressure setpoint during the entire 23 hours was about 12 uBar. After drying, the samples were stoppered inside of dryer so only inert Nitrogen was in sample vial.

The residual moisture (RM) of the formulation showed minimum PT is 0.625% P, 1.25% T and RM reduced with higher PT concentrations (FIG. 8A). FIG. 8B shows the dry weight of the compositions.

The samples were incubated at 37° C. for two weeks. FIGS. 8C-8D present the results of this study. Process loss for all formulations was about 0.9 log. After 14 days, titer loss of about 2.5 log for 5% and 2.5% Trehalose formulations. Table 11 below shows the wt % of the components in the dry formulation.

TABLE 11

| | E-c1 | | E-c1 | E-c9 | | E-c9 | E-c10 | | E-c10 | E-c11 | | E-c11 |
|---|---|---|---|---|---|---|---|---|---|---|---|---|
| | (As made | | wt % Dried | (As made | | wt % Dried | (As made | | wt % Dried | (As made | | (Wt % Dried |
| | Formulation) | | Formulation | Formulation) | | Formulation | Formulation) | | Formulation | Formulation) | | Formulation) |
| Pullulan | 2.5 | wt % | 28.5 | 1.25 | wt % | 24.9 | 0.625 | wt % | 19.8 | 0.25 | wt % | 11.5 |
| Trehalose | 5 | wt % | 57.0 | 2.5 | wt % | 49.7 | 1.25 | wt % | 39.7 | 0.625 | wt % | 29.0 |
| BSA | 0.5 | wt % | 6.3 | 0.5 | wt % | 11.1 | 0.5 | wt % | 17.6 | 0.5 | wt % | 25.8 |
| NaCl | 50 | mM | 3.7 | 50 | mM | 6.5 | 50 | mM | 10.3 | 50 | mM | 15.1 |
| Tris | 10 | mM | 1.5 | 10 | mM | 2.7 | 10 | mM | 4.3 | 10 | mM | 6.3 |
| Gelatin | 0.01 | wt % | 0.1 | 0.01 | wt % | 0.11 | 0.01 | wt % | 0.18 | 0.01 | wt % | 0.26 |
| MgSO4 | 10 | mM | 1.5 | 10 | mM | 2.7 | 10 | mM | 4.2 | 10 | mM | 6.2 |
| CaCl2 | 10 | mM | 1.4 | 10 | mM | 2.5 | 10 | mM | 3.9 | 10 | mM | 5.7 |

| | | | | | | | | |
|---|---|---|---|---|---|---|---|---|
| Virus | 1e8 particles | 0.001 | 1e8 particles | 0.0009 | 1e8 particles | 0.0014 | 1e8 particles | 0.0021 |

### Example 18: PT/CM/BSA/NaCl Dialyzed and non-Dialyzed Composition with and without Sucrose Dried at 15° C./25° C.

FBS free VSV-GFP was transferred from an initial solution to a dialysis buffer solution by way of buffer exchange using a Zebra spin column (0.5 mL columns) to create two stock solutions.

Four formulations were prepared using 10% by volume of stock solution and 90% by volume of a formulation solution, each dissolved in one of the dialysis buffers, as indicated in Table 12 below.

TABLE 12
Formulations for The Study

| | E-c1 | Sample 1 | Sample 2 | Sample 3 | Sample 4 |
|---|---|---|---|---|---|
| VSV Stock | GFP | Dialyzed | Non-Dialyzed | Dialyzed | Dialyzed |
| | (Lot 357) | 10 ul | 10 ul | 10 ul | 10 ul |
| Dialysis Buffer | CM + 0.5% | CM + 0.5% | [symbol] | CM + 0.5% | CM + 0.5% |
| (pH 7.2) | BSA + 50 mM | BSA + 50 mM | | BSA + 50 mM | BSA + 50 mM |
| | NaCl | NaCl | | NaCl | NaCl + 4% |
| | | | | | sucrose |
| Formulation | 2.5% Pullulan | 90 ul | 90 ul | 90 ul formulation | 90 ul |
| (+Dialysis Buffer) | 5% Trehalose | | | buffer + 490 ul | |
| | | | | dialysis buffer | |
| Total Volume | 100 | 100 ul | 100 ul | 590 ul | 100 ul |

The samples were dried in freeze dryer in 2 mL glass vials for 23 hours using a two-stage drying protocol. The first (primary) drying stage was 10 hours at 150° C. The second stage was 6.5 hours at 25° C.. Between the first and second stage, the temperature ramped from 15-25° C. over 6.5 hours. The samples were pre-cooled at 150° C. for 30 minutes before starting the drying. The pressure setpoint during the entire 21 hours was about 12 pBar. After drying, samples were backfilled with nitrogen gas, then stoppered and crimped.

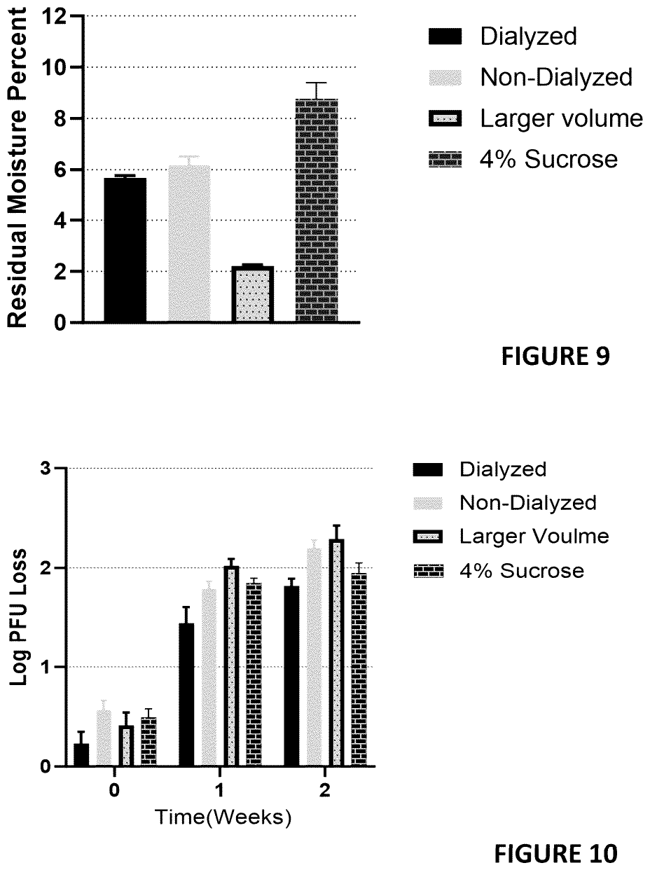

FIG. 9 presents the RM after drying of the samples.

The samples were incubated at 37° C. for 14 days. FIG. 10 present the results of this study.

### Example 19: PT Dried Under Ambient Pressure and Temperature

Serum free VSV was obtained in a stock solution containing 100 mM HEPES buffer, 150 mM NaCl and 4% sucrose. Aliquots were prepared by mixing 1 μL of a serum free VSV stock solution with 9 μL of a solution containing 10 wt % pullulan and 20 wt % trehalose in water in 1.7 mL centrifuge tubes. Residual moisture of the samples was not measured since the samples were too small for accurate measurement. The initial (before drying) titer of each aliquot was approximately 10⁷0.6 PFU. The aliquots were air dried in a biosafety cabinet for 3 days at ambient pressure and 25° C. After drying, the aliquots were below the detection limit (10¹ PFU) of the titer assay.

### Example 20: CM/BSA/NaCl/PT Dried Under Ambient Pressure and Temperature

Serum free VSV was transferred from a stock solution to a CM buffer. Aliquots were prepared by mixing 1 μL of the VSV/CM buffer solution into 9 μL of a solution having 0.5% BSA, 50 mM NaCl, 2.5 wt % pullulan and 5 wt % trehalose in CM buffer in 1.7 mL centrifuge tubes (E-c1). The initial (before drying) titer of each aliquot was approximately 10⁷0.6 PFU. The aliquots were air dried in a biosafety cabinet for 3 days at ambient pressure and 25° C.

Residual moisture of the samples was not measured since the samples were too small for accurate measurement. Aliquots tested immediately after drying (DO) had a titer of approximately 10^{5.8} PFU, indicating a process loss of about 1.8 logs. Aliquots tested after drying and incubation at 37° C. for 7 days (D7) had a titer of approximately 104-5 PFU, indicating a total loss of over 3 logs. Example 21 (CM/BSA/NaCl/PT 800 mBar vacuum dried)

Serum free VSV was transferred from an initial solution to a dialysis buffer solution by way of buffer exchange using a spin column. A mixture was prepared having 10% by volume of the VSV in CM buffer and 90% by volume of a solution having 0.5% BSA, 50 mM NaCl, 2.5 wt % pullulan and 5 wt % trehalose in CM buffer. 100 μL aliquots of the mixture were transferred to 2 mL glass vials. The initial titer of the samples was 10^{8.2} PFU. The aliquots were dried at 4° C. for 22 hours at a pressure of 800 mBar. At the end of the 22 hours, the samples were still liquid. The samples were then dried for a further 22 hours at 800 mBar with the temperature ramping from 4° C. to 25° C. for 9 hours and then held at 25° C. for 13 hours. After the entire 44 hours of drying, a clear film had formed in the vials and the vials were stoppered and crimped. The residual moisture was 10%. Samples tested immediately after drying had a titer of 10^{6,7} PFU indicating a process loss after drying of 1.5 log.

Aliquots of another mixture prepared as described in the paragraph above were dried at 8° C. for 45 hours at a pressure of 800 mBar. The initial titer of the samples was 10^{8.3} PFU. After the 45 hours of drying, a clear film had formed in the vials and the vials were stoppered and crimped. The residual moisture in was 8%. Samples tested immediately after drying had a titer of 10⁷ PFU indicating a process loss after drying of 1.3 log.

No foam was formed in these experiments. The process loss with both vacuum drying methods was higher than for any foam drying method tested. Heat challenge tests on the vacuum dried samples were not conducted.

### Example 22: Various Formulations Dried at 4° C./25° C.

Serum free VSV was transferred from an initial solution to a dialysis buffer solution by way of buffer exchange using a spin column to create a stock solution. The dialysis buffer contained 10 mM Tris-HCl.

Five formulations were prepared using 10% by volume of the stock solution and 90% by volume of a solution as indicated in Table 1. 100 μL aliquots of the samples were foam dried in freeze dryer in 2 mL glass vials. The drying temperature was 4° C. for 10 hours, followed by 6.5 hours ramping from 4° C. to 25° C., followed by 6.5 hours at 25° C. The cabinet pressure set point during the 23 hours drying cycle was 17 uBar. After drying, samples were backfilled with nitrogen gas, then stoppered and crimped.

TABLE 13
Formulations for The Study

| | | Tris PT | Tris PT | Tris PT | |
|---|---|---|---|---|---|
| | Tris PT | NaCl | BSA | NaCl BSA | E-c1 |
| Tris | 10 mM | 10 mM | 10 mM | 10 mM | 10 mM |
| MgSO4 | | | | | 10 mM |
| CaCl2 | | | | | 10 mM |
| Gelatin | | | | | 0.05% |
| BSA | | | 0.5% | 0.5% | 0.5% |
| NaCl | | 50 mM | | 50 mM | 50 mM |
| Pullulan | 2.5% | 2.5% | 2.5% | 2.5% | 2.5% |
| Trehalose | 5% | 5% | 5% | 20% | 5% |

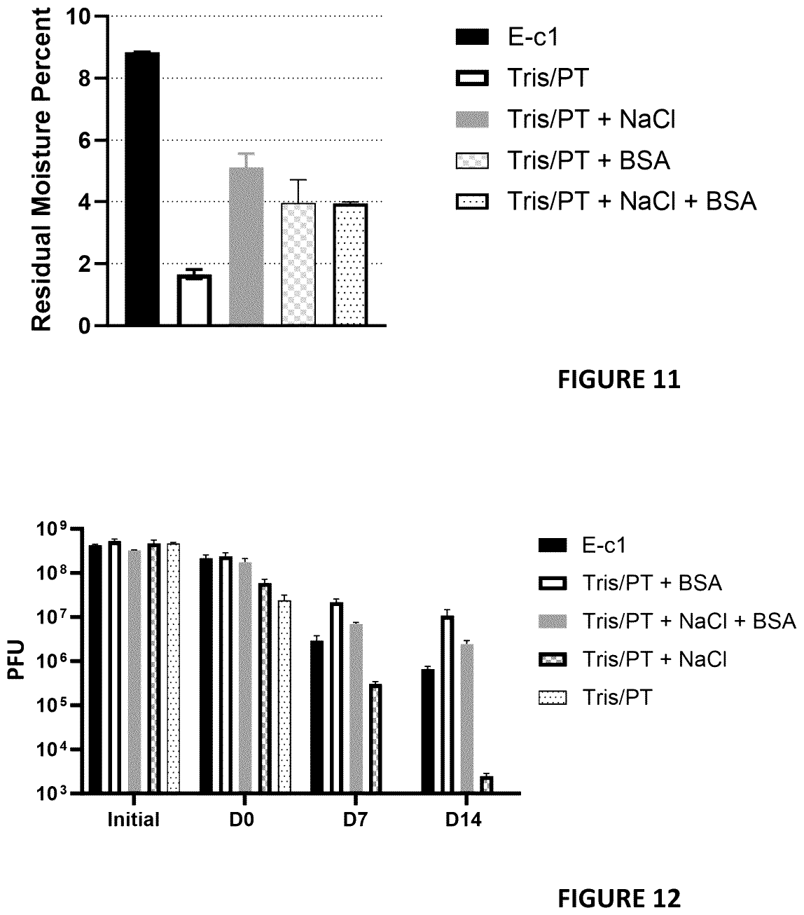

Residual moisture of the samples after drying is shown in FIG. 11.

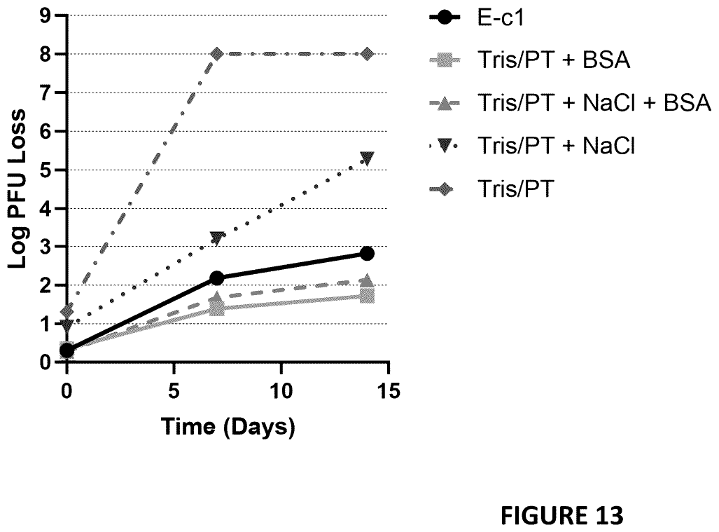

Samples were tested before drying (Initial), immediately after drying (DO), after 7 days of incubation at 37° C. (D7) and after 14 days of incubation at 37° C. (D14). Process loss was about 0.3 log for BSA containing formulations. Tris PT had a process loss of 1.3 log and PFU counts below the detection limits of the assay at D7 and D14. Other data is presented in FIGS. 12 and 13. The data for Tris PT was considered unreliable and re-tested in the example below.

TABLE 14
Wt % of Formulations

| | | | | | | Tris/PT + | | | | | | Tris/PT + | | | |
|---|---|---|---|---|---|---|---|---|---|---|---|---|---|---|---|
| | | | | | | BSA | | | | | | NaCl | | | Tris/PT |
| | | | | Tris/PT + | | Wt % | Tris/PT + | | Tris/PT + | Tris/PT + | | (Wt % | | | (Wt % |
| | E-c1 | | E-c1 | BSA | | Dried | NaCl + BSA | | NaCl + BSA | NaCl | | Dried | Tris/PT | | Dried |
| | (As made | | Wt % Dried | (As made | | Formula- | (As made | | (Wt % Dried | (As made | | Formula- | (As made | | Formula- |
| | Formulation) | | Formulation | Formulation) | | tion | Formulation) | | Formulation) | Formulation) | | tion) | Formulation) | | tion) |
| Pullulan | 2.5 | wt % | 28.5 | 2.5 | wt % | 30.5 | 2.5 | wt % | 29.4 | 2.5 | wt % | 31.4 | 2.5 | wt % | 32.7 |
| Trehalose | 5 | wt % | 57.0 | 5 | wt % | 61.0 | 5 | wt % | 58.7 | 5 | wt % | 62.8 | 5 | wt % | 65.5 |
| BSA | 0.5 | wt % | 6.3 | 0.5 | wt % | 6.8 | 0.5 | wt % | 6.5 | — | | 1 | — | | — |
| NaCl | 50 | mM | 3.7 | — | | — | 50 | mM | 3.8 | 50 | mM | 4.1 | — | | 0.0 |
| Tris | 10 | mM | 1.5 | 10 | mM | 1.6 | 10 | mM | 1.6 | 10 | mM | 1.7 | 10 | mM | 1.8 |

| | | | | | | | | | | | |
|---|---|---|---|---|---|---|---|---|---|---|---|
| Gelatin | 0.01 | wt % | 0.1 | — | — | — | — | — | — | — | — |
| MgSO4 | 10 | mM | 1.5 | — | — | — | — | — | — | — | — |
| CaCl2 | 10 | mM | 1.4 | — | — | — | — | — | — | — | — |

| | | | | | | | | | | |
|---|---|---|---|---|---|---|---|---|---|---|
| Virus | 1e8 particles | 0.001 | 1e8 particles | 0.0005 | 1e8 particles | 0.0005 | 1e8 particles | 0.0006 | 1e8 particles | 0.0006 |

### Example 23: Various Formulations Dried at 4° C./25° C.

Serum free VSV was transferred from an initial solution to a dialysis buffer solution by way of buffer exchange using a spin column to create a stock solution. The dialysis buffer contained 10 mM Tris-HCl.

Formulations were prepared using 10% by volume of the stock solution and 90% by volume of a solution as indicated in Table 1. 100 μL aliquots of the samples were foam dried in freeze dryer in 2 mL glass vials. The drying temperature was 4° C. for 10 hours, followed by 6.5 hours ramping from 4° C. to 25° C., followed by 6.5 hours at 25° C. The cabinet pressure set point during the 23 hours drying cycle was 17 uBar. After drying, samples were backfilled with nitrogen gas, then stoppered and crimped.

TABLE 15
Formulations for The Study

| | | | | | | | | | | | | Tris/ | | | |
|---|---|---|---|---|---|---|---|---|---|---|---|---|---|---|---|
| | | | A | | | Tris/PT | | | Tris/PT + | Tris/ | | Trehalose + | | | |
| | | | Wt % | | | (Wt % | Tris/PT + | | BSA Wt % | Trehalose + | | BSA (Wt % | Tris/ | | Tris/ |
| | A | | Dried | Tris/PT | | Dried | BSA | | Dried | BSA | | Dried | Trehalose | | Trehalose |
| | (As made | | Formula- | (As made | | Formula- | (As made | | Formula- | (As made | | Formula- | (As made | | (Wt % Dried |
| | Formulation) | | tion | Formulation) | | tion) | Formulation) | | tion | Formulation) | | tion) | Formulation) | | Formulation) |
| Pullulan | 2.5 | wt % | 28.5 | 2.5 | wt % | 32.7 | 2.5 | wt % | 30.5 | — | | — | — | | — |
| Trehalose | 5 | wt % | 57.0 | 5 | wt % | 65.5 | 5 | wt % | 61.0 | 5 | wt % | 87.9 | 5 | wt % | 97.4 |
| BSA | 0.5 | wt % | 6.3 | — | | — | 0.5 | wt % | 6.8 | 0.5 | wt % | 9.8 | — | | — |
| NaCl | 50 | mM | 3.7 | — | | — | — | | — | | | — | — | | — |
| Tris | 10 | mM | 1.5 | 10 | mM | 1.8 | 10 | mM | 1.6 | 10 | mM | 2.4 | 10 | mM | 2.6 |

| Gelatin | 0.01 | wt % | 0.1 | — | — | — | — | — | — | — | — |
|---|---|---|---|---|---|---|---|---|---|---|---|
| MgSO4 | 10 | mM | 1.5 | — | — | — | — | — | — | — | — |
| CaCl2 | 10 | mM | 1.4 | — | | | | | | — | — |

| | | | | | | | | | | |
|---|---|---|---|---|---|---|---|---|---|---|
| Virus | 1e8 particles | 0.001 | 1e8 particles | 0.0006 | 1e8 particles | 0.0005 | 1e8 particles | 0.001 | 1e8 particles | 0.001 |

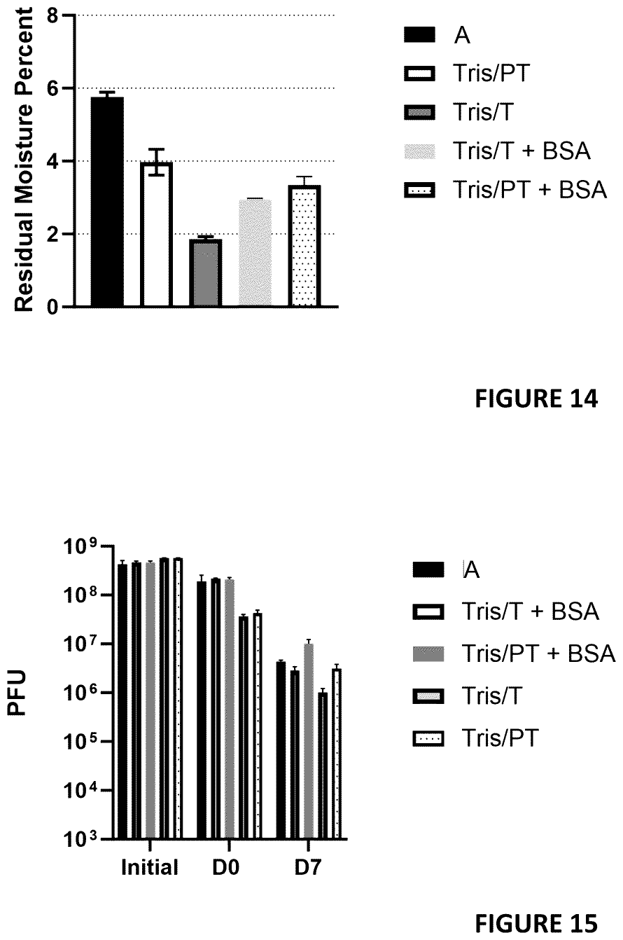

Residual moisture of the samples after drying is shown in FIG. 14.

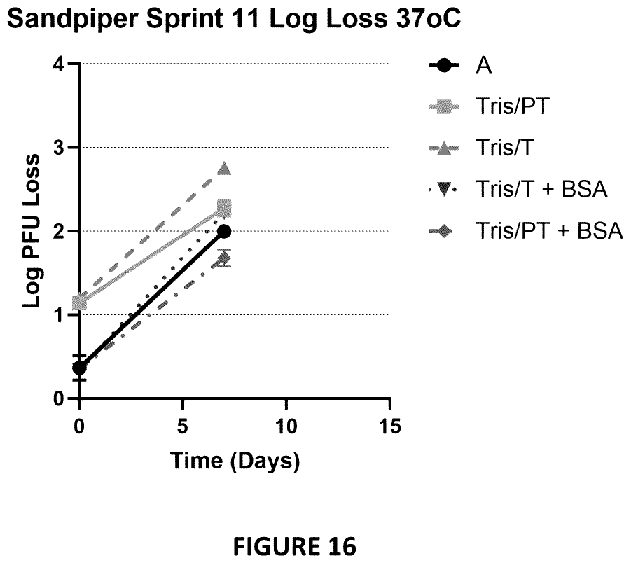

Samples were tested before drying (Initial), immediately after drying (DO), after 7 days of incubation at 37° C. (D7) and after 14 days of incubation at 37° C. (D14). Data is presented in FIGS. 15 and 16.

### Example 24

AdV-GFP was transferred from its stock buffer into a sample buffer by buffer exchange using 0.5 mL Zeba columns. 10 parts by volume of the sample buffer was mixed with 90 parts by volume of a solution of additional excipients dissolved in an additional amount of the sample buffer.

In Formulation A, the sample buffer has 10 mM Tris-HCl (pH 7.2), 10 mM MgSO₄, 10 mM CaCl₂, 0.005% Gelatin, 0.5% BSA and 50 mM NaCl. The additional excipients are 2.5% Pullulan and 5% Trehalose. Formulation A on a fully dried basis has the following components: Pullulan 28.5 wt %; Trehalose 57.0 wt %; BSA 6.3 wt %; NaCl 3.7 wt %, Tris 1.5 wt %; Gelatin 0.1 wt %; MgSO₄ 1.5 wt %; CaCl₂ 1.4 wt %; and, virus 0.001 wt %.

In Formulation B (Inulin/Mannitol), the sample buffer has 10 mM Tris-HCl (pH 8.2), 1 mM MgSO₄ and 100 mM NaCl. The additional excipients are 5% Inulin and 5% Mannitol. Formulation B represents a formulation described in Berg et al. 2021 for freeze drying Chimp AdV. This publication reports an infectivity loss of about 2 log after 30 days of storage at 45° C., and an infectivity loss of about 1.5 log after 60 days of storage at 30° C.

The samples were foam dried. 100 uL samples of each formulation were placed in vials and pre-cooled to 4° C. The pre-cooled vials were transferred to a lab scale freeze dryer operated as foam dryer. The pressure setpoint was 16uBar pressure. The temperature schedule was 10 hours at 4° C., 6.5 hours of a gradient from 4° C. to 25° C., and 6.5 hours at 25° C.

After foam drying, the residual moisture (RM) of Formulation A was 6.0%. The RM of Formulation B was 2.3%.

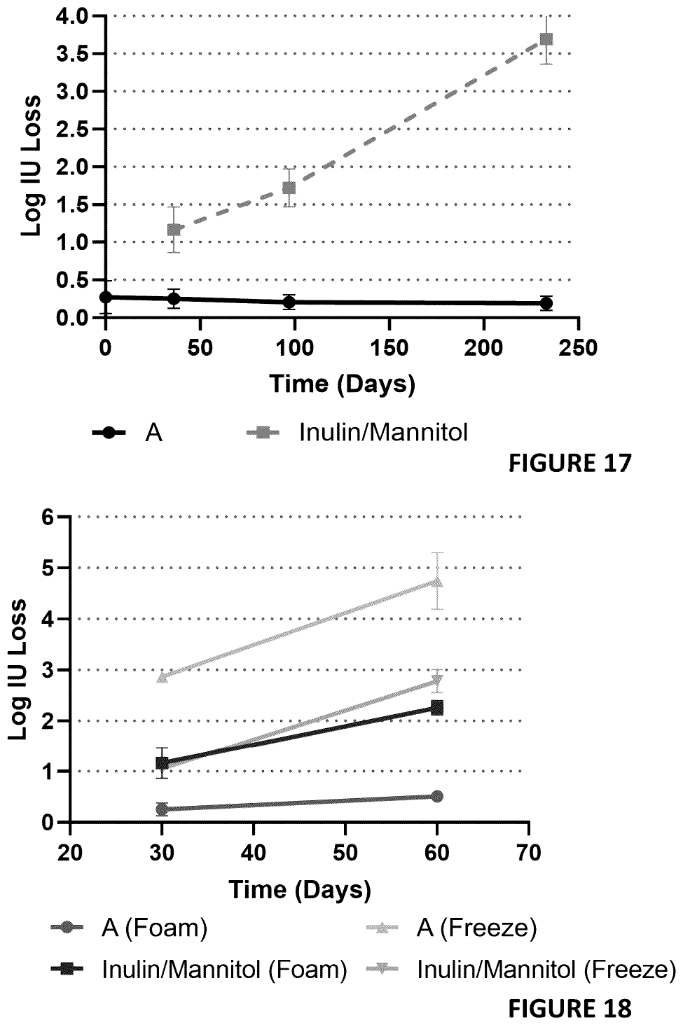

Before drying, each sample had about 10⁸ infectious units (IU). The dried samples were stored at 37° C. and tested for IU using an in-cell Western (ICW) assay with HEK293 cells. IU loss relative to the initial (before buffer exchange) IU of each formulation after drying (DO) and after storage for various time periods is shown in FIG. 17.

### Example 25

Addition samples were prepared according to Formulations A and B and foam dried as described above.

Another group of samples according to Formulations A and B were freeze dried over a 63 hour drying cycle. The primary stages of the cycle were: freezing under atmospheric pressure at −50° C.; primary drying at −50° C. (all temperatures are shelf temperature) and 0.03 mbar; secondary drying at 20° C. and 0.03 mbar; and, tertiary drying at 20° C. and 750 mbar. After After freeze drying, the residual moisture (RM) of Formulation A was 0.8%. The RM of Formulation B was 0.4%.

Before drying, each sample had about 10⁸ infectious units (IU). The dried samples were stored at 37° C. and tested for IU using an in-cell Western (ICW) assay with HEK293 cells. IU loss measured after storage relative to the initial IU of each formulation is shown in FIG. 18. Example 26

Additional samples of Formulation A and Formulation B were prepared. A third formulation, Formulation C, was also prepared as described above. In Formulation C (Tris/5% Trehalose), the sample buffer has 10 mM Tris-HCl (pH7.2). The additional excipients were 5% Trehalose.

100 uL samples of each formulation were placed in vials and foam dried. The samples were pre-cooled to 4° C. The pre-cooled vials were transferred to a lab scale freeze dryer operated as foam dryer. The pressure setpoint was 16uBar pressure. The temperature schedule was 10 hours at 4° C., 6.5 hours of a gradient from 4° C. to 25° C., and 6.5 hours at 25° C. The dryer was backfilled with nitrogen gas and the vials were stoppered inside the dryer, and crimpled immediately after being removed from the dryer.

The residual moisture (RM) of Formulation A was 7.0%. The RM of Formulation B was 1.9%. The RM of formulation C was 1.1%.

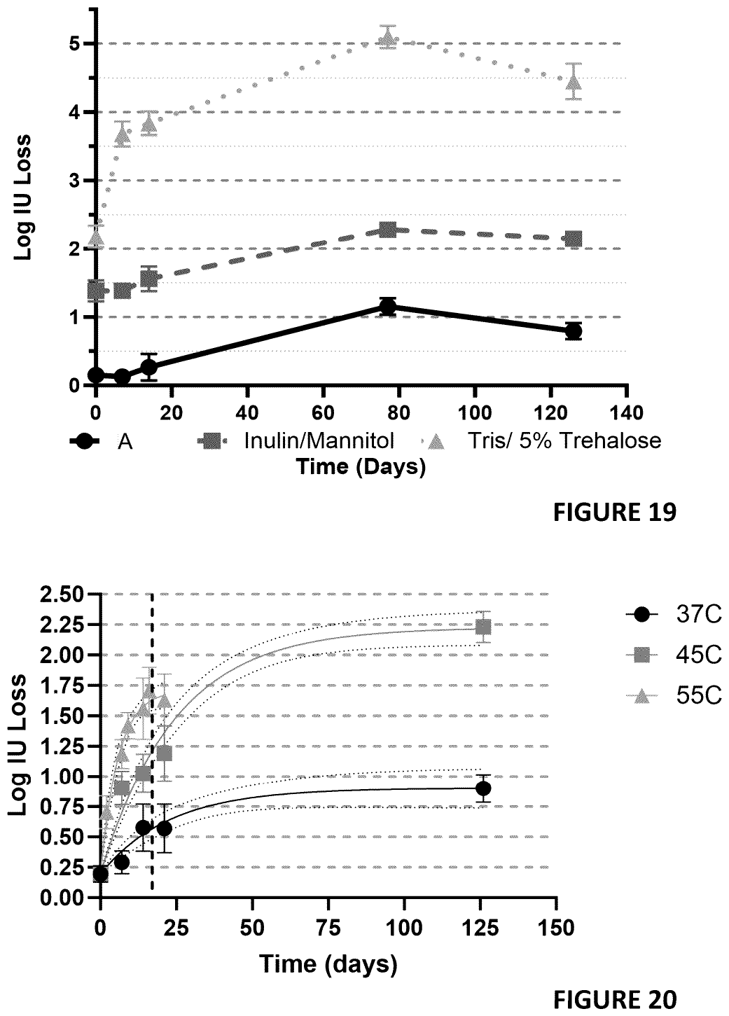

Before drying, each sample had about 10⁸ infectious units (IU). The dried samples were stored at 37° C. and tested for IU using an in-cell Western (ICW) assay with HEK293 cells. IU loss relative to the initial (before buffer exchange) IU of each formulation after drying (DO) and after storage for various time periods is shown in FIG. 19.

### Example 27

Additional samples of Formulation A were prepared.

100 uL samples of each formulation were placed in vials and foam dried. The samples were pre-cooled to 4° C. The pre-cooled vials were transferred to a lab scale freeze dryer operated as foam dryer. The pressure setpoint, initiated immediately after the pre-cooling period, was 11uBar pressure. The temperature schedule was 10 hours at 4° C., 6.5 hours of a gradient from 4° C. to 25° C., and 6.5 hours at 25° C. The dryer was backfilled with nitrogen gas and the vials were stoppered inside the dryer, and crimpled immediately after being removed from the dryer.

Unless stated otherwise, temperatures described herein are temperature setpoints which are controlled based on the temperature of the shelf inlet of the dryer. Probes placed on the wall of vials indicate that the temperature of the vials drops to below −10° C. for several minutes after the pressure setpoint is implemented and remains below 0° C. for roughly 50-100 minutes.

The residual moisture (RM) of Formulation A was 6.8%.

Before drying, each sample had about 10⁸ infectious units (IU). The dried samples were stored at 37° C., 45° C. or 55° C. and tested for IU using an in-cell Western (ICW) assay with HEK293 cells. IU loss relative to the initial (before buffer exchange) IU of Formulation A after drying (DO) and after storage for various time periods is shown in FIG. 20.

### Example 28

Additional samples of Formulation A were prepared. A fourth formulation, Formulation D (Tris PT), was also prepared as described above. In Formulation D, the sample buffer has 10 mM Tris-HCl (pH7.2). The additional excipients were 2.5% pullulan and 5% Trehalose. A fifth formulation, Formulation E, was also prepared as described above. In Formulation E, the sample buffer has 10 mM Tris-HCl (pH 7.2), 10 mM MgSO₄, 10 mM CaCl₂), 0.005% Gelatin, 0.5% BSA and 50 mM NaCl. The additional excipients are 2.5% Pullulan and 5% Trehalose and 2% Sorbitol. [00259]100 uL samples of each formulation were placed in vials and foam dried. The samples were pre-cooled to 4° C. The pre-cooled vials were transferred to a lab scale freeze dryer operated as foam dryer. The pressure setpoint was 12uBar pressure. The temperature schedule was 10 hours at 4° C., 6.5 hours of a gradient from 4° C. to 25° C., and 6.5 hours at 25° C. The dryer was backfilled with nitrogen gas and the vials were stoppered inside the dryer, and crimpled immediately after being removed from the dryer.

The residual moisture (RM) of Formulation A was 6.8%. The RM of Formulation D was 4.4%. The RM of formulation E was 12.2%.

Before drying, each sample had about 10⁸ infectious units (IU). The dried samples were stored at 55° C. and tested for IU using an in-cell Western (ICW) assay with HEK293 cells. IU loss relative to the initial (before buffer exchange) IU of each formulation after after storage for 14 days was, Formulation A: 1.8 log loss; Formulation D: 1.2 log loss; and, Formulation E: 1.6 log loss.

### Example 29

Additional samples of Formulation A and D were prepared.

100 uL samples of each formulation were placed in vials and foam dried. The vials were transferred to a lab scale freeze dryer operated as foam dryer. The pressure setpoint was 15uBar pressure. The temperature schedule was 23 hours at 25° C. The dryer was backfilled with nitrogen gas and the vials were stoppered inside the dryer, and crimpled immediately after being removed from the dryer. Despite the higher setpoint temperature, the temperature as indicated by probes attached the vials still dropped below 0° C. for a period of time.

The residual moisture (RM) of Formulation A was 5%. The RM of Formulation D was 4%.

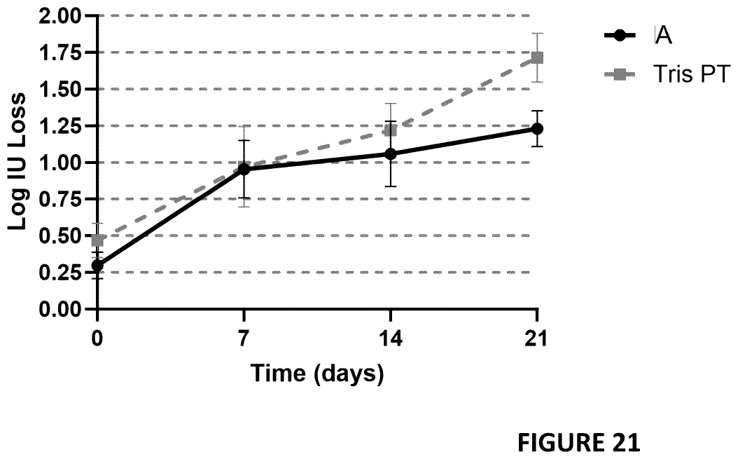

Before drying, each sample had about 10⁸ infectious units (IU). The dried samples were stored at 55° C. and tested for IU using an in-cell Western (ICW) assay with HEK293 cells. IU loss relative to the initial (before buffer exchange) IU of each formulation after storage is shown in FIG. 21.

### Example 30

Additional samples of Formulation A were prepared. A sixth formulation, Formulation F, was also prepared as described above. In Formulation F, the sample buffer has 10 mM Tris-HCl (pH7.2). The additional excipients were 2.5% pullulan, 5% Trehalose and 0.5% BSA.

100 uL samples of each formulation were placed in vials and foam dried. The vials were transferred to a lab scale freeze dryer operated as foam dryer. The pressure setpoint was 15uBar pressure. The temperature schedule was 23 hours at 25° C. The dryer was backfilled with nitrogen gas and the vials were stoppered inside the dryer, and crimpled immediately after being removed from the dryer. Despite the higher setpoint temperature, the temperature as indicated by probes attached the vials still dropped below 0° C. for a period of time.

The residual moisture (RM) of Formulation A was about 4.6%. The RM of Formulation F was about 3.3%.

Before drying, each sample had about 10⁸ infectious units (IU). The dried samples were stored at 55° C. and tested for IU using an in-cell Western (ICW) assay with HEK293 cells. IU loss relative to the initial (before buffer exchange) IU of each formulation after storage for 7 days was about 0.8 log for formulation A and about 0.9 log for Formulation F.

### Example 31

Serum free VSV was transferred from an initial solution to a dialysis buffer solution by way of buffer exchange using a spin column to create a stock solution. The dialysis buffer contained 10 mM Tris-HCl and 0.5% BSA.

Formulations were prepared using 10% by volume of the stock solution and 90% by volume of 2 formulations. Both formulations contained 10 mM Tris-HCl, 0.5% BSA and 2.5% Pullulan. One of the formulations contained 5% Trehalose and the other contained 15% Trehalose.

100 μL aliquots of the samples were vacuum dried in freeze dryer in 2 mL glass vials. The drying temperature was 8° C. for 2 hours under low vacuum, followed by 8° C. for 5 hours under medium vacuum, followed by 6.5 hours ramping from 8° C. to 25° C., followed by 6.5 hours at 25° C. The cabinet pressure set point during the first 2 hours of drying cycle was 20mBar and for the remaining 18 hours of the drying cycle, it was set to 0.01mBar. After drying, samples were backfilled with nitrogen gas, then stoppered and crimped.

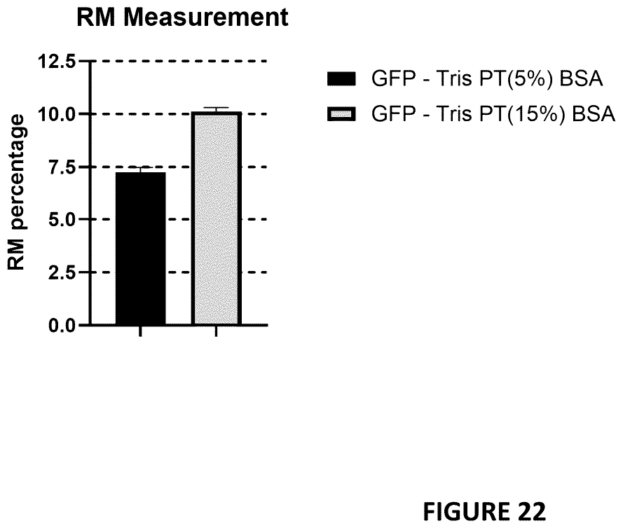

Residual moisture (RM) of the samples after drying is shown in FIG. 22. % RM was higher compared to the 4/25 high vacuum drying. Formulation with 15% Trehalose consistently had a higher % RM.

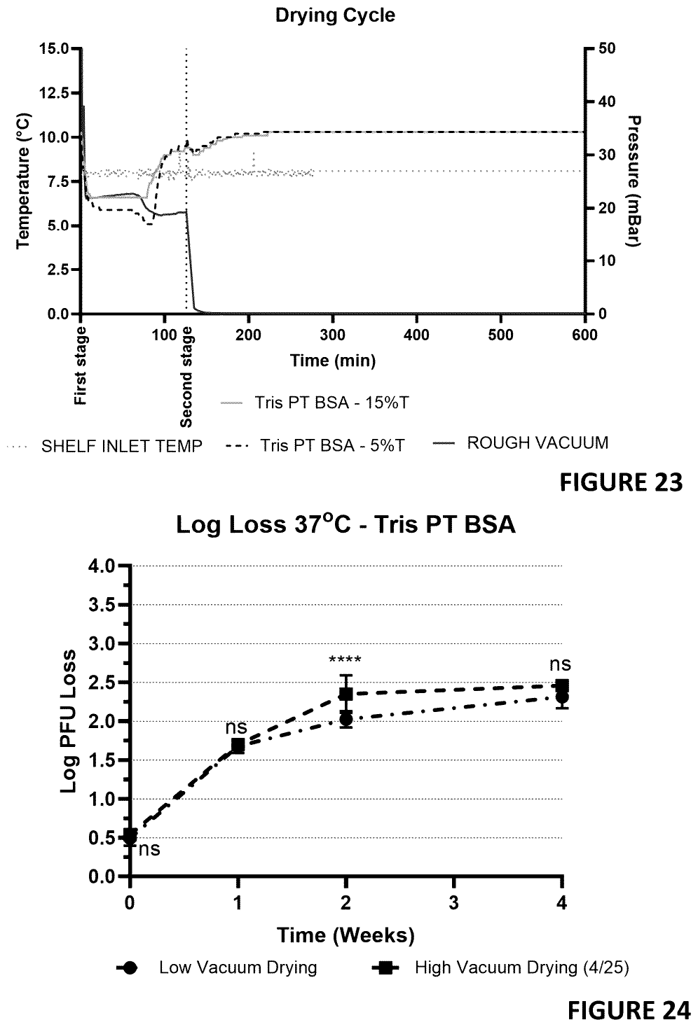

The product temperature of each formulation was tracked using thermocouples (FIG. 23). Freezing point at 8° C. and 20mBar is 0° C. First stage: shelf temperature is set to 8° C. and the pressure is decreased to 20 mBar. Pressure initially stabilized at 23 mBar but eventually decreased to 19mBar. Product temperature went below the shelf temperature by about 2-2.5 degrees. Product temperature comes back up after about 100 mins.

Second stage: shelf temperature is kept at 8° C. but the pressure is set to as low as possible to 0.01 mBar. With the new set up, the pressure stabilizes at 0.13 mBar. Product temperature stays above the shelf temperature.

Software disconnected from the system after about 600 minutes.

In the next step, two drying schedules (high vacuum and low vacuum dryings) were tested.

High Vacuum Drying: First stage: 10 hours at 4° C. and constant pressure of 12 uBar, 6.5 hrs gradient to 25° C. Second Stage: 6.5 hrs at 25° C. and constant pressure of 12 uBar.

Low Vacuum Drying: First stage: 2 hrs at 80° C. and 20mBar. Second stage: 5 hours at 8° C., 6.5 hrs gradient to 25° C. and constant pressure at 125 uBar. Third stage: 6.5 hrs at 25° C. and constant pressure of 125 uBar.

No significant difference between the two drying schedules was observed (FIG. 24).

Further, vero cells were infected with post dialysis (PD), week zero, i.e. directly after drying (WO), week 1 (W1), week 2 (W2) or week 4 (W4) samples 45 mins prior to immobilization with an overlay; plaques counted next day.

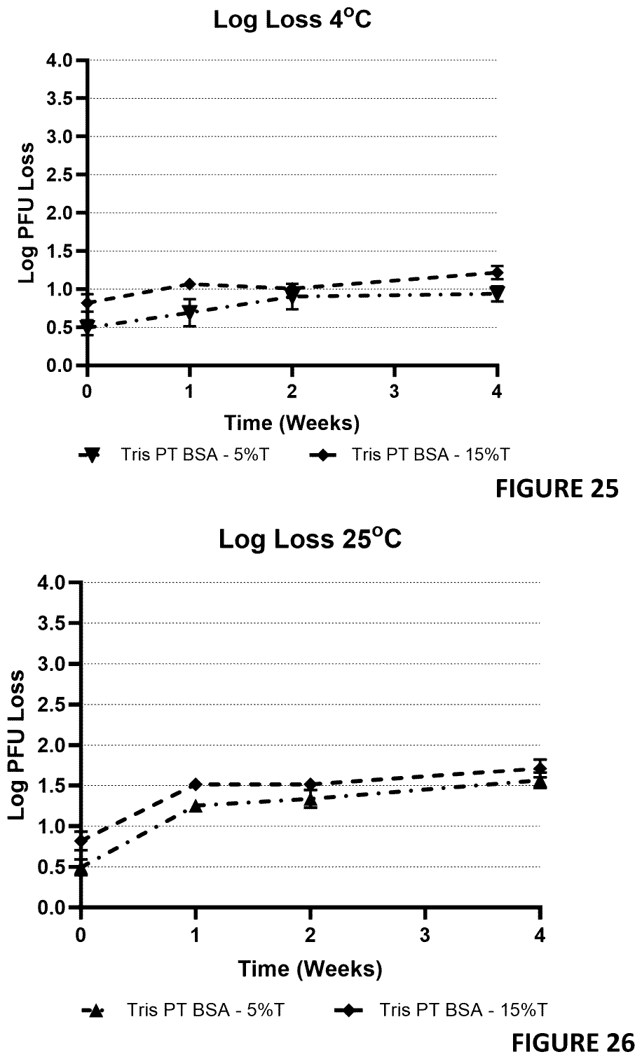

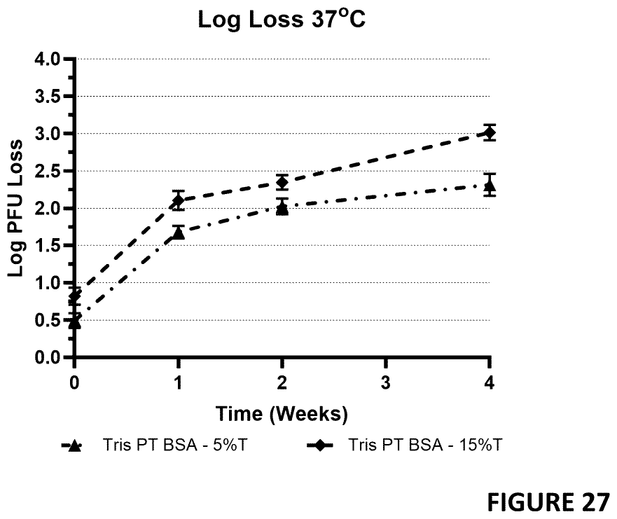

Process loss was about 0.5 log for the 5% Trehalose and 0.8 log for the 15% Trehalose. New drying process did not significantly improve VSV thermostability at all storage temperatures. Tris PT BSA with 5% Trehalose showed slightly better results than 15% Trehalose (FIGS. 25-27).

### Example 32

Serum free VSV was transferred from an initial solution to a dialysis buffer solution by way of buffer exchange using a spin column to create a stock solution. The dialysis buffer contained 10 mM Tris-HCl and 0.5% BSA.

Formulations were prepared using 10% by volume of the stock solution and 90% by volume of solutions as indicated in Table 15.

TABLE 16
Formulations for The Study

| | | Tris CMC | Tris Dex | | |
|---|---|---|---|---|---|
| | Tris PT | T BSA | T BSA | Tris P | Tris T |
| | BSA (1) | (2) | (3) | BSA (4) | BSA (5) |
| | Concentration | | | | |
| Tris | 10 mM (pH 7.2) | | | | |
| BSA | 0.5% | | | | |
| Pullulan | 2.5% | | | 2.5% | |
| CMC | | 2.5% | | | |
| Dextran | | | 2.5% | | |
| Trehalose | 5% | 5% | 5% | | 5% |

100 μL aliquots of the samples were foam dried in freeze dryer in 2 mL glass vials. The drying temperature was 4° C. for 10 hours, followed by 6.5 hours ramping from 4° C. to 25° C., followed by 6.5 hours at 25° C. The cabinet pressure set point during the 23 hours drying cycle was 10uBar. The final chamber pressure was 99uBar. After drying, samples were backfilled with nitrogen gas, then stoppered and crimped.

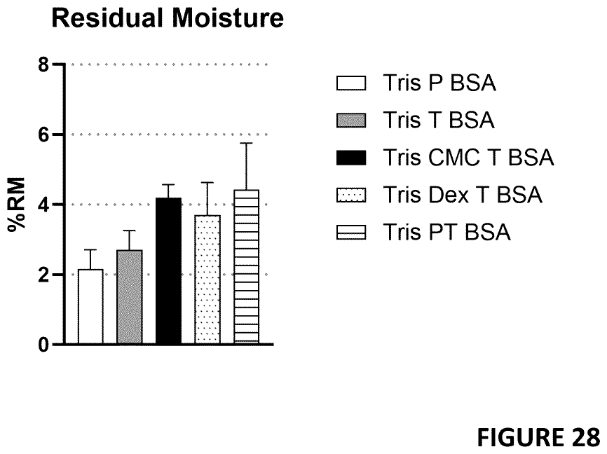

Residual moisture (RM) of the samples after drying is shown in FIG. 28. Formulations with dextran (Tris Dex T BSA) and CMC (Tris CMC T BSA) showed similar % RM to pullulan/trehalose formulation (Tris PT BSA).

Further, Vero cells were infected with PD, WO, and W1 samples 45 mins prior to immobilization with an overlay; plaques counted next day.

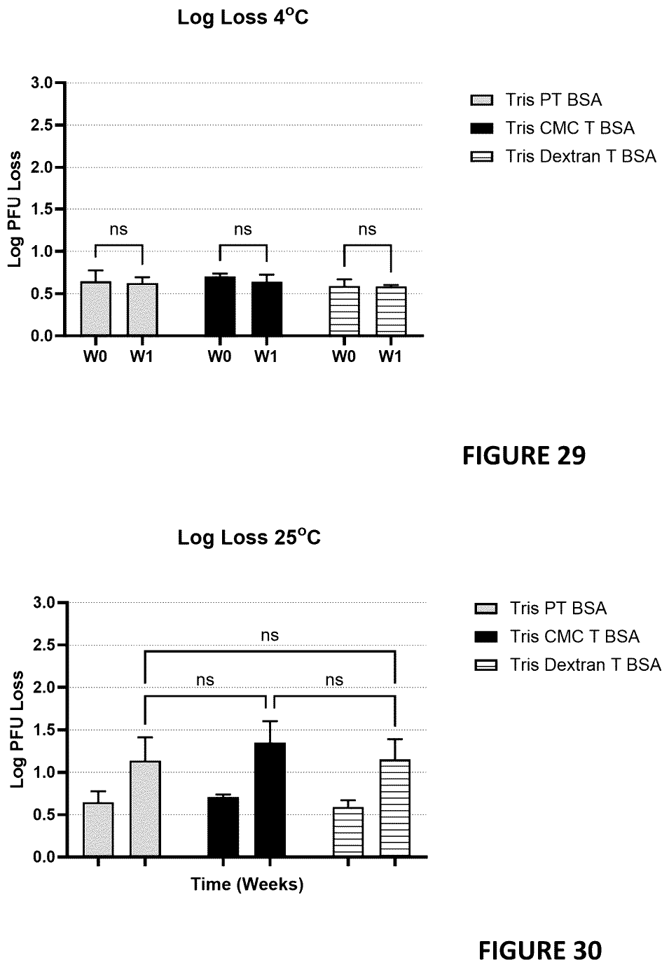

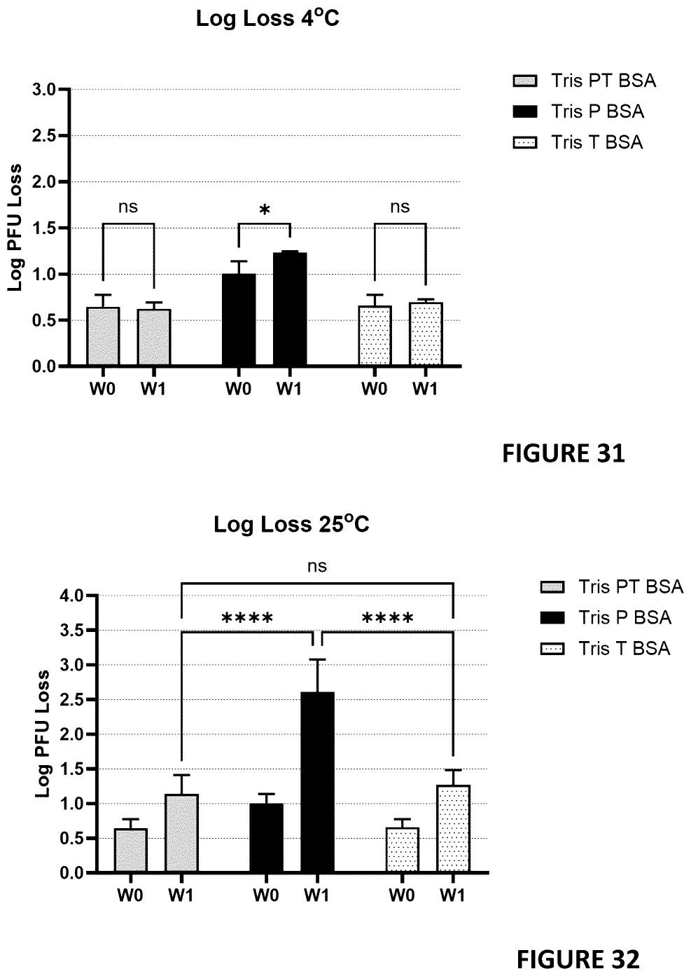

Process loss was about 0.6 log for all formulations. After 1 week incubation at 25° C., all formulations lost about 1.2 logs (FIGS. 29-32).

From these results it can be seen that CMC and Dextran 40k have the same impact on VSV stability as pullulan. Initial timepoints at 25° C.: Pullulan doesn't seem to be important for VSV stability in a 1 week trial.

### Example 33

AdV-GFP was transferred from its stock buffer into a sample buffer by buffer exchange using 0.5 mL Zeba columns. 10 parts by volume of the sample buffer was mixed with 90 parts by volume of a solution of additional excipients dissolved in an additional amount of the sample buffer, as shown in Table 17 below.

TABLE 17
Formulations for The Study

| | CM PT BSA NaCl | Berg 2021 |
|---|---|---|
| | Concentration | Concentration |
| Tris | 10 mM | 10 mM |
| MgS0₄ | 10 mM | 1 mM |
| CaCl₂ | 10 mM | |
| Gelatin | 0.005% | |
| BSA | 0.5% | |
| NaCl | 50 mM | 100 mM |
| Pullulan | 2.5% | |
| Trehalose | 5% | |
| Inulin | | 5% |
| Mannitol | | 5% |

| | |
|---|---|
| Volume | 100 uL |

The samples were foam dried or freeze dried. For foam drying, 100 uL samples of each formulation were placed in vials and pre-cooled to 4° C. The pre-cooled vials were transferred to a lab scale freeze dryer operated as foam dryer. The pressure setpoint was 16uBar pressure. The temperature schedule was 10 hours at 4° C., 6.5 hours of a gradient from 4° C. to 25° C., and 6.5 hours at 25° C. For freeze drying, 100 ul samples of each formulation were placed in vials and pre-cooled to −55° C. for 5 hours and 55 mins. Vacuum was applied to a pressure of 0.03 mBar for 5 hours and 30 mins, followed with a temperature ramp to 0° C. over 50 mins. Samples were dried at 0° C. and 0.03 mBar for 4 hours. The final stage of drying was at 20° C. for 45 hours.

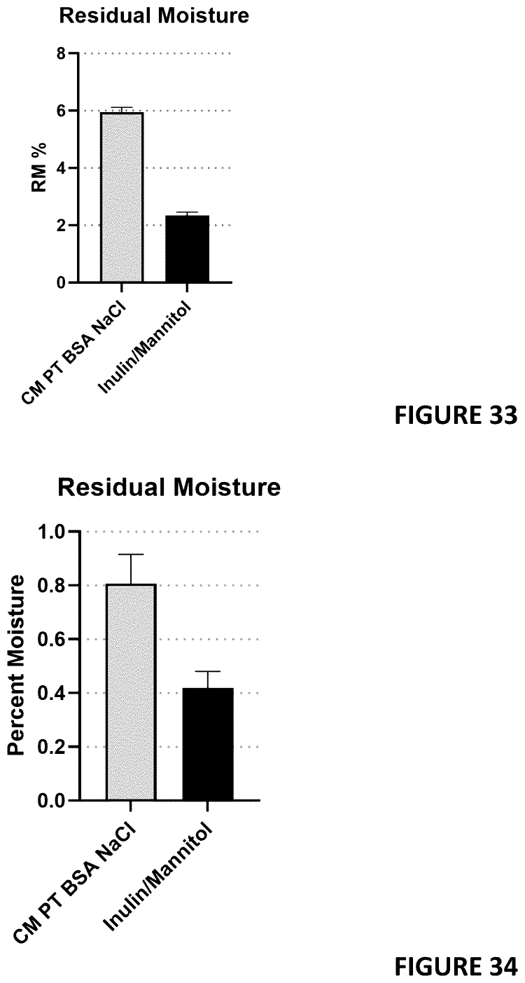

After foam drying, the residual moisture (RM) of Formulation CM PT BSA NaCl was 6.0% and the RM of Formulation Inulin/Mannitol was 2.3%. Afterfreeze drying, the RM of Formulation CM PT BSA NaCl was 0.8% and the RM of Formulation Inulin/Mannitol was 0.4% (FIGS. 33-34).

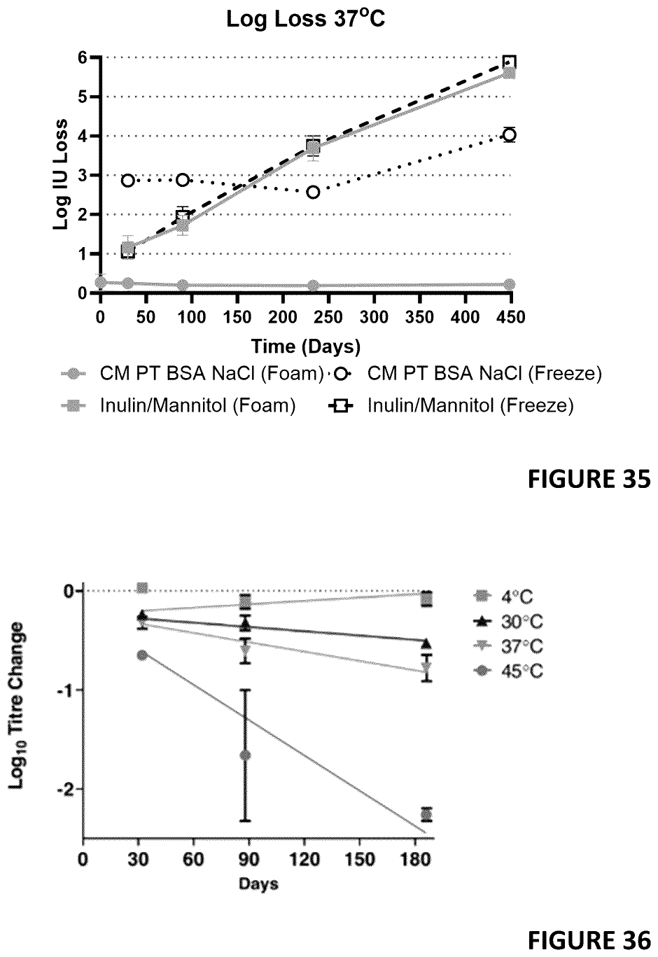

Before drying, each sample had about 10⁸ infectious units (IU). The dried samples were stored at 37° C. and tested for IU using an in-cell Western (ICW) assay with HEK293 cells. IU loss relative to the initial (before buffer exchange) IU of each formulation after drying (DO) and after storage for various time periods is shown in FIGS. 35-36.

IU loss relative to the initial (before buffer exchange) IU of each formulation after storage at 37° C. for 448 days was, foam dried Formulation CM PT BSA NaCl: 0.2 log loss and Formulation Inulin/Mannitol: 5.6 log loss. For freeze dried Formulation CM PT BSA NaCl: 4.0 log loss and Formulation Inulin/Mannitol: 5.9 log loss. Foam dried CM PT BSA NaCl is stable at 37° C. Foam dried CM PT BSA NaCl was stable at 37° C.

### Example 34

AdV-GFP was transferred from its stock buffer into a sample buffer by buffer exchange using 0.5 mL Zeba columns. 10 parts by volume of the sample buffer was mixed with 90 parts by volume of a solution of additional excipients dissolved in an additional amount of the sample buffer, as shown in Table 18 below.

TABLE 18
Formulations for The Study

| | | | (C) CM | (D) CM | (E) CM |
|---|---|---|---|---|---|
| | (A) CM | | PT BSA | PT BSA | PT BSA |
| | PT BSA | (B) | NaCl + | NaCl + | NaCl + |
| | NaCl | Tris/PT | Sorbitol | PS80 | PVP K15 |
| Tris (pH 7.2) | 10 mM | 10 mM | 10 mM | 10 mM | 10 mM |
| MgS0₄ | 10 mM | | 10 mM | 10 mM | 10 mM |
| CaCl₂ | 10 mM | | 10 mM | 10 mM | 10 mM |
| Gelatin | 0.05% | | 0.05% | 0.05% | 0.05% |
| BSA | 0.5% | | 0.5% | 0.5% | 0.5% |
| NaCl | 50 mM | | 50 mM | 50 mM | 50 mM |
| Pullulan | 2.5% | 2.5% | 2.5% | 2.5% | 2.5% |
| Trehalose | 5% | 5% | 5% | 5% | 5% |
| Sorbitol | | | 2% | | |
| PS-80 | | | | 0.5% | |
| PVP K15 | | | | | 5% |

The samples were foam dried. 100 uL samples of each formulation were placed in vials and pre-cooled to 4° C.. The pre-cooled vials were transferred to a lab scale freeze dryer operated as foam dryer. The pressure setpoint was 16uBar pressure. The temperature schedule was 10 hours at 400, 6.5 hours of a gradient from 400 to 2500, and 6.5 hours at 2500.

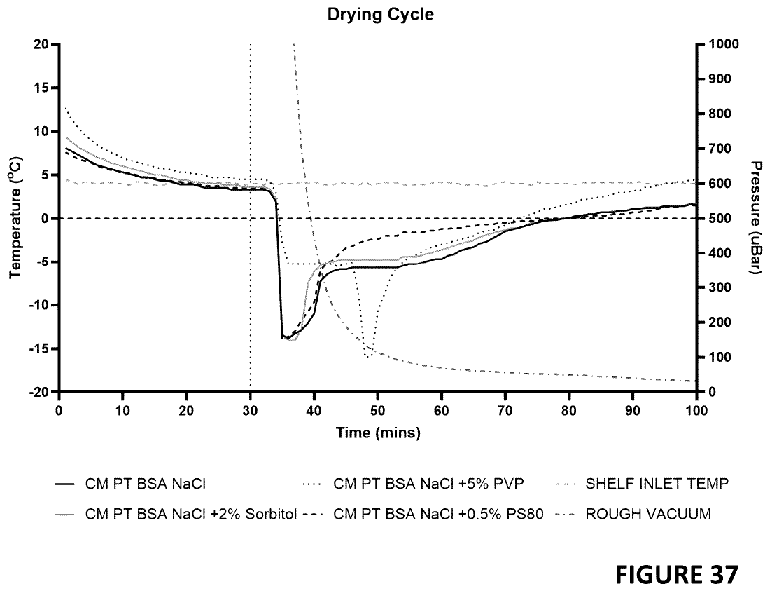

SP Dryer probe temperatures are shown on FIG. 37. Formulations A and C had very similar thermal traces. Both PS80 (formulation 0) and PVP (formulation E) were different: PS80 rose in temp quicker. PVP had delayed but greater temp drop, and rose to warmer afterwards.

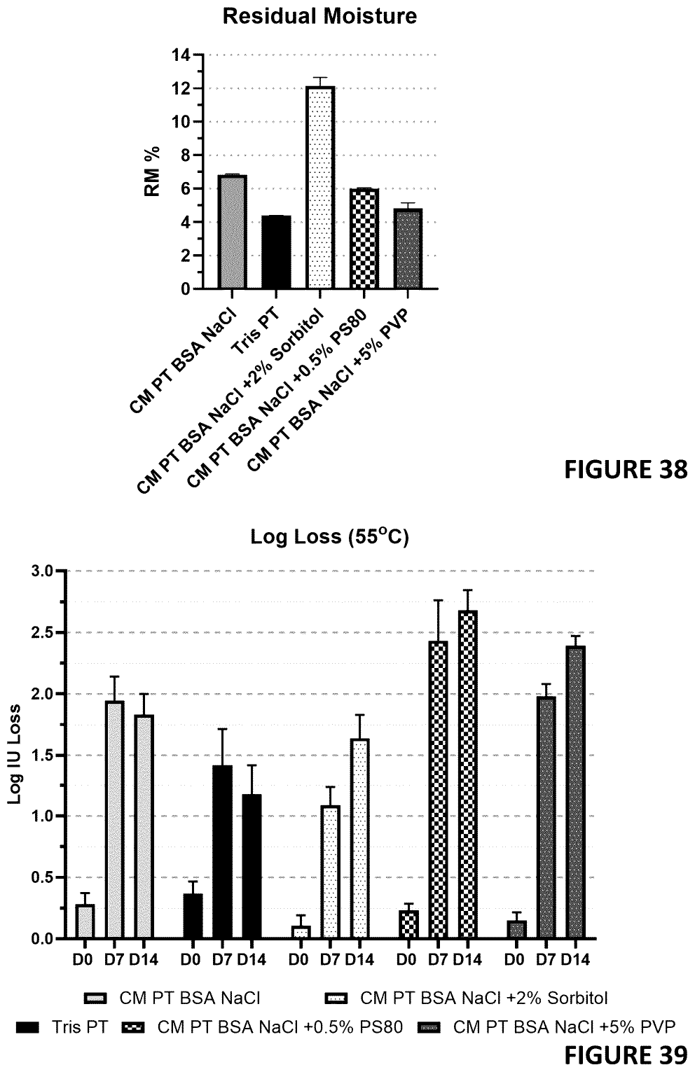

After foam drying, the residual moisture (RM) of Formulation A was 6.8%. The RM of Formulation B was 4.4%, Formulation C was 12.2%, Formulation 0 was 6.0% and Formulation E was 4.8% (FIG. 38).

Before drying, each sample had about 10⁸ infectious units (IU). The dried samples were stored at 55° C. and tested for IU using an in-cell Western (ICW) assay with HEK293 cells. IU loss relative to the initial (before buffer exchange) IU of each formulation after drying (DO) and after storage for various time periods is shown in FIG. 39.

IU loss relative to the initial (before buffer exchange) IU of each formulation after storage at 55° C. for 14 days was, Formulation A: 1.8 log loss; Formulation B: 1.2 log loss; Formulation C: 1.6 log loss, Formulation D: 2.7 log loss, and Formulation E: 2.4 log loss.

### Example 35

AdV-GFP was transferred from its stock buffer into a sample buffer by buffer exchange using 0.5 mL Zeba columns. 10 parts by volume of the sample buffer was mixed with 90 parts by volume of a solution of additional excipients dissolved in an additional amount of the sample buffer, as shown in Table 19 below.

TABLE 19
Formulations for The Study

| | | (B) | | |
|---|---|---|---|---|
| | | Tris/PT + | (C) Tris + | (D) CM PT BSA |
| | (A) Tris/PT | Sorbitol | Sorbitol | NaCl + Sorbitol |
| Tris (pH 7.2) | 10 mM | 10 mM | 10 mM | 10 mM |
| MgS0₄ | | | | 10 mM |
| CaCl₂ | | | | 10 mM |
| Gelatin | | | | 0.05% |
| BSA | | | | 0.5% |
| NaCl | | | | 50 mM |
| Pullulan | 2.5% | 2.5% | | 2.5% |
| Trehalose | 5% | 5% | | 5% |
| Sorbitol | | 2% | 2% | 2% |

The samples were foam dried. 100 uL samples of each formulation were placed in vials and pre-cooled to 4° C. The pre-cooled vials were transferred to a lab scale freeze dryer operated as foam dryer. The pressure setpoint was 16uBar pressure. The temperature schedule was 10 hours at 4° C., 6.5 hours of a gradient from 4° C. to 25° C., and 6.5 hours at 25° C.

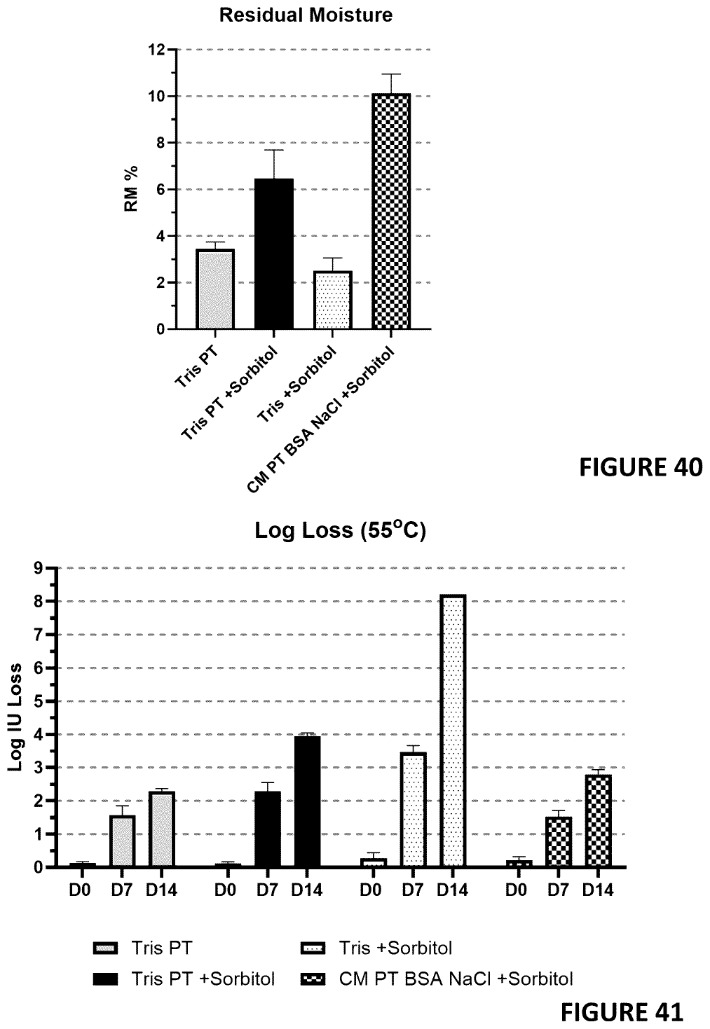

After foam drying, the residual moisture (RM) of Formulation A was 3.5%. The RM of Formulation B was 6.5%, Formulation C was 2.5%, and Formulation D was 10.1% (FIG. 40).

Before drying, each sample had about 10⁸ infectious units (IU). The dried samples were stored at 55° C. and tested for IU using an in-cell Western (ICW) assay with HEK293 cells. IU loss relative to the initial (before buffer exchange) IU of each formulation after drying (DO) and after storage for various time periods is shown in FIG. 41.

IU loss relative to the initial (before buffer exchange) IU of each formulation after storage at 55° C. for 14 days was, Formulation A: 2.3 log loss; Formulation B: 3.9 log loss; Formulation C: 8.2 log loss, and Formulation D: 2.8 log loss.

### Example 36

AdV-GFP or Ad-Luciferase (Luc) was transferred from its stock buffer into a sample buffer by buffer exchange using 0.5 mL Zeba columns. 10 parts by volume of the sample buffer was mixed with 90 parts by volume of a solution of additional excipients dissolved in an additional amount of the sample buffer, as shown in Table 20 below.

TABLE 20
Formulations for The Study

| | | | | Tris | |
|---|---|---|---|---|---|
| | | Tris | Tris | BSA/PT + | Tris BSA |
| | Tris | BSA/PT + | BSA/PT + | PMAL-16 + | 2.5% P |
| | BSA/PT | EDTA | PMAL-16 | EDTA | 15% T |
| | Concentration | | | | |
| Tris | 10 mM (pH 7.2) | | | | |
| BSA | 0.5% | | | | |
| EDTA | | 100 uM | | 100 uM | |
| PMAL-16 | | | 1% | 1% | |

| Pullulan | 2.5% | |
|---|---|---|
| Trehalose | 5% | 15% |

The samples were foam dried. 100 uL samples of each formulation were placed in vials and transferred to a lab scale freeze dryer operated as foam dryer. The pressure setpoint was 16uBar pressure. The temperature schedule was 23.5 hours at 25° C.

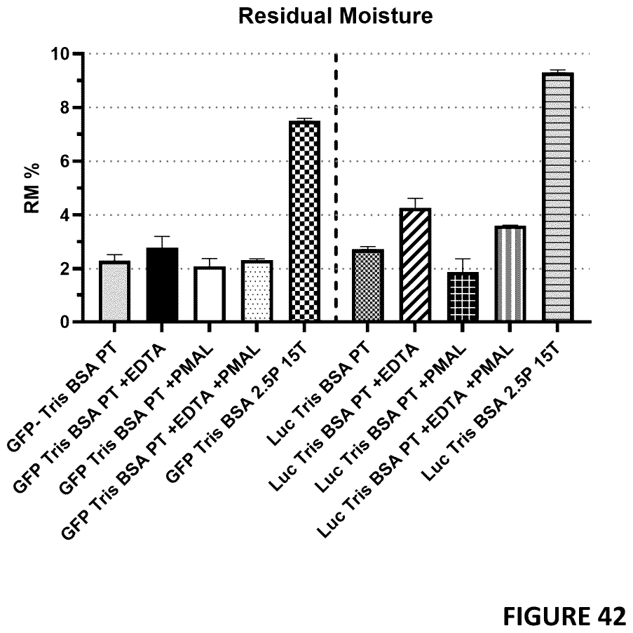

After foam drying, the residual moisture (RM) of Formulation A was 2.3% GFP, 2.7% Luc. The RM of Formulation B was 2.8% GFP/4.3% Luc, Formulation C was 2.1% GFP 1.9% Luc, Formulation D was 2.3% GFP/3.6% Luc, and Formulation E was 7.5% GFP/9.3% Luc (FIG. 42).

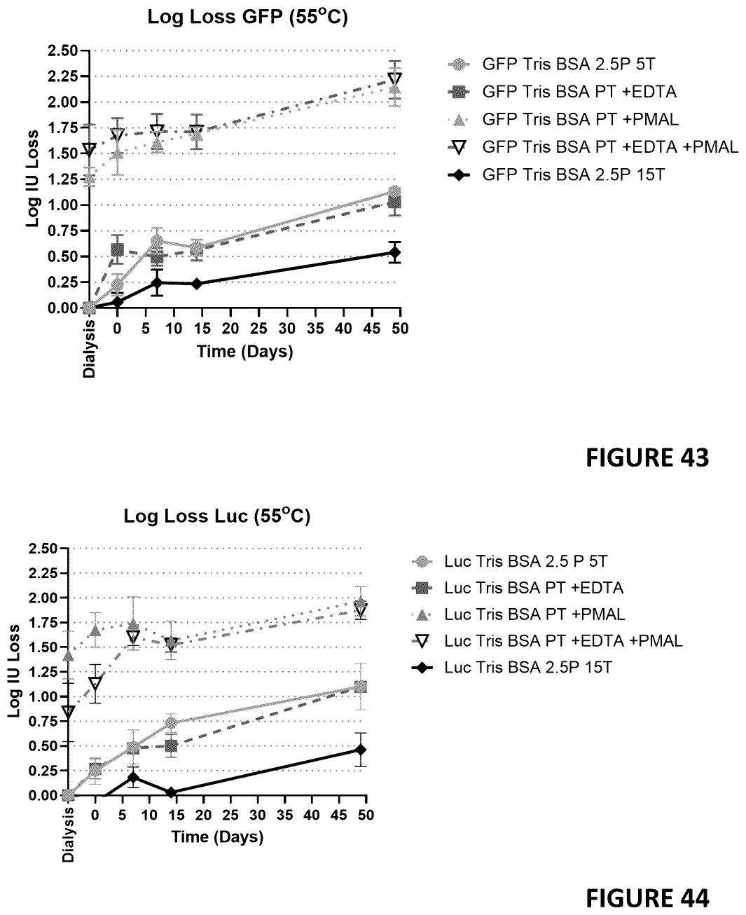

Before drying, each sample had about 10⁸ infectious units (IU). The dried samples were stored at 55° C. and tested for IU using an in-cell Western (ICW) assay with HEK293 cells. IU loss relative to the initial (before buffer exchange) IU of each formulation after drying (DO) and after storage for various time periods is shown in FIG. 43.

IU loss relative to the initial (before buffer exchange) IU of each formulation after storage at 55° C. for 49 days was, for AdV-GFP: Formulation A: 1.1 log loss; Formulation B: 1.0 log loss; Formulation C: 2.1 log loss, Formulation D: 2.2 log loss, and Formulation E: 0.5 log loss.

IU loss relative to the initial (before buffer exchange) IU of each formulation after storage at 55° C. for 49 days was, for AdV-Luc: Formulation A: 1.1 log loss; Formulation B: 1.1 log loss; Formulation C: 2.0 log loss, Formulation D: 1.9 log loss, and Formulation E: 0.5 log loss (FIG. 44).

### Example 37

HuAdV-GFP or a ChAdV-vectored Covid vaccine were transferred from their stock buffer into a sample buffer by buffer exchange using 0.5 mL Zeba columns. 10 parts by volume of the sample buffer was mixed with 90 parts by volume of a solution of additional excipients dissolved in an additional amount of the sample buffer. The same formulation concentration was used for each condition of 10 mM Tris pH 7.2, 0.5% BSA, 2.5% pullulan, and 15% trehalose (Table 21 below). Different lots of pullulan were used, and conditions with filtering occurred after all excipients were added. The expired pullulan was hypothesized to possibly have a lower molecular weight due to degradation. Filtration was also hypothesized to effect molecular weight.

TABLE 21
Formulations for the Study

| | | | |
|---|---|---|---|
| | (B) Matched | (C) Matched | (D) ChAd |
| (A) Expired | Lot Pullulan | Lot Pullan | COVID |
| Pullulan | No Filter | Filtered | Vaccine |

| | Concentration |
|---|---|
| Tris | 10 mM (pH 7.2) |
| BSA | 0.5% |
| Pullulan | 2.5% |
| Trehalose | 15% |

The samples were foam dried. 100 uL samples of each formulation were placed in vials and transferred to a lab scale freeze dryer operated as foam dryer. The pressure setpoint was 16uBar pressure. The temperature schedule was 23.5 hours at 25° C.

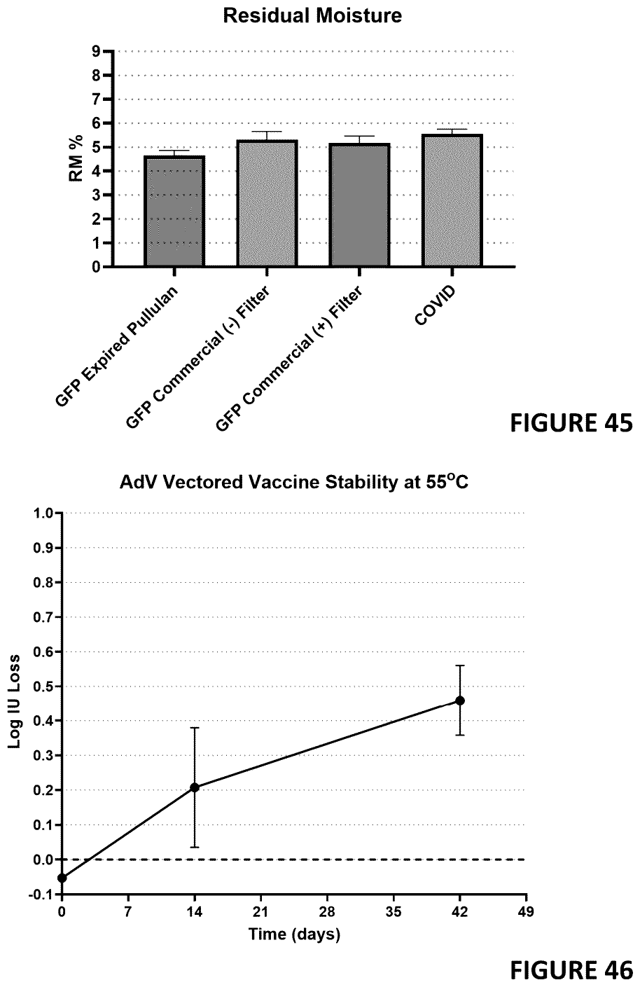

After foam drying, the residual moisture (RM) of Formulation A was 4.7%. The RM of Formulation B was 5.3%, Formulation C was 5.2%, and Formulation D was 5.6% (FIG. 45).

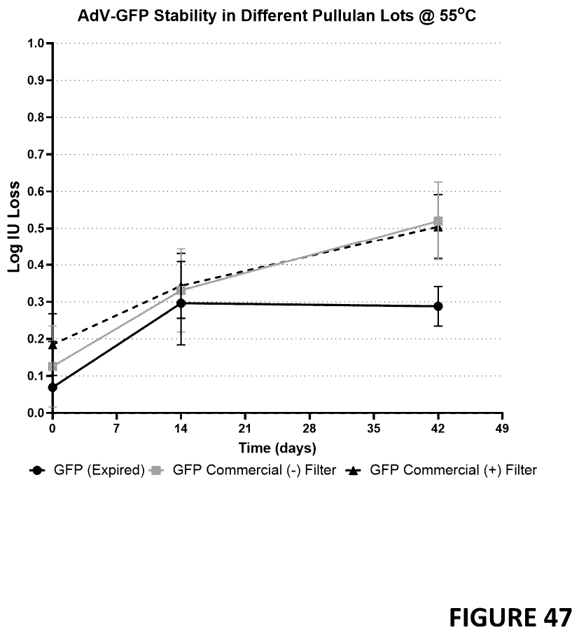

Before drying, each sample had about 10⁸ infectious units (IU). The dried samples were stored at 55° C. and tested for IU using an in-cell Western (ICW) assay with HEK293 cells. IU loss relative to the initial (before buffer exchange) IU of each formulation after drying (DO) and after storage for various time periods is shown in FIGS. 46-47.

IU loss relative to the initial (before buffer exchange) IU of each formulation after storage at 55° C. for 42 days was, Formulation A: 0.3 log loss; Formulation B: 0.5 log loss; Formulation C: 0.5 log loss, and Formulation D: 0.5 log loss. A 0.5 log loss at 55° C. is predicted to provide a 0.5 log loss after storage for 11 years at 25° C.

### DISCUSSION

Air drying does not provide stability to VSV; Freeze drying with trehalose alone does not stabilize, but pullulan and trehalose provides improved protection

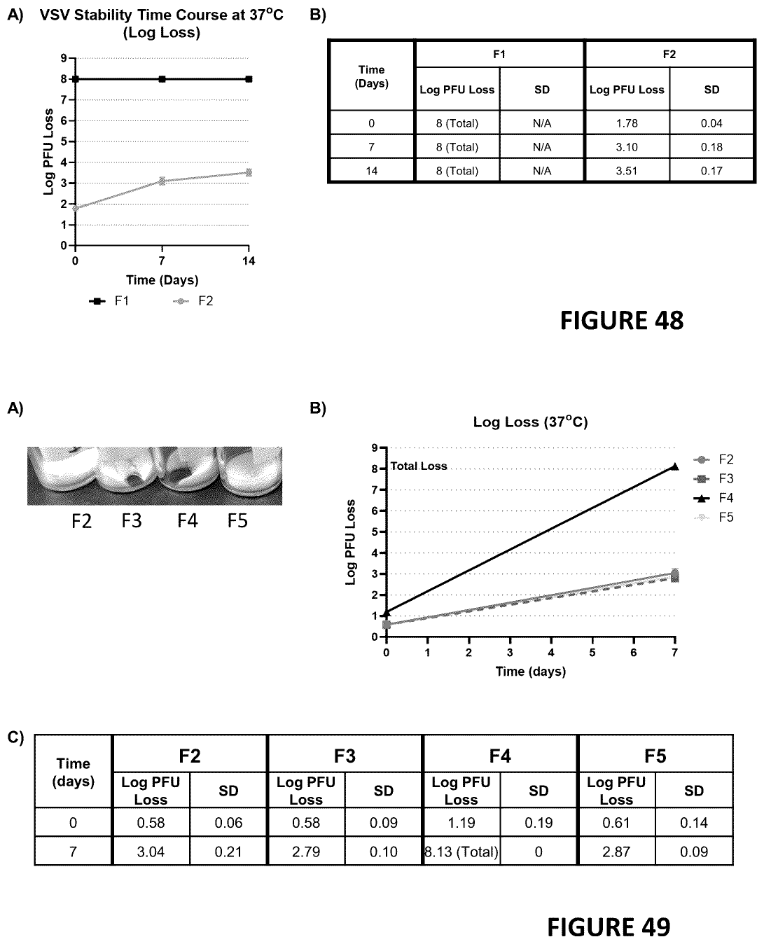

To the best of applicant's knowledge, the best thermal stability data published to date demonstrated a 4-log decrease in VSV titer after spray drying and thermal treatment at 37° C. for 7 days when formulated with trehalose [Toniolo, S. P., et al., Spray dried VSV-vectored vaccine is thermally stable and immunologically active in vivo. Sci Rep, 2020. 10(1): p. 13349]. Previous work demonstrated thermal stability of viral vaccines by air drying with pullulan and trehalose [Leung, V., et al., Thermal Stabilization of Viral Vaccines in Low-Cost Sugar Films. Sci Rep, 2019. 9(1): p. 7631]. To assess if the air-dried methodology (Table 22) could stabilize VSV, the virus in two different formulations was prepared and the process loss and thermal stability at 37° C. were tested (FIG. 48). The formulation utilized in Leung et al. (Table 23 below; formulation F1) had complete loss of titer during the drying process, and formulation F2 had high process loss of 1.78±0.04 Log PFU. After 14-day incubation at 37° C., a total PFU loss for VSV in F2 was 3.5±0.17 log, making it clear air drying was not a viable strategy for stabilizing VSV.

TABLE 22
Summary of Drying Schedules

| Schedule | | Pre- | | |
|---|---|---|---|---|
| Name | System | Incubation | Dry Schedule | Backfill |
| Air Dried | Fume Hood | Room | 72 hrs @ 25° C. with atmospheric pressure | N/A |
| | | Temperature | | |
| Vacuum | Vacuum Pump + 20 L | Room | 24-48 hrs @ 25° C. under 1e−4 mBar | Atmosphere; |
| | Bell Desiccator | Temperature | pressure | Manual Crimp |
| 25° C. | SP Virtis AdVantage | 25° C. | 24 hrs @ 25° C. under 16 uBar pressure | Atmosphere; |
| | Pro | | | Manual Crimp |
| Freeze Dry | | −50° C. | 10 hrs @ −50° C.; 6 hr ramp to 25° C.; 5 hr @ | Atmosphere; |
| | | | 25° C. under constant 16 uBar pressure | Manual Crimp |
| 4° C. | | 4° C. | 21 hrs @ 4° C. with 16 uBar pressure | Atmosphere; |
| | | | | Manual Crimp |
| 4/25° C. | | 4° C. | 10 hrs @ 4° C., 5 hrs ramp to 25° C., 6 hrs at | Atmosphere and |
| | | | 25° C. under constant 16 uBar pressure | Manual Crimp |
| 4/37° C. | | 4° C. | 10 h at 4° C., 6.5 hours ramp to 37° C., 4 hrs | Atmosphere; |
| | | | 37° C. under constant 16 uBar pressure | Manual Crimp |

Next experiment was to understand if a standard freeze-drying schedule (Table 22) with formulation F3 or trehalose alone (F4) could outperform the published data (FIG. 49). Visual observation of the dried film revealed F2, and F5 films had cake-like appearances, whereas F3 (higher concentration of pullulan and trehalose) and F4 led to a collapsed cake structure (FIG. 49A). For the three formulations with pullulan and trehalose, no significant difference was observed for process loss, and total log titer loss after 7 days at 37° C. (FIG. 49B). Interestingly, trehalose alone (F4) performed the worst, with the highest process loss and a complete loss of detectable plaques after 7 days at 37° C.. Despite the improvement of 1 log titer in stability of the freeze dry methodology after 7 days at 37° C. relative to spray drying, we did not view ˜3 log titer loss of formulated VSV as acceptable, and therefore transitioned to exploring vacuum drying methodologies for further process improvement.

TABLE 23
Summary of formulation components and concentrations used in this study.

| | Components | | | | | | | | |
|---|---|---|---|---|---|---|---|---|---|
| Formulation | Tris (pH 7.2) | CaCl₂ | MgSO₄ | Gelatin | BSA | NaCl | Pullulan | Trehalose | Other |
| F1 | | | | | | | 10% | 20% | |
| F2 | 10 mM | 10 mM | 10 mM | 0.05% | 0.5% | 50 mM | 2.5% | 5% | |
| F3 | 10 mM | 10 mM | 10 mM | 0.05% | 0.5% | 50 mM | 5% | 10% | |
| F4 | 10 mM | | | | | | | 5% | |
| F5 | 10 mM | | | | 0.5% | 50 mM | 2.5% | 5% | |
| F6 | 10 mM | 10 mM | 10 mM | 0.05% | | | | | |
| F7 | 10 mM | 10 mM | 10 mM | 0.05% | | | | 10% | |
| F8 | 10 mM | 10 mM | 10 mM | 0.05% | | | 5% | 10% | |
| F9 | 10 mM | 10 mM | 10 mM | 0.05% | | | 2.5% | 5% | |
| F10 | 10 mM | | | 0.05% | 0.5% | | 2.5% | 5% | |
| F11 | 10 mM | | | | | | | | 0.5% Gelatin |
| | | | | | | | | | 2% Sorbitol |
| F12 | | | | | | | | 5% | |
| F13 | 10 mM | | | | 0.5% | | 2.5% | 5% | |

For powdered components, the percentage is calculated as w/v.
Vacuum Drying at 25° C. has high process loss with formulation F7, but no thermal stability loss over 14-day incubation at 37° C.

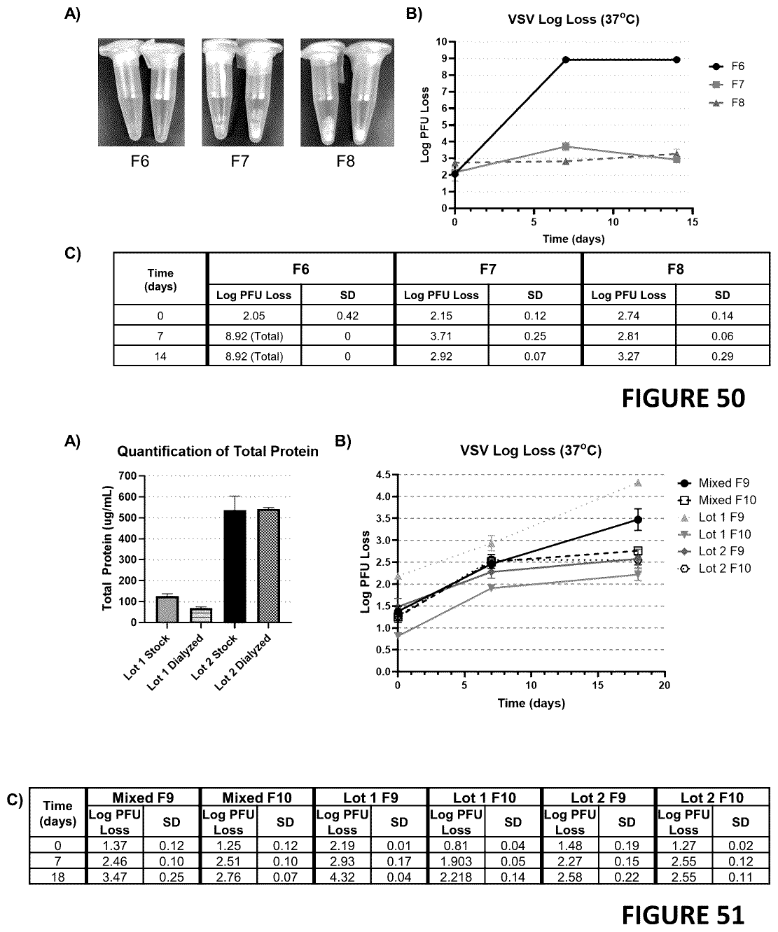

Unlike freeze drying, vacuum drying does not induce stresses such as freezing and/or ice crystals potentially damaging the VSV lipid membrane. It was assessed if drying VSV in formulation F7/F8 at room temperature with a vacuum pump could improve process loss and thermal stabilization (FIG. 50). Samples were dried for 48 hours at room temperature under a vacuum pressure of 1e⁴ mBar (Table 22). Visual observation of VSV dried in pullulan and/or trehalose (F7/F8) had a bubbly foam architecture, whereas VSV dried in buffer alone (F6) did not yield a film (FIG. 50A). All three conditions had significant process loss of VSV titer due to drying, with formulation F6 providing no recoverable plaques after 7 days incubated at 3700 (FIG. 50 B/C). Interestingly, both F7 and F8 films did not show significant additional thermal challenge loss over two weeks at 3700. This observation of VSV being thermally stable once dried in a film led the inventors to explore different mechanisms to improve the drying process with additional excipients and improvements to the drying schedule.

Addition of serum albumin significantly improves stability of VSV in a lot-to-lot dependent manner

Several experiments were conducted testing different pH's (6.8-8.1), concentration of Tris buffer (between 10-50 mM), and excipients including hydroxyectoine, ectoine, β-cyclodextran, PEG 200, PEG 4000, PEG 6000, Histidine, Glutamic acid, and increased gelatin concentrations with no improvement in VSV stabilization. We also observed no difference in process loss or thermal stability of vacuum dried VSV between 24 and 48 hr dry schedules.

The addition of serum albumin, specifically bovine serum albumin (BSA), improved stability of VSV in a lot-to-lot dependent manner (FIG. 51). Two different lots of purified VSV had significantly different total protein content as determined by Bradford assay both pre and post dialysis into the formulation buffer despite no significant difference in the titer of the viral stock (3e¹⁰ vs 4e¹⁰ PFU/mL; FIG. 51A). It was hypothesized that the difference is due to cell culture carryover from the purification protocol. Analysis of the process loss of the conditions tested revealed VSV lot 1 (low total protein) had significantly more process loss in the absence of BSA (F9) compared to the addition of 0.5% (FIG. 51B). VSV lot 2 (high total protein) showed no difference in process loss with (F10) or without BSA (F9). For the samples with the two lots mixed at a 1:1 ratio, no significant difference was observed between formulation F9 and F10 for process loss. After 18 days incubation at 37° C., the VSV lot 1 samples had ˜2 log difference between +/− BSA, the Lot 2 samples had no difference, and the mixed group was improved by ˜0.7 logs with the addition of BSA. VSV manufactured in GMP processes are typically done in FBS-free conditions, resulting in less protein carryover during the purification of the viral vector. These results led to the conclusion that the addition of serum albumin, and in this case BSA, improves stability of VSV in low total protein conditions.

Long term stability of VSV in F2 formulation at 37° C. demonstrates stable titer after initial loss

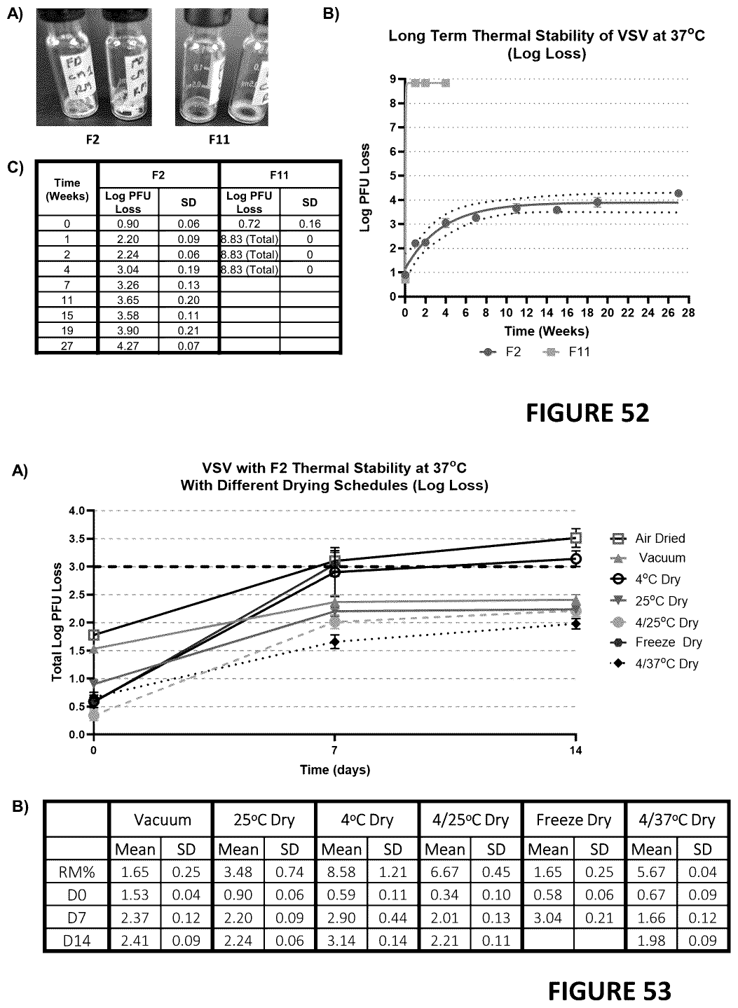

To determine the long-term thermal stability of the F2 formulation for VSV, an AdVantage Pro drying system was used for greater control of the foam drying schedule. As described in previous literature, most viral vectors are formulated and dried with sorbitol and gelatin, which were employed as a control formulation [Hansen, L. J. J., et al., Freeze-drying of live virus vaccines: A review. Vaccine, 2015. 33(42): p. 5507-5519.]. Samples were dried for 24 hours at 25° C. at a pressure of 16 uBar, manually stoppered and crimped (FIG. 52). F2 formulation dried samples were observed to be a bubbly film, whereas the gelatin/sorbitol samples were a flatter and more uniform glassy film (FIG. 52A). Both formulations had similar process loss (FIG. 52B/C). After 1 week incubation at 37° C., no viable VSV plaques were observed for the F11 samples and were confirmed for week 2 and 4 samples. Conversely, F2 samples showed a total log loss of 2.2 for 1 and 2-week samples, and 3.0±0.19 log loss at 4 weeks at 37° C. Interestingly, it was observed from weeks 4 to 27 a smaller additional log loss of VSV titer, resulting in a total loss at 27 weeks of 4.27±0.07 log VSV at 37° C. Modelling of the VSV in formulation F2 with a one phase decay non-linear regression model results in a R² value of 0.94 and a 95% confidence interval of the plateau between 3.84-4.60 log PFU loss. These data suggest the trehalose and pullulan-based formulations, once stable, have limited additional loss of VSV titer.

Two stage temperature drying schedule improves process loss and short-term stability at 37° C.

Next it was sought to improve the drying schedule of foam drying the PT formulation to improve VSV stability. Several different drying schedules were assayed and compared with the F2 formulated VSV (FIG. 53). Of the conditions tested, the two most total log loss by day 14 were obtained in the freeze dry schedule and drying for 24 hours at 4° C. under 16 pBar pressure. The schedule that had the least amount of process loss was a 24-hour dry schedule (Table 22). Comparable total log losses were observed with alternative schedules, but it was decided to continue optimizing the VSV stability work with the 4/25° C. schedule due to the lowest observed process loss.

Long-term stability with 4/25° C. dry schedule has less titer loss with Formulation F2 compared to drying at 25° C. alone at 37° C. thermal challenge; significantly less PFU loss at 4° C.

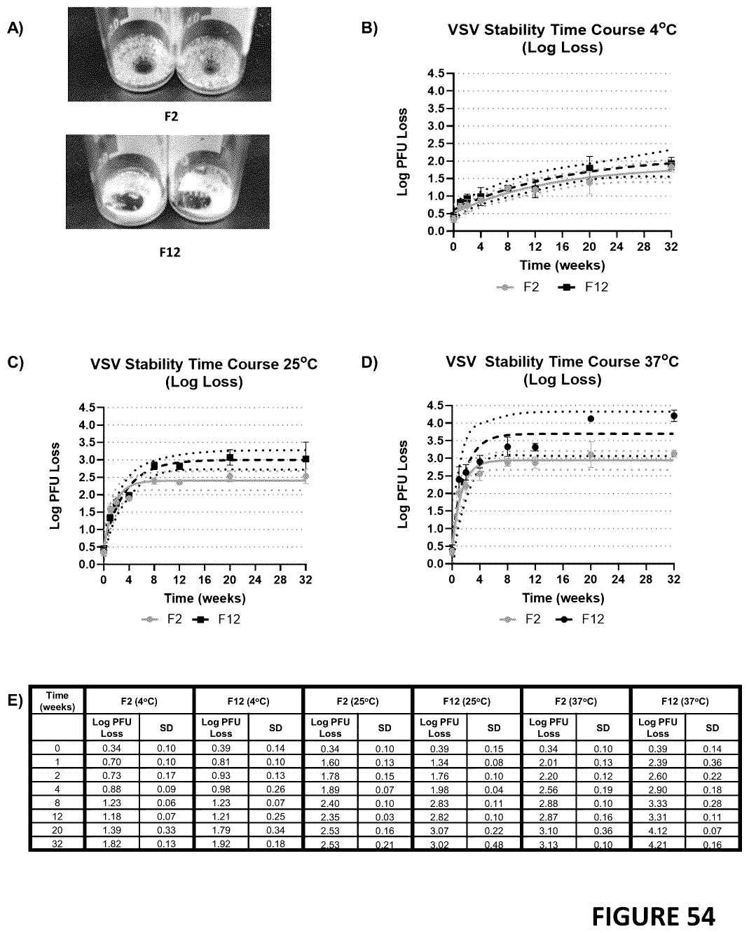

Using the optimized drying schedule, the long-term stability for 32 weeks of the formulations at 4° C., 25° C., and 37° C. were assayed (FIG. 54). Two formulations, F2 and F12, dried over 24 hours with 4/25° C. dry schedule which were manually stoppered and crimped and compared (FIG. 54A). Both formulations had comparable process losses and both statistically lower than F2 dried with 25° C. alone dry schedule (P=0.001 and P=0.002 respectively). For the thermal stability test at 4° C. (FIG. 54B), the titer loss for both formulations increased to 1.23±0.07 by 8 weeks, and at the end of 32 weeks total titer loss for F2 was 1.82±0.13 log and for F12 it was 1.93±0.18 log (P=0.29). Modelling with a one phase decay non-linear regression model results in a R² value of 0.93 for both F2 and F11. The calculated plateau for each formulation at 4° C. was 1.87 and 2.12 log PFU loss for F2 and F11 respectively.

For samples incubated at 25° C. (FIG. 54C), both formulations had significant titer loss up to week 8 with F2 outperforming F12 for total PFU loss (P=0.0021). Interestingly, for both formulations only a slight increase in loss was observed between week 8 and week 32 samples. At the conclusion of the experiment at 25° C., a total loss was observed for F2 was less than the loss observed for formulation F12 (P=0.0001). Modelling with a one phase decay non-linear regression model results in a R² value of 0.94 for F2 and 0.98 for F11. The calculated plateau for each formulation at 25° C. was 2.40 and 3.00 log PFU for F2 and F11 respectively.

A similar trend occurred for samples incubated at 37° C. (FIG. 54D). By 8 weeks, the F2 formulation total PFU loss was 2.88±0.10 log and F12 was 3.33±0.28 log, with the F2 dried with 4/25° C. outperforming both the F12 formulation and F2 dried at 25° C. (P=0.0012 and P=0.0018 respectively). After 32 weeks at 37° C., the F2 formulation total loss was 3.13±0.10 log but significantly more loss was observed for F12 (4.20±0.16 log; P<0.0001). For the 25° C. dry schedule, formulation F2 total log loss was 4.3±0.07 at week 27, like the loss observed for F12 at 32 weeks. Modelling with a one phase decay non-linear regression model results in an R² value of 0.96 for F2 and 0.88 for F11. The calculated plateau for each formulation at 37° C. was 2.94 and 3.69 log PFU for F2 and F11 respectively. The calculated plateau for F11 was significantly less log loss compared to the data collected, suggesting additional factors may be involved resulting in greater VSV titer loss in this formulation.

Overall, no significant difference was observed between the two formulation's stability time course at 4° C., but the F2 outperformed the F12 at 25° C. and 37° C. with the 4/25° C. dry schedule as well as outperforming the 25° C. only dry schedule.

Backfilling vials of dried formulation F2 with dry Nitrogen gas causes crystallization to occur in film decreasing viability of VSV

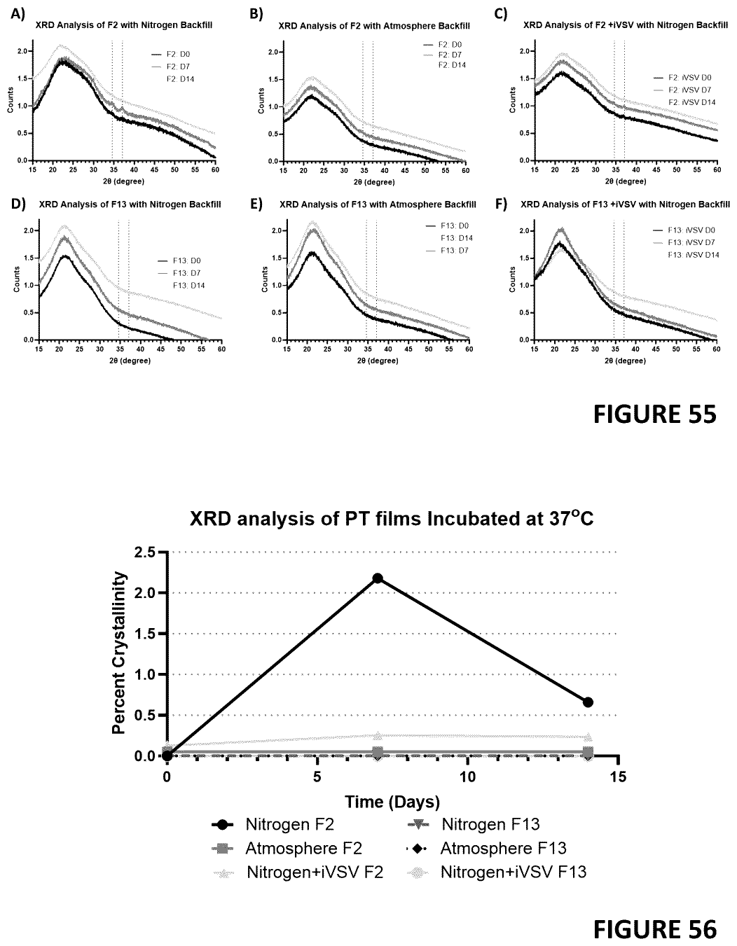

For commercial dried products, it is commonplace to backfill samples with an inert gas like nitrogen to improve stability by reducing chemical instabilities [Lai, M.C. and E.M. Topp, Solid-state chemical stability of proteins and peptides. J Pharm Sci, 1999. 88(5): p. 489-500.]. Over several experimental tests with nitrogen backfilling, significantly greater loss of VSV titer was observed compared to the data demonstrated in both FIG. 53 and FIG. 54. To understand if the nitrogen backfilling was affecting the structure of the dried film, XRD analysis was performed on a thermal treated time course at 37° C. (FIG. 56). All conditions assayed were completely amorphous on day 0 before incubation at 37° C. (FIG. 55). After 7 days incubations 2.2% crystallinity was detected in the F2 films when backfilled with nitrogen gas, but no crystallinity when backfilled with atmospheric air. The crystallinity percentage decreased by 14 days incubation to 0.6% in the nitrogen filled vials. According to the XRD trace profiles, it was hypothesized that calcium sulfate crystals are the causative material for the observed crystallinity. Samples comprised of formulation F13 were completely amorphous for the time course independent of backfilling gas. An interesting observation of samples with UV inactivated VSV had a constant lower crystallinity from Day 0 to Day 14 of the experiment (˜0.2%) in the F2 formulation with nitrogen backfilling but completely amorphous with F13. These results led to the conclusion in the absence of moisture in the headspace gas, crystallization can occur with the ions present in the formulation, and this stress decreases the stability of VSV.

Nitrogen backfilled vials with formulation F13 improves serum free manufactured VSV stability over atmospheric backfilled formulation F2 modestly at 4° C. and significantly at 25° C. and 37° C.

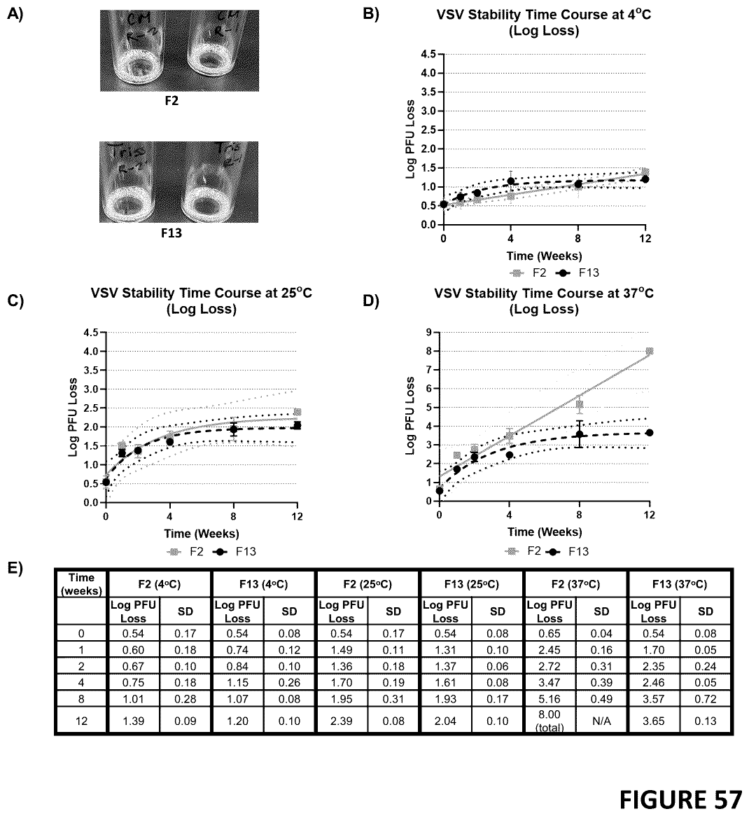

To determine if a combination of nitrogen backfilled vials and removing all ions from the formulation would improve the VSV stability, two formulations, F2 and F13 were dried with the 4/25° C. dry schedule and backfilled with dried Nitrogen gas (FIG. 57). For this experiment, VSV was manufactured in a serum free media to simulate a GMP process [25]. Both dried formulations had a similar bubbly dried appearance (FIG. 57A) characteristic of a foam dried material. Both formulations had nearly identical process loss. After 8-week incubation at 4° C., both formulations had very similar total log loss for F13 and F2 (FIG. 57B) and are not statistically different from the F2 containing atmospheric air in the vial headspace (FIG. 57B). Similarly, the two formulations incubated at 25° C. did not have any difference in stability at 8 weeks (FIG. 57C), with both formulations outperforming the F2 with atmospheric air headspace formulation after 8 weeks incubation (2-way ANOVA; F13 P-value=0.009, F2 nitrogen P-value=0.012). Conversely, the F13 formulation significantly outperformed the F2 nitrogen backfilled vials at 37° C. after 8-week incubation (FIG. 57D). No statistical difference was observed between the atmosphere air filled F2 samples and the nitrogen filled F13 samples (P=0.35) but the nitrogen filled F2 samples were significantly lower titer (P=001). Interestingly, after a 12-week incubation period, greater statistical differences were observed between the formulations. At 4° C., no difference in loss was observed between the nitrogen backfilled F13 and atmosphere backfilled F2, but both outperformed the nitrogen backfilled F2 when compared with one-way ANOVA (P<0.0001 and P=0.0002 respectively). Modelling with a one phase decay non-linear regression model results in a R² value of 0.98 for F2 and 0.94 for F13. The calculated plateau for each formulation at 4° C. was 1.17 log PFU loss for F13, but surprisingly no stable plateau was determined for F2. Samples incubated at 25° C. demonstrated nitrogen backfilled F13 statistically outperformed both nitrogen backfilled F2 and atmosphere backfilled F2 (one-way ANOVA; both P<0.0001). Modelling with a one phase decay non-linear regression model results in an R2 value of 0.88 for F2 and 0.95 for F13. The calculated plateau for each formulation at 25° C. was 2.26 log PFU loss for F2, and 1.98 log PFU loss for F13. However, at 37° C., the best performing formulation after 12-week incubation was the atmosphere backfilled F2 compared to nitrogen backfilled F13 and nitrogen backfilled F2 which did not have any detectable PFU (one-way ANOVA P<0.0001). Modelling with a one phase decay non-linear regression model results in a R² value of 0.96 for F2 and for F13. The calculated plateau for each formulation at 37° C. was not able to be calculated for F2 and was 3.67 log PFU loss for F13. Overall, these data demonstrate that similar levels of stability can be achieved with nitrogen backfilling of F13 formulation to the atmospherically filled F2 formulation at 4° C. and 25° C., but atmosphere backfilled formulation F2 outperforms nitrogen backfilled F13 at 37° C. Results

We studied various different conditions to thermally stabilize an inherently unstable but therapeutically important viral vector VSV. Utilizing both air drying and freeze-drying methodologies did not yield satisfactory process or thermal stability losses for VSV, with losses of greater than 3 log PFU after 7-day incubation at 37° C. An improvement in stabilization was observed under vacuum drying conditions at room temperature, but the process loss was still problematic. We studied whether additional excipients might improve the stability of VSV during the drying process and initial two weeks of thermal stability at 37° C. While most excipients assayed showed no improvement in PFU recovery, one excipient identified was bovine serum albumin (BSA). BSA was used in the studied herein but would likely be replaced with a form of human albumin for safety in a vaccine. For example the ERVEBO vaccine that recombinant human serum albumin (rHSA) [CDC. Expanded Access Investigational New Drug (IND) Protocol: Ervebo® (Ebola Zaire Vaccine, Live) Booster Dose for Domestic Preexposure Prophylaxis (PrEP) Vaccination of Adults (>18 years of age) at Potential Occupational Risk for Exposure to Zaire ebolavirus. 2022, Zimmer, B., K. Summermatter, and G. Zimmer, Stability and inactivation of vesicular stomatitis virus, a prototype rhabdovirus. Vet Microbiol, 2013. 162(1): p. 78-84. It appears VSV is more sensitive to drying in the absence of additional proteins in the formulation, as was observed in a lot-to-lot dependent manner.

An exploration of different drying schedules with constant low pressure included a two stage method, starting at 4° C. followed by a second stage at 25° C. It was hypothesized that if purified VSV is inherently thermally unstable above ˜10° C. in solution, maintaining a VSV tolerable temperature until the water has been removed and the film or foam has formed would improve the process loss observed. A second drying stage at elevated temperatures is required to remove additional moisture from the film, improving the thermal stability of the formulation.

For the three long-term stability experiments described, improvements were observed with each optimization. Process loss was improved by optimizing the drying schedule from single 25° C. temperature to a two-stage process. This change in drying schedule also yielded a long-term thermal stability at 37° C. improvement of ˜1.3 log PFU after 27 weeks incubation. Changing the formulation to remove salt ions and backfilling the vial headspace has not shown any improvement in loss relative to formulation F2 with atmosphere backfilling, but significant improvement relative to F2 with Nitrogen backfilled vials after 12 weeks at 37° C. For all three long term experiments in this work, the vast majority of VSV titer loss was observed during the first 6-8 weeks of incubation at 37° C. While an additional 1 log loss is observed for 25° C. dry schedule from week 8 to week 27 (FIG. 52), VSV dried with formulation F2 and the 4/25° C. dry schedule had no statistical change in titer from week 8 until the completion of the experiment at week 32 (FIG. 54; P=0.257). This observed plateau also occurs at 25° C., at a lower loss of VSV titer. For F2 formulated VSV incubated at 4° C., a plateau was not observed in the data collected. It was hypothesized that since the stability of formulated VSV does not follow Arrhenius kinetics, different degradation mechanisms are occurring, and further investigation is warranted to elucidate the mechanisms at play. For the strategy of vaccine stockpiling for future disease outbreaks, being able to demonstrate a thermally stable plateau is a significant breakthrough for the VSV vaccine platform.

It is widely accepted that oxygen and moisture in the vial headspace cause decrease in the stability of the pharmaceutical agent [Buecheler, J. W., et al., Oxidation-Induced Destabilization of Model Antibody-Drug Conjugates. J Pharm Sci, 2019. 108(3): p. 1236-1245]. Initial characterization of the stability of VSV in dried pullulan and trehalose-based films were done with manual stoppering and crimping, resulting in atmospheric gas in the vial headspace. Contrary to the expectation, in transitioning the drying strategy to a more commercial based approach of backfilling with dried nitrogen gas with in-system stoppering, significantly higher VSV titer losses were observed at 37° C. (FIG. 57). Analysis by XRD of the dried formulation under different temperatures, timepoints, and backfilling gases revealed the presence of crystalline species (FIG. 56). Crystallinity was only observed at 37° C. in the presence of nitrogen backfilling with metal ions present with all other conditions being completely amorphous films (FIG. 55). While not as prevalent, a modest degree of crystallinity is detected when UV-inactivated VSV is present in the sample. It is hypothesized that the atmospheric humidity has sufficient moisture to prevent crystallization, whereas dried nitrogen gas has low excess moisture which promotes crystal formation. Crystallinity has been previously demonstrated to decrease the stability of several classes of pharmaceutical molecules [Lai, M. C. and E. M. Topp, Solid-state chemical stability of proteins and peptides. J Pharm Sci, 1999. 88(5): p. 489-500; Mensink, M. A., et al., How sugars protect proteins in the solid state and during drying (review): Mechanisms of stabilization in relation to stress conditions. Eur J Pharm Biopharm, 2017. 114: p. 288-295]. However, by removing all free ions from the formulation and backfilling with dried nitrogen gas, stability of VSV at elevated temperatures was improved.

In summary, it was demonstrated that by optimizing a pullulan and trehalose-based formulation, drying schedule, and vial backfilling, conditions to thermally stabilize rVSV vial vector were identified. These results are a significant improvement on any other described thermal stabilization methodology for rVSV.

### Example 38—Standard vs Gentle Drying

Formulation A from Example 34 (CM PT BSA NaCl) was dried in standard and Gentle drying. VSV samples were stored on ice while being formulated. During the formulation step the samples were at 25° C. for about 30 minutes and then transferred to the SPVirtis Advantage Pro freeze dryer.

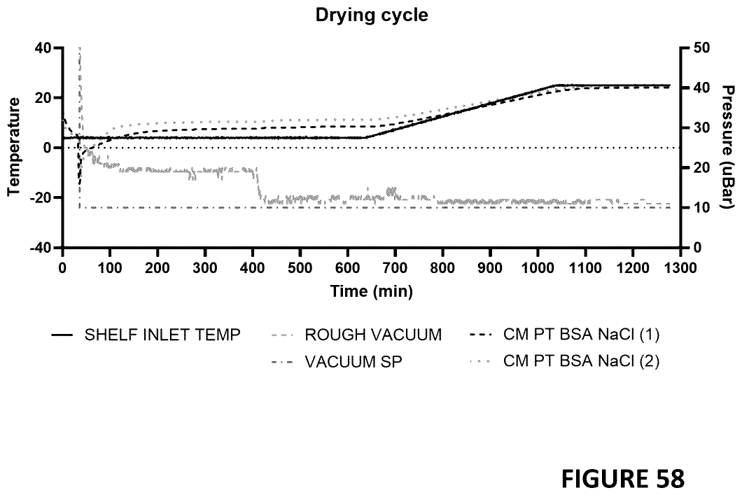

Standard drying (See pressure and temperature traces on FIG. 58):

VSV samples were placed on the shelf of the dryer which is set to 4° C. and allowed to equilibrate for 30 minutes. The vacuum was turned on (20 ubar) and the sample temperature dropped to ˜14° C. within 5 minutes of turning on the vacuum. After 8 minutes of having the vacuum on, the sample temperature began to rise indicating that it is a solid and fast evaporation has completed. After 60 minutes, the sample temperature reached the temperature indicating that there was no more significant loss of liquid.

Gentle Drying (See pressure and temperature traces on FIG. 23):

VSV samples were placed on the shelf of the dryer which is set to 8° C. No equilibration of the samples. The vacuum is turned on (20 mbar) and the sample temperature drops to around 5-6° C. (due to evaporative cooling) within the 15 minutes. Once the sample has dried the sample temperature starts to increase at about 90 minutes and reaches about the shelf temperature at 95 minutes.

### Materials and Methods

### Materials

Pullulan and trehalose SG dried powders were kindly provided by Nagase Viita Co., Ltd. Purified VSV serotype Indiana, with a AM51 mutation in the matrix gene (renders it interferon inducing and attenuated) stocks were manufactured and provided by Dr. Brian Lichty's research group (McMaster University) as previously described [29, 30]. Stock buffers Tris (1M pH 7.2; Teknova Cat No: T1072), 1M CaCl₂ (Sigma Cat No: 21115-100 mL), 1M MgSO₄ (Sigma; Cat No: M3409-100 mL), 5M NaCl (VWR; E529-500 mL), and dry powders of Bovine Serum Albumin (Sigma; A9418-10G) and Gelatin (Criterion, C7921) were diluted and resuspended as described in Table 1.

### VSV Dialysis and Formulation

Manufactured 100 μL VSV stock aliquots were stored at −80° C. in 4% sucrose. After gently thawing on ice, viral particles were buffer exchanged into the formulation buffer using Zeba Spin Desalting columns 7K MWCO (Thermo Scientific). Viral particles were mixed at a 1:9 ratio with the formulation buffer containing Pullulan and/or Trehalose and 100 μL was aliquoted into 2 mL vials. Samples were subsequently dried either using a vacuum pump and desiccator or the SP VirTis AdVantage Pro Freeze dryer system. If required, after completion of the dry schedule on the SP VirTis AdVantage Pro, vials were backfilled with dried Nitrogen gas and stoppered in system.

Residual Moisture Analysis with KF Titrator

Residual moisture of the dried VSV formulation was determined by KF titration with a Mettler-Toledo KF Titrator C10S system. The mass of the dried formulated VSV sample was calculated from pre-tared vials, 1 mL of Hydranal (Honeywell; Cat No 34724-1 L) was added to reconstitute film and reweighed to calculate mass of solvent. To determine background moisture content of Hydranal buffer, ˜0.6 mL of Hydranal stock was injected into the KF Titrator to determine PPM. The PPM of the dried film was determined by injecting 0.4 mL of reconstituted sample into the KF titrator. The residual moisture percentage of the dried film was calculated from the weight of the dried film, PPM of Hydranal blank, and the PPM calculated for the injected sample (Doan, 2022). Each formulation was analyzed in technical duplicates of biological replicates.

### VSV Plaque Assay for Viable PFU Quantification

Vero cells (derived from African green monkey kidney epithelial cells) were employed in 6 cm petri dishes to quantify PFU of VSV. The day prior to infection, Vero cells grown in 150 mm plates to 80-90% confluency were harvested using 3 mL of 1× Trypsin (Gibco, Life Technologies 15400-054) and incubated for 3 mins at 37° C.. Cells were resuspended, quantified, diluted to a concentration of 1.4×10⁶ cells/5 mL in completed media (α-MEM), plated in 6 cm dishes, and incubated overnight at 37° C./5% CO₂.

The next day, an overlay solution (1:1 ratio 1% Agar to completed media) was prepared and stored at 42° C. Dried formulated VSV samples were reconstituted with 1 mL MQH20 with light vortexing for 15 seconds prior to dilution in completed media. 100 μL of diluted VSV solution was pipetted onto Vero cells in 6 cm petri dishes for infection and rocked 3× for 15 mins (total 45 mins) at 37° C. Add 5 mL of overlay solution and allow to solidify before overnight incubation at 37° C./5% CO₂. Plaque count is performed the next day, and calculations were performed to determine the viable PFU of the formulated VSV.

UV-Inactivation of rVSV

To inactivate VSV for XRD analysis, a CL-1000 Ultraviolet Crosslinker (mercury lamp 254 nm wavelength) was employed. 125 μL of stock rVSV (1e¹⁰ PFU/mL) was added to a 24 well plate well and completely inactivated at 2000 mJ/cm². Inactivation was confirmed by plaque assay.

### XRD Analysis of Dried Formulations for Crystallinity

Dried formulations were prepared with the 4/25° C. dry cycle in 2 mL vials. For XRD analysis, the film was crushed into a powder and extracted from the vials. The powder samples were mounted on top of a zero-background Si wafer for the XRD measurement. Data were collected using a Bruker D8 DISCOVER with DAVINCI.DESIGN diffractometer, equipped with a Co sealed-tube source and Eiger2R 500K area detector. A 2D continuous scan from 12-80 degrees 2Th was collected with 0.02deg steps, at 1.2s per step.

### Statistical Analysis

Statistical analysis and graphing of data were performed with PRISM software (Graphpad Prism v10.1.2). Comparison of two groups was performed with two-tailed paired t-tests. For analysis of more than two unique groups, ANOVA was utilized with Tukey multiple analyses parameter.

We claim:

1. A method of preserving and/or stabilizing a virus, the method comprising:
combining the virus with trehalose, water and a buffer, optionally with carbohydrate and/or albumin to produce a liquid composition; and,
drying the liquid composition to produce a dried composition,
wherein drying the composition comprises removing a portion of the solvent from the composition under a pressure and a temperature sufficient to produce a moist solid within about 240 minutes of applying a vacuum to the liquid composition.

2. The method of claim 1 wherein the pressure and the temperature are sufficient to maintain the temperature of the composition above its freezing point.

3. The method of claim 1 wherein the liquid composition is not maintained at a temperature above 100° C. for more than 90 minutes.

4. The method of claim 1 wherein the virus is a live-attenuated virus and/or a viral vector.

5. The method of claim 1 wherein the virus is derived from a vesicular stomatitis virus (VSV).

6. The method of claim 1 wherein the virus is derived from an adenovirus (AdV).

7. The method of claim 1 wherein the dried composition is not foamed.

8. The method of claim 1 further comprising exposing the composition obtained after the step of removing the portion of the solvent to a vacuum while heating the composition, for example to a temperature in the range of 20° C. to 4° C..

9. The method of claim 1 wherein the pressure is in the range of 5 uBar to 100 mBar, 5 uBar to 50 mBar, 5 uBar to 10 mBar, 5 uBar to 5 mBar, 5-200 uBar or 5-20 uBar, 44.

10. The method of claim 1 wherein the composition comprises albumin, for example in an amount of about 0.125% to about 2.5% of the total weight of the liquid composition.

11. The method of claim 11, wherein the composition comprises a carbohydrate, for example in a ratio of trehalose to carbohydrate in the liquid between 4:1 to 0.5:1.

12. The method of claim 1 wherein the drying is carried out at two or more temperatures.

13. The method of claim 1 wherein the mixture is dried to a moisture content of about 1% to about 10%.

14. The method of claim 1 wherein the composition is a vaccine, a part of a vaccine or a precursor of a vaccine.

15. The method of claim 1 wherein the composition is essentially free from non-biobased polymeric excipients and surfactants, for example PMAL-C16.

16. A composition comprising,
a virus;
trehalose;
less than 10% water,
a buffer; and,
optionally, carbohydrate and/or albumin.

17. The composition of claim 16 wherein the virus is a live-attenuated virus and/or a viral vector derived from a vesicular stomatitis virus (VSV) or from an adenovirus (AdV).

18. The composition of claim 16, wherein the carbohydrate is dextran, CMC or pullulan.

19. The composition of claim 16 comprising about 23 to about 300 parts of the trehalose, about 1 to about 10 parts of the buffer, about 12 to about 150 parts of the pulllulan and about 2 to about 35 parts of the albumin, and about 0.0001 to about 0.01 parts of the virus, by weight.

20. The composition of claim 16 that is free from non-biobased polymeric excipients and surfactants, for example PMAL-C16.
